SEQUENCE LISTING (79 sequences)

SEQ ID NO: 1 - skipped

SEQ ID NO: 2 - protein, 14 aa, synthetic construct
  VARIANT 10: note = Any canonical or non-canonical amino acid
APRWDAPLRXPALR

SEQ ID NO: 3-10 - skipped (8 entries)

SEQ ID NO: 11 - protein, 8 aa, synthetic construct
  VARIANT 3: note = Any canonical or non-canonical amino acid
  VARIANT 4: note = Any canonical or non-canonical amino acid
  VARIANT 6: note = Any canonical or non-canonical amino acid
  SITE 1..5: note = Cyclized via Intrachain Disulfide bond
CRXXCXRQ

SEQ ID NO: 12 - protein, 12 aa, synthetic construct
  VARIANT 1: note = R or H
  VARIANT 3: note = Any canonical or non-canonical amino acid
  VARIANT 4: note = Any canonical or non-canonical amino acid
  VARIANT 7: note = Any canonical or non-canonical amino acid
  VARIANT 8: note = Any canonical or non-canonical amino acid
  VARIANT 10: note = Any canonical or non-canonical amino acid
  SITE 5..9: note = Cyclized via Intrachain Disulfide bond
XNXXCRXXCXRQ

SEQ ID NO: 13 - protein, 14 aa, synthetic construct
  VARIANT 1: note = R or H
  VARIANT 3: note = Any canonical or non-canonical amino acid
  VARIANT 4: note = Any canonical or non-canonical amino acid
  VARIANT 7: note = Any canonical or non-canonical amino acid
  VARIANT 8: note = Any canonical or non-canonical amino acid
  VARIANT 10: note = Any canonical or non-canonical amino acid
  VARIANT 13: note = L, L-3-Methyl-Valine or L-4-Methyl-Leucine
  VARIANT 14: note = L, L-3-Methyl-Valine or L-4-Methyl-Leucine
  SITE 5..9: note = Cyclized via Intrachain Disulfide bond
XNXXCRXXCXRQXX

SEQ ID NO: 14 - protein, 17 aa, synthetic construct
  VARIANT 15: note = Any canonical or non-canonical amino acid
  VARIANT 1..8: note = Any canonical or non-canonical amino acid
  VARIANT 1..8: note = This region may encompass 1-8 residues
XXXXXXXXRDPALRXLL

SEQ ID NO: 15 - skipped

SEQ ID NO: 16 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 17 - protein, 24 aa, Homo sapiens
TKCLRREAPRWDAPLRDPALRQLL

SEQ ID NO: 18 - protein, 6 aa, Homo sapiens
APRWDA

SEQ ID NO: 19 - protein, 9 aa, synthetic construct
  VARIANT 2: note = D, F or Any canonical or non-canonical amino acid
  VARIANT 7: note = Q or Any canonical or non-canonical amino acid
  VARIANT 8: note = L or Any canonical or non-canonical amino acid
  VARIANT 9: note = L or Any canonical or non-canonical amino acid
RXPALRXXX

SEQ ID NO: 20 - protein, 8 aa, Homo sapiens
APRWDAPL

SEQ ID NO: 21 - protein, 9 aa, synthetic construct
RDPALRQLL

SEQ ID NO: 22 - protein, 9 aa, synthetic construct
  SITE 7: note = L-Homoleucine
RDPALRXLL

SEQ ID NO: 23 - protein, 17 aa, synthetic construct
  SITE 15: note = L-Homoleucine
APRWDAPLRDPALRXLL

SEQ ID NO: 24 - protein, 5 aa, synthetic construct
  VARIANT 4: note = D or F
PLRXP

SEQ ID NO: 25 - protein, 6 aa, synthetic construct
ALRQLL

SEQ ID NO: 26 - protein, 7 aa, synthetic construct
  VARIANT 3: note = Any canonical or non-canonical amino acid
  VARIANT 7: note = Any canonical or non-canonical amino acid
YKXLRRX

SEQ ID NO: 27 - protein, 7 aa, synthetic construct
YKSLRRK

SEQ ID NO: 28 - protein, 6 aa, synthetic construct
YKSLRR

SEQ ID NO: 29 - protein, 17 aa, Homo sapiens
APRWDAPLRDPALRQLL

SEQ ID NO: 30 - protein, 17 aa, synthetic construct
  SITE 16: note = L-3-Methyl-Valine
  SITE 17: note = 3-cyclobutyl-alanine
APRWDAPLRDPALRQXX

SEQ ID NO: 31 - protein, 15 aa, synthetic construct
APRWDAPLRDPALRQ

SEQ ID NO: 32 - protein, 17 aa, synthetic construct
  SITE 1: note = Residue attached to Monomethyl auristatin E linker attached via a Valine-Citruline p-aminobenzylcarbamate linker
APRWDAPLRDPALRQLL

SEQ ID NO: 33 - protein, 17 aa, synthetic construct
  SITE 1: note = Residue attached to Monomethyl auristatin E linker attached via a Valine-Citruline p-aminobenzylcarbamate linker
DDPRAPWPALQRLALRL

SEQ ID NO: 34 - protein, 17 aa, synthetic construct
DDPRAPWPALQRLALRL

SEQ ID NO: 35 - protein, 6 aa, synthetic construct
HHHHHH

SEQ ID NO: 36 - protein, 17 aa, synthetic construct
QRLARLPRDLLAWPPAD

SEQ ID NO: 37 - protein, 17 aa, synthetic construct
  SITE 1: note = Acetylated residue
APRWDAPLRDPALRQLL

SEQ ID NO: 38 - protein, 17 aa, synthetic construct
  SITE 1: note = Acetylated residue
DDPRAPWPALQRLALRL

SEQ ID NO: 39 - protein, 17 aa, synthetic construct
  SITE 16: note = L-3-Cyclopropyl-Alanine
  SITE 17: note = L-4-Methyl-Leucine
APRWDAPLRDPALRQXX

SEQ ID NO: 40 - protein, 17 aa, synthetic construct
  SITE 16: note = 3-cyclobutyl-alanine
  SITE 17: note = L-4-Methyl-Leucine
APRWDAPLRDPALRQXX

SEQ ID NO: 41 - protein, 17 aa, synthetic construct
  SITE 16: note = L-4-Methyl-Leucine
  SITE 17: note = L-4-Methyl-Leucine
APRWDAPLRDPALRQXX

SEQ ID NO: 42 - protein, 17 aa, synthetic construct
  SITE 16: note = L-Norleucine
  SITE 17: note = L-4-Methyl-Leucine
APRWDAPLRDPALRQXX

SEQ ID NO: 43 - protein, 17 aa, synthetic construct
  SITE 16: note = L-3-Methyl-Valine
  SITE 17: note = L-4-Methyl-Leucine
APRWDAPLRDPALRQXX

SEQ ID NO: 44 - protein, 17 aa, synthetic construct
  SITE 16: note = L-Homoleucine
  SITE 17: note = 3-cyclobutyl-alanine
APRWDAPLRDPALRQXX

SEQ ID NO: 45 - protein, 17 aa, synthetic construct
  SITE 16: note = L-3-Cyclopropyl-Alanine
  SITE 17: note = 3-cyclobutyl-alanine
APRWDAPLRDPALRQXX

SEQ ID NO: 46 - protein, 17 aa, synthetic construct
  SITE 16: note = L-Homoleucine
  SITE 17: note = L-Homoleucine
APRWDAPLRDPALRQXX

SEQ ID NO: 47 - protein, 17 aa, synthetic construct
  SITE 17: note = L-4-Methyl-Leucine
APRWDAPLRDPALRQFX

SEQ ID NO: 48 - protein, 17 aa, synthetic construct
  SITE 17: note = 3-cyclobutyl-alanine
APRWDAPLRDPALRQFX

SEQ ID NO: 49 - protein, 17 aa, synthetic construct
  SITE 17: note = L-4-Methyl-Leucine
APRWDAPLRDPALRQLX

SEQ ID NO: 50 - protein, 17 aa, synthetic construct
  SITE 15: note = L-homoserine
APRWDAPLRDPALRXLL

SEQ ID NO: 51 - protein, 17 aa, synthetic construct
APRWDAPLRFPALRQLL

SEQ ID NO: 52 - protein, 17 aa, synthetic construct
  SITE 16: note = L-4-Methyl-Leucine
APRWDAPLRDPALRQXL

SEQ ID NO: 53 - protein, 17 aa, synthetic construct
  SITE 16: note = L-Homoleucine
  SITE 17: note = L-4-Methyl-Leucine
APRWDAPLRDPALRQXX

SEQ ID NO: 54 - protein, 17 aa, synthetic construct
  SITE 1: note = Acetylated residue
  SITE 16: note = L-3-Methyl-Valine
  SITE 17: note = L-4-Methyl-Leucine
APRWDAPLRDPALRQXX

SEQ ID NO: 55 - protein, 15 aa, synthetic construct
  SITE 1: note = Acetylated residue
  SITE 2: note = L-Norleucine
  SITE 14: note = L-3-Methyl-Valine
  SITE 15: note = L-4-Methyl-Leucine
  SITE 3..11: note = Cyclized via Intrachain Disulfide bond
RXCGPIRSPACRQXX

SEQ ID NO: 56 - protein, 15 aa, synthetic construct
  SITE 1: note = Acetylated residue
  SITE 14: note = L-3-Methyl-Valine
  SITE 15: note = L-4-Methyl-Leucine
  SITE 3..11: note = Cyclized via Intrachain Disulfide bond
RGCIPRRQDACRQXX

SEQ ID NO: 57 - protein, 15 aa, synthetic construct
  SITE 1: note = Acetylated residue
  SITE 8: note = L-Norleucine
  SITE 14: note = L-3-Methyl-Valine
  SITE 15: note = L-4-Methyl-Leucine
  SITE 3..11: note = Cyclized via Intrachain Disulfide bond
RDCVSLRXHVCRQXX

SEQ ID NO: 58 - protein, 15 aa, synthetic construct
  SITE 1: note = Acetylated residue
  SITE 14: note = L-3-Methyl-Valine
  SITE 15: note = L-4-Methyl-Leucine
  SITE 3..11: note = Cyclized via Intrachain Disulfide bond
RDCPSLRAKVCRQXX

SEQ ID NO: 59 - protein, 15 aa, synthetic construct
  SITE 1: note = Acetylated residue
  SITE 8: note = L-Norleucine
  SITE 14: note = L-3-Methyl-Valine
  SITE 15: note = L-4-Methyl-Leucine
  SITE 3..11: note = Cyclized via Intrachain Disulfide bond
RSCTTHRXATCRQXX

SEQ ID NO: 60 - protein, 14 aa, synthetic construct
  SITE 1: note = Acetylated residue
  SITE 10: note = L-Norleucine
  SITE 13: note = L-3-Methyl-Valine
  SITE 14: note = L-4-Methyl-Leucine
  SITE 5..9: note = Cyclized via Intrachain Disulfide bond
RNFHCRAQCXRQXX

SEQ ID NO: 61 - protein, 15 aa, synthetic construct
  SITE 1: note = Acetylated residue
  SITE 14: note = L-3-Methyl-Valine
  SITE 15: note = L-4-Methyl-Leucine
  SITE 3..11: note = Cyclized via Intrachain Disulfide bond
RDCASLRAPPCRQXX

SEQ ID NO: 62 - protein, 15 aa, synthetic construct
  SITE 1: note = Acetylated residue
  SITE 8: note = L-Norleucine
  SITE 14: note = L-3-Methyl-Valine
  SITE 15: note = L-4-Methyl-Leucine
  SITE 3..11: note = Cyclized via Intrachain Disulfide bond
RVCANHRXNTCRQXX

SEQ ID NO: 63 - protein, 15 aa, synthetic construct
  SITE 1: note = Acetylated residue
  SITE 14: note = L-3-Methyl-Valine
  SITE 15: note = L-4-Methyl-Leucine
  SITE 3..11: note = Cyclized via Intrachain Disulfide bond
EWCGLHNHPTCRQXX

SEQ ID NO: 64 - protein, 15 aa, synthetic construct
  SITE 1: note = Acetylated residue
  SITE 8: note = L-Norleucine
  SITE 14: note = L-3-Methyl-Valine
  SITE 15: note = L-4-Methyl-Leucine
  SITE 3..11: note = Cyclized via Intrachain Disulfide bond
RWCENRRXFTCRQXX

SEQ ID NO: 65 - protein, 15 aa, synthetic construct
  SITE 1: note = Acetylated residue
  SITE 14: note = L-3-Methyl-Valine
  SITE 15: note = L-4-Methyl-Leucine
  SITE 3..11: note = Cyclized via Intrachain Disulfide bond
RTCNHLRNPACRQXX

SEQ ID NO: 66 - protein, 15 aa, synthetic construct
  SITE 1: note = Acetylated residue
  SITE 8: note = L-Norleucine
  SITE 14: note = L-3-Methyl-Valine
  SITE 15: note = L-4-Methyl-Leucine
  SITE 3..11: note = Cyclized via Intrachain Disulfide bond
RWCENQRXTVCRQXX

SEQ ID NO: 67 - protein, 14 aa, synthetic construct
  SITE 1: note = Acetylated residue
  SITE 13: note = L-3-Methyl-Valine
  SITE 14: note = L-4-Methyl-Leucine
  SITE 5..9: note = Cyclized via Intrachain Disulfide bond
HNFYCRKHCQRQXX

SEQ ID NO: 68 - protein, 14 aa, synthetic construct
  SITE 1: note = Acetylated residue
  SITE 8: note = L-Norleucine
  SITE 13: note = L-3-Methyl-Valine
  SITE 14: note = L-4-Methyl-Leucine
  SITE 5..9: note = Cyclized via Intrachain Disulfide bond
RNIFCRKXCERQXX

SEQ ID NO: 69 - protein, 14 aa, synthetic construct
  SITE 1: note = Acetylated residue
  SITE 4: note = L-Norleucine
  SITE 13: note = L-3-Methyl-Valine
  SITE 14: note = L-4-Methyl-Leucine
  SITE 3..10: note = Cyclized via Intrachain Disulfide bond
AQCXGRNRACRQXX

SEQ ID NO: 70 - protein, 15 aa, synthetic construct
  SITE 1: note = Acetylated D-Pro
  SITE 3: note = D-Glu
  SITE 4: note = D-Lys
  SITE 5: note = D-Leu
  SITE 6: note = D-Gln
  SITE 8: note = D-Val
  SITE 9: note = D-Arg
  SITE 12: note = D-Arg
  SITE 14: note = L-3-Methyl-Valine
  SITE 15: note = L-4-Methyl-Leucine
  SITE 2..11: note = Cyclized via Intrachain Disulfide bond
PCEKLQRVRTCREXX

SEQ ID NO: 71 - protein, 9 aa, synthetic construct
  SITE 1: note = Acetylated residue
  SITE 7: note = L-Homoleucine
RDPALRXLL

SEQ ID NO: 72 - protein, 14 aa, synthetic construct
  VARIANT 1: note = R, H or Any canonical or non-canonical amino acid
  VARIANT 3: note = Any canonical or non-canonical amino acid
  VARIANT 4: note = Any canonical or non-canonical amino acid
  VARIANT 7: note = Any canonical or non-canonical amino acid
  VARIANT 8: note = Any canonical or non-canonical amino acid
  VARIANT 10: note = Any canonical or non-canonical amino acid
  VARIANT 13: note = L or Any canonical or non-canonical amino acid
  VARIANT 14: note = L or Any canonical or non-canonical amino acid
XNXXCRXXCXRQXX

SEQ ID NO: 73 - protein, 17 aa, synthetic construct
  VARIANT 10: note = D, F or Any canonical or non-canonical amino acid
  VARIANT 15: note = Q or Any canonical or non-canonical amino acid
  VARIANT 16: note = L or Any canonical or non-canonical amino acid
  VARIANT 17: note = L or Any canonical or non-canonical amino acid
APRWDAPLRXPALRXXX

SEQ ID NO: 74 - protein, 17 aa, synthetic construct
  VARIANT 1: note = Any canonical or non-canonical amino acid
  VARIANT 3: note = R or Any canonical or non-canonical amino acid
  VARIANT 4: note = W or Any canonical or non-canonical amino acid
  VARIANT 5: note = D or Any canonical or non-canonical amino acid
  VARIANT 9: note = R or Any canonical or non-canonical amino acid
  VARIANT 10: note = D, F or Any canonical or non-canonical amino acid
  VARIANT 14: note = R or Any canonical or non-canonical amino acid
  VARIANT 15: note = Q or Any canonical or non-canonical amino acid
  VARIANT 16: note = L or Any canonical or non-canonical amino acid
  VARIANT 17: note = L or Any canonical or non-canonical amino acid
XPXXXAPLXXPALXXXX

SEQ ID NO: 75 - protein, 13 aa, synthetic construct
QLYENKPRRPYIL

SEQ ID NO: 76 - protein, 17 aa, synthetic construct
  SITE 16: note = 3-cyclobutyl-alanine
  SITE 17: note = L-4-Methyl-Leucine
APRWDAPLRDPALRQXX

SEQ ID NO: 77 - protein, 22 aa, synthetic construct
  SITE 1: note = Beta-Alanine attached to Valine-citruline p-aminobenzylcarbamate linker via a Monomethyl auristatin E linker
  SITE 2: note = Sarcosine
  SITE 3: note = Sarcosine
  SITE 4: note = Sarcosine
  SITE 5: note = Sarcosine
XXXXXAPRWDAPLRDPALRQLL

SEQ ID NO: 78 - protein, 22 aa, synthetic construct
  SITE 1: note = Beta-Alanine attached to Valine-citruline p-aminobenzylcarbamate linker via a Monomethyl auristatin E linker
  SITE 2: note = Sarcosine
  SITE 3: note = Sarcosine
  SITE 4: note = Sarcosine
  SITE 5: note = Sarcosine
  SITE 21: note = L-3-Methyl-Valine
  SITE 22: note = 3-cyclobutyl-alanine
XXXXXAPRWDAPLRDPALRQXX

SEQ ID NO: 79 - protein, 15 aa, synthetic construct
  SITE 1: note = Acetylated residue
  SITE 6: note = Sarcosine x4 Beta-Ala Valine-citruline p-aminobenzylcarbamate linker Monomethyl auristatin E linker attached to side chain of Lysine
  SITE 8: note = L-Norleucine
  SITE 14: note = L-3-Methyl-Valine
  SITE 15: note = L-4-Methyl-Leucine
  SITE 3..11: note = Cyclized via Intrachain Disulfide bond
RWCENKRXFTCRQXX

(19) United States
(12) Patent Application Publication
(10) Pub. No.: US 2024/0285785 A1
(43) Pub. Date: Aug. 29, 2024

(54) TARGETING SORTILIN

(71) Applicant: ProteinQure Inc., Toronto (CA)

(72) Inventors: Robert Aron Broom, Ottawa (CA); Glenn Larry Butterfoss, Glen Mills, PA (US); Christopher Edward Ing, Toronto (CA); Tian Yu Lu, Toronto (CA); Francine Evelyn Lui, Toronto (CA); Serban Popa, Toronto (CA); Tracy Anne Stone, Toronto (CA); Ozge Yoluk, Toronto (CA); David William White, Walpole, MA (US); Andrew Zhai, Toronto (CA); Sungwon Hwang, North York (CA)

(21) Appl. No.: 18/531,211

(22) Filed: Dec. 6, 2023

Related U.S. Application Data
(60) Provisional application No. 63/430,643, filed on Dec. 6, 2022.

(51) Int. Cl. A61K 47/64; A61K 45/06; A61K 51/08

(52) U.S. Cl. CPC A61K 47/64; A61K 45/06; A61K 51/08

(57) ABSTRACT

Disclosed herein are sortilin binding agents, including conjugate agents comprising a sortilin binding moiety, directly or indirectly conjugated with a payload moiety, compositions comprising the same as well as methods of making and using the same.

### CROSS-REFERENCE TO RELATED APPLICATIONS

The present application claims the benefit of U.S. Provisional Application No. 63/430,643 filed Dec. 6, 2022, the contents of which are hereby incorporated by reference in their entirety.

### SEQUENCE LISTING

The present specification makes reference to a Sequence Listing (submitted electronically as a .xml file named “2017417-0005_ST26.xml” on Apr. 19, 2024). The .xml file was generated on Mar. 7, 2024 and is 135,921 bytes in size. The entire contents of the Sequence Listing are herein incorporated by reference.

### BACKGROUND

Sortilin, also known as neurotensin receptor-3 (NTSR3), has transmembrane and soluble forms and has been reported to play a role in a variety of physiological processes and conditions, including certain cancers, cardiovascular disease, Alzheimer's disease, diabetes, depression, etc.

### SUMMARY

The present disclosure provides technologies for targeting sortilin, for example to deliver a payload to cells that express it (e.g., have sortilin on their surface). Among other things, the present disclosure provides sortilin-targeting agents, and conjugates thereof (e.g., wherein a payload is conjugated thereto).

In some embodiments, provided sortilin binding agents (and/or conjugates thereof) are demonstrated to bind to sortilin with high affinity and/or to have useful stability characteristics, including for example in serum.

In some embodiments, provided sortilin binding agents (and/or conjugates thereof) are demonstrated to effectively deliver a payload (e.g., a therapeutic payload, which may in some embodiments be a toxic payload) to cells (e.g., to sortilin-expressing cells); in some embodiments, a correlation between affinity for sortilin and effective cell killing (e.g., by delivery of a toxic payload) is demonstrated.

In some embodiments, provided sortilin binding agents (and/or conjugates thereof) are demonstrated to effectively deliver a payload (e.g., a toxic payload) to cells within a range of doses between a minimally active dose and a maximum tolerated dose; in some embodiments, such range is demonstrated to span at least 3 fold in mice.

In some embodiments, provided sortilin binding agents (and/or conjugates thereof) are characterized by one or more of:
(i) affinity (Kd) for human sortilin up to 1 μM when assessed by fluorescence polarization;
(ii) in a competitive binding assay, IC₅₀ is less than about 12 μM
(iii) when maintained under murine serum, displays stability >3.16 minutes.

Among other things, the present disclosure provides an insight relating to the source of a problem in prior efforts to develop sortilin binding agents, and in particular prior attempts to develop conjugates that bind to sortilin on cells expressing it and deliver a payload into such cells. For example, the present disclosure observes that certain prior efforts to target sortilin have focused on developing agents structurally related to sortilin ligand(s) other than progranulin (e.g., to certain bacterial cell penetrant proteins and/or neurotensin). Moreover, even those efforts that have focused on developing agents structurally related to progranulin have gone down a different path.

For example, in certain embodiments, the present disclosure provides and/or utilizes sortilin-binding peptides having an amino acid sequence that corresponds substantially to a fragment of human progranulin but includes substitution(s) of one or more amino acids at positions P3, P4, P10 and/or P17 as described herein. Alternatively or additionally, in certain embodiments, the present disclosure provides and/or utilizes sortilin-binding peptides having an amino acid sequence that corresponds substantially to a fragment of human progranulin but includes one or more substitutions with non-natural amino acids (in some embodiments specifically at one or more positions corresponding to one or more of P15, P16, and P17, including specifically P16 and/or P17—e.g., at one or more of the last 3 C-terminal residues). Still further alternatively or additionally, in certain embodiments, the present disclosure provides and/or utilizes cyclic sortilin-binding peptides, including cyclic peptides having certain amino acid sequence feature(s) found in human progranulin, such as specifically certain C-terminal sequences.

The present disclosure appreciates that certain other approaches to development of sortilin binding agents have suggested that residues corresponding to P1-P6 as found in human progranulin should be deleted; the present disclosure, in some embodiments, takes a different approach, including these residues. That is, the C-terminal twenty four amino acids of wild type human progranulin (SEQ ID NO: 17) are:

| P-6 | P-5 | P-4 | P-3 | P-2 | P-1 | P0 | P1 | P2 | P3 | P4 | P5 | P6 | P7 | P8 | P9 | P10 | P11 | P12 | P13 | P14 | P15 | P16 | P17 |
|---|---|---|---|---|---|---|---|---|---|---|---|---|---|---|---|---|---|---|---|---|---|---|---|
| T | K | C | L | R | R | E | A | P | R | W | D | A | P | L | R | D | P | A | L | R | Q | L | L |

Certain prior efforts to develop sortilin-binding peptides based on progranulin (in particular, for example, as described in one or more of WO2017/088058; WO2018/213928; and/or WO2020/037434) fail to appreciate that residues P1-P6 (APRWDA (SEQ ID NO: 18)) can beneficially contribute to sortilin binding, and even recommend potential removal or substitution of certain of these residues. Such teachings lead away from development of peptides such as certain provided peptides, which include such residues (e.g., an APRWDA element (SEQ ID NO: 18)).

Among other things, the present disclosure provides an insight that the C-terminal part of certain provided peptides, e.g., the sequence, R(D/F/X)PALR(Q/X)(L/X)(L/X) (SEQ ID NO: 19), may be responsible for sortilin binding, and, for example, contains the major driver for binding affinity, and, furthermore, that the N-terminal part of the sequence, APRWDAPL (SEQ ID NO: 20), may bind to a secondary site in the target, contributing to the binding affinity but to a lesser extent. Thus, the present disclosure identifies the source of a problem with certain strategies as proposed by others, including for example with strategies that may propose peptides of different length and/or sequence, and/or peptides that may not include part or all of the APRWDAPL sequence (SEQ ID NO: 20).

In some embodiments, a peptide according to current disclosure has XⁿRDPALRXLL sequence (SEQ ID NO: 14), wherein X is any canonical or non-canonical amino acid according to current disclosure, and n is 0, 1, 2, 3, 4, 5, 6, 7 or 8. In some embodiments, for example, a peptide according to current disclosure has an amino acid sequence that is or comprises RDPALRQLL sequence (SEQ ID NO: 21). In some embodiments, for example, a peptide according to current disclosure has an amino acid sequence that is or comprises RDPALR(B43)LL (SEQ ID NO: 22).

As noted above, among other things, the present disclosure demonstrates that, in some embodiments, including an APRWDAPL sequence (SEQ ID NO: 20) can improve one or more aspects or features (e.g., in some embodiments, affinity, on-rate and/or off rate, etc) of sortilin binding by a provided peptide (e.g., a provided sortilin binding moiety). In some particular embodiments, for example, the present disclosure demonstrates that including APRWDAPL (SEQ ID NO: 20) can improve sortilin-affinity of an RDPALR(B43)LL peptide (SEQ ID NO: 22). That is, in particular, the present disclosure documents that a peptide having an amino acid sequence compared to APRWDAPLRDPALR(B43)LL (SEQ ID NO: 23) can show improved sortilin affinity as compared with a peptide having the amino acid sequence RDPALR(B43)LL (SEQ ID NO: 22).

In some embodiments, the present disclosure provides peptides whose amino acid sequence includes an APRWDA element (SEQ ID NO: 18). In some such embodiments, such peptides are linear peptides; alternatively or additionally, in some embodiments, such peptides include one or more of (i) an PLR(D/F)P element (SEQ ID NO: 24); (ii) an ALRQLL element (SEQ ID NO: 25), or a variant of ALRQLL (SEQ ID NO: 25) where one or more of Q or one or both of the Ls is substituted with a non-natural amino acid.

Furthermore, certain prior efforts to develop sortilin-binding peptides based on progranulin (in particular, for example, as described in one or more of WO2017/088058; WO2018/213928; and/or WO2020/037434) recommend including amino acids corresponding to residues at positions P-6 to P0 of human sortilin, and specifically recommend a YKXLRRX (SEQ ID NO: 26) (e.g., YKSLRRK (SEQ ID NO: 27)) element be included. The present disclosure provides alternative approaches to sortilin-binding peptides. In some embodiments, such approaches include peptides that do not include residues corresponding to amino acids more than 17 residues N-terminal of the human progranulin C-terminus and/or that lack a YKXLRRX (SEQ ID NO: 26) (e.g., YKSLRRK (SEQ ID NO: 27)) sequence element; in some embodiments, provided peptides lack a YKX (e.g., YKS), KXL (e.g., KSL), XLR (e.g., SLR), LRR, and/or LRX (e.g. LRK) element.

Still further, it is worth noting that WO2020/037434, which describes itself as providing peptides that target sortilin (and references prior publications WO2017/088058 and WO2018/213928), identifies three specific types of such peptides: (1) those derived from a bacterial cell penetrant protein, (2) those based on an optimized primary sequence derived from progranulin; and (3) those based on an optimized primary sequence derived from neurotensin. As already noted above, the progranulin sequences identified by WO2020/037434 as the basis for desirable sortilin-binding peptides differ from those utilized herein; moreover, peptides and/or conjugates described herein have different structures than those taught or suggested in WO2020/037434.

Particularly in this context, contributions of the present disclosure surprisingly demonstrate particular utility of sortilin-binding peptides as described herein, including, for example peptides (1) having an amino acid sequence that (a) includes residues corresponding to sequences found in a C-terminal fragment of human progranulin; (b) does not include residues corresponding to amino acids more than 17 residues N-terminal of the human progranulin C-terminus; (c) does not include a sequence element that comprises or consists of YKSLRR (SEQ ID NO: 28); (d) does not include a sequence element that comprises or consists of YKS, KSL, SLR, or LRR; (e) includes an ALRQLL element (SEQ ID NO: 25); (f) includes a variant of an ALRQLL sequence element (SEQ ID NO: 25), wherein one or more of (i) Q; or (ii) one or both of the Ls is substituted with a non-natural amino acid; (2) that are cyclic; (3) that are fewer than 20 amino acids long, such as that are about 17 amino acids long, or are exactly 17 amino acids long; and/or that otherwise have structural and/or functional characteristics as described herein.

Among other things, in some particular embodiments, the present disclosure provides conjugates comprising:
(a) polypeptide; and
(b) a payload; and
(c) optionally, a linker,
   wherein the polypeptide includes a sortilin binding moiety that:
(a) has a length within a range of about 12 to about 20 amino acids and includes a characteristic sequence represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein each X is independently any canonical or non-canonical amino acid.

In some embodiments, provided sortilin binding moiety corresponds to a C-terminal fragment of progranulin, or a variant thereof, and includes not more than about 20 contiguous residues corresponding to contiguous progranulin residues.

In some embodiments, a provided sortilin binding moiety is or comprises a cyclic peptide. In some embodiments, a provided sortilin binding moiety includes a characteristic sequence which is represented by:

(SEQ ID NO: 3)
(R/N) X²⁻³ C X⁰⁻¹ R (Q/E).

In some embodiments, a provided sortilin binding moiety is or comprises a linear peptide. In some embodiments, a provided sortilin binding moiety is or comprises a linear peptide. In some embodiments, a provided sortilin binding moiety includes a characteristic sequence represented by SEQ ID NO:2. In some embodiments, a provided sortilin binding moiety includes a characteristic sequence represented by:

(SEQ ID NO: 4)
(R/N) X²⁻³ L X⁰⁻¹ R (Q/B43/B50).

In some embodiments, a provided sortilin binding agent (and/or conjugate thereof) or a sortilin binding moiety is or comprises an amino acid sequence APRWDAPLRDPALRQLL (SEQ ID NO: 29). In some embodiments, a provided sortilin binding agent (and/or conjugate thereof) or a sortilin binding moiety is or comprises an amino acid sequence APRWDAPLRDPALRQ(B13)(G48) (SEQ ID NO: 30).

In some embodiments, a provided nucleic acid comprises a nucleotide sequence that includes a coding region that encodes a peptide comprising a sequence of APRWDAPLRDPALRQLL (SEQ ID NO: 29). In some embodiments, a provided nucleic acid comprises a nucleotide sequence that includes a coding region that encodes a peptide comprising a sequence of APRWDAPLRDPALRQ (SEQ ID NO: 31).

In some embodiments, a provided linker is a VCPAB linker.

In some embodiments, a provided payload is MMAE.

These, and other aspects encompassed by the present disclosure, are described in more detail below and in the claims.

### BRIEF DESCRIPTION OF THE DRAWING

FIG. 1 provides the amino acid sequence of human progranulin (SEQ ID NO:16).

FIG. 2 illustrates an exemplary conjugate agent provided in accordance with the present disclosure.

FIG. 3 demonstrates binding affinity of PRGN_based homing peptides determined by (a) direct titration and (b) competition binding in an FP binding assay.

FIG. 4 demonstrates Binding affinity of PRGN_WT (sometimes referred to as PRGN_C18 or PRGN_WT_C18) determined by Grating-Coupled Interferometry. (a) Protein-loaded format; and (b) Peptide-loaded format.

FIG. 5 demonstrates Binding affinity of PRGN_RL determined by Grating-Coupled Interferometry. (a) Protein-loaded format; and (b) Peptide-loaded format.

FIG. 6 demonstrates Biophysical characterization of homing peptides by (a) dynamic light scattering, wherein homing peptides do not exhibit aggregation; and (b) circular dichroism, wherein homing peptides are unstructured, random coil in buffered solution.

FIG. 7 demonstrates biophysical characterization of peptide drug conjugates (PDCs) by (a) dynamic light scattering, wherein PDCs do not exhibit aggregation; and (b) circular dichroism, wherein PDCs are unstructured, random coil in buffered solution.

FIG. 8 demonstrates binding of PDCs to SORT1 in a FP binding assay.

FIG. 9 illustrates Chemical structure of PRGN_PDC_WT (SEQ ID NO: 32) and PRGN_PDC_RL (SEQ ID NO: 33).

FIGS. 10a-c demonstrate characterization of PRGN_WT and PRGN_PDC_WT by LC-MS. Readout is devoid of contaminants and fragments correspond to mass of parent compound. FIG. 10a is an LC-MS trace for PRGN_WT; FIG. 10b is an MS/MS spectra for PRGN_PDC_WT; and FIG. 10c is an HPLC chromatograms for PRGN_PDC_WT.

FIG. 11 demonstrates potency of PGRN-based PDCs in 72 h cell killing assay with a) HCC70; and b) MDA-MB-231 cells.

FIG. 12 demonstrates characterization of PRGN_based PDCs in an in vivo maximum tolerated dose (MTD) n=3. (a) WT_PDC; (b) RL_PDC.

FIG. 13 demonstrates PQ designed peptides binding with SORT1 identified through peptide microarray. (a) Linear peptides. His-tag positive controls were printed at positions A1-A5.; and (b) Cyclic peptides. His-tag positive controls were printed at positions A7-A11, T1-T2 and T30.

FIG. 14 demonstrates biophysical characterization of linear PQ designed peptides by (a) dynamic light scattering where aggregating peptides (colored bars) were removed from workflow; and (b) representative circular dichroism traces.

FIG. 15 demonstrates Biophysical characterization of cyclic PQ designed peptides by (a) dynamic light scattering and (b) representative circular dichroism traces.

FIG. 16 demonstrates affinity of PQ designed linear peptides to SORT1 in a fluorescence polarization assay. PRGN_WT and natural ligand neurotensin are highlighted in red and blue, respectively. Computationally designed linear peptides exhibit improved affinity to SORT1 relative to control PRGN_WT.

FIG. 17 demonstrates affinity of PQ designed cyclic peptides to SORT1 in a fluorescence polarization assay.

FIG. 18 illustrates affinities and murine serum stabilities of the full list of SORT1 binding peptides. PRGN_WT and natural ligand neurotensin are highlighted in green and red, respectively.

FIG. 19 illustrates Sequence logo of cyclic peptide. (a) Cyclic group 1. Following amino acids are shown as their natural variants: F02, B13, and Nle as L,L and M, respectively. And (b) cyclic group 2. Following amino acids are shown as their natural variants: F02, B13, and Nle as L,L and M respectively.

FIG. 20 illustrates internalization and lysosomal colocalization of PRGN_WT homing peptide conjugated to fluorescent label AF488 in MDA-MB-231 TNBC cells. Two different representative fields are shown of MDA-MB-231 cells treated with Lysotracker (red) AF488-PRGN_WT (green) and Hoechst nuclear stain (blue). The merged channel is depicted on the right with yellow pixels demonstrating co-localization of AF488-PRGN_WT with lysosomes.

FIG. 21 illustrates the in-vivo efficacy of PRGN_WT_PDC as well as free Monomethyl auristatin E (MMAE) in a MDA-MB-231 murine xenograft model, n=6 for each treatment arm. (a) Depicts tumor growth curves following treatment with three different doses of PRGN_WT_PDC as well as free MMAE dosed at equivalency to 3 mg/kg WT_PDC. (b) Depicts body weight changes as a result of treatment with PRGN_WT_PDC and free MMAE. (P<0.0001; Two Way ANOVA with Sidek's multiple comparison test).

FIG. 22 illustrates the in-vivo efficacy of PRGN_WT_PDC, Arb-SAR-Q15-PDC, Arb_cyclic_G23-PDC, a control PRGN_scr_PDC, and free MMAE in a MDA-MB-231 murine xenograft model, n=8 for each treatment arm. (a) Depicts tumor growth curves following treatment with PRGN_WT_PDC, Arb-SAR-Q15-PDC, Arb-cyclic-G23-PDC, a control PRGN_scr_PDC, and free MMAE dosed at molar equivalency to 3 mg/kg of PDC. (b) Depicts body weight changes as a result of treatment group. (**P≤0.01; Two Way ANOVA with Sidek's multiple comparison test).

FIG. 23 demonstrates characterization of peptide drug conjugates PRGN_WT_PDC, Arb-SAR-Q15-PDC, and Arb_cyclic_G23-PDC, along with vehicle and free MMAE in an in-vivo hematotoxicity assay n=3. (*P≤0.05, **P≤0.01, ***P≤0.001, ****P≤0.0001).

### DEFINITIONS

In order to further define this invention, the following terms and definitions are herein provided.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” refers to the administration of a composition (e.g., a compound [e.g., a conjugate] as described herein or a preparation that includes or otherwise delivers such compound) to a subject or system, or to a cell or tissue thereof. Administration to an animal subject (e.g., to a human) can be by an appropriate route, such as one described herein. In some embodiments, administration may be local. In some embodiments, administration may be systemic. In some embodiments, administration may be enteral. In many embodiments, administration may be parenteral. In some particular embodiments, parenteral administration may be intradermal, intramuscular, intrathecal, intravenous, subcutaneous, etc.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference” ]. In some embodiments, affinity may be assessed relative to a contemporaneous reference; in some embodiments, affinity may be assessed relative to a historical reference. Typically, when affinity is assessed relative to a reference, it is assessed under comparable conditions.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” or “canonical amino acid” amino acid refers to any of the twenty standard L-amino acids commonly found in naturally occurring peptides. “Nonstandard amino acid” or “non-canonical amino acid” refers to any amino acid, other than the standard amino acids, regardless of whether it is prepared synthetically or obtained from a natural source. Particular nonstandard amino acids that may be especially useful in accordance with certain embodiments of the present disclosure are provided herein (see, for example, Table 2-7) and, in some embodiments, particular sites at which certain such nonstandard amino acids may be most useful are designated.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include one or more modifications on an Fc domain. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of dog, cat, mouse, rabbit, primate, or human antibodies. In some embodiments, antibody sequence elements are human, humanized, primatized, chimeric, etc, as is known in the art. In some embodiments, the term “antibody” (or “antibody moiety”) as used herein, can refer in appropriate embodiments (unless otherwise stated or clear from context) to any of the art-known or developed constructs or formats for utilizing antibody structural and functional features in alternative presentation. For example, in some embodiments, an “antibody” (or antigen-binding elements thereof) may be utilized in accordance with the present invention in a single-chain format, or another format that is smaller than a full antibody or is otherwise particularly amenable to inclusion in a conjugate as described herein). In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated: Two events or entities are “associated” with one another, as that term is used herein, if the presence, level, degree, type and/or form of one is correlated with that of the other. For example, a particular entity (e.g., polypeptide, etc) is considered to be associated with a particular cell type or a particular disease, disorder, or condition, if its presence, level and/or form correlates with identity of such cell type or with incidence of, susceptibility to, severity of, stage of, etc such disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). Binding between two entities may be considered “specific” if, under the conditions assessed, the relevant entities are more likely to associate with one another than with other available binding partners. In some embodiments, an agent (e.g., a peptide as described herein) that binds specifically with a particular target (e.g., sortilin), is said to “home to” or to be a “homing” agent for, such target.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Cell surface factor. The term “cell surface factor” as used herein, refers to a factor (e.g., that is or comprises a polypeptide) that is present on the surface of cell(s) of interest (e.g., of target cell(s) as described herein which, in many embodiments, may be cells expressing sortilin, such as cancer cells expressing sortilin). In some embodiments, a cell surface factor is preferentially present on the surface of target cell(s) (e.g., cancer cells) as compared with cells of one or more other tissues. In some embodiments, a cell surface factor is present on certain non-target cells in addition to target cells. In some embodiments, a cell surface factor is not preferentially or specifically present on relevant target cells of interest. In some embodiments, a cell surface factor is or comprises a receptor. In some embodiments, a cell surface factor is internalized when bound by one or more particular ligands (e.g., with a sortilin binding moiety as described herein). In some embodiments, a cell surface factor may interact with (e.g., bind to, form a complex with, etc) one or more other components of a cell (e.g., with one or more cell membrane components and/or one or more cell surface components and/or one or more cell-internal components) on whose surface it is found. In some embodiments, a cell surface factor, and/or a particular form or variant thereof, and/or a cell surface factor of any of the foregoing, may be associated with a particular cell state or condition (e.g., stage of development, disease state, etc).

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Conservative: As used herein, the term “conservative” refers to instances describing a conservative amino acid substitution, including a substitution of an amino acid residue by another amino acid residue having a side chain R group with similar structural, chemical (e.g., charge or hydrophobicity), and/or functional properties. In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet et al., Science 256:1443, 1992, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein. In some embodiments, a “conservative” substitution is considered a “homologous” residue for purposes of calculating percent homology between amino acid sequences.

Conjugate agent. The term “conjugate agent” as used herein refers to an agent that has been engineered to link distinct moieties to one another. In accordance with the present disclosure, a conjugate agent typically includes at least one sortilin binding moiety as described herein directly or indirectly conjugated with at least one payload moiety. In some embodiments, a sortilin binding moiety is or comprises a peptide. In some embodiments, a conjugate agent includes at least one sortilin binding moiety, at least a first payload moiety, and at least one other moiety, which other moiety may be for example, a second sortilin binding moiety, a second payload moiety, or another moiety, such as a binding moiety for a target other than sortilin. In some embodiments, a conjugate agent includes at least two binding moieties, at least one of which is a sortilin binding moiety. Alternatively or additionally, in some embodiments, a conjugate agent includes at least two payload moieties (which, in some embodiments, may be two or more of the same payload moiety and in some embodiments may be two or more different payload moieties). In some embodiments, a conjugate agent includes a plurality of sortilin binding moieties; in some such embodiments, all of the plurality are the same sortilin binding moiety, in some such embodiments, the plurality includes at least two different sortilin binding moieties, in some such embodiments, each sortilin binding moiety of the plurality is different from each other sortilin binding moiety of the plurality. In some embodiments, a conjugate agent includes a plurality of payload moieties; in some such embodiments, all of the plurality are the same payload moiety, in some such embodiments, the plurality includes at least two different payload moieties, in some such embodiments, each payload moiety of the plurality is different from each other payload moiety of the plurality. In some particular embodiments, a single sortilin binding moiety may be conjugated to multiple payload moieties. In some such embodiments, all such payload moieties are conjugated at the same site on the sortilin biding moiety. In other such embodiments, at least two such payload moieties are conjugated at different sites on the sortilin binding moiety; in some such embodiments, each payload moiety is conjugated at a different site on the sortilin binding moiety. Alternatively or additionally, with respect to each such embodiment in which a plurality of payload moieties is conjugated to a single sortilin binding moiety, in some embodiments all of the plurality are the same payload moiety, in some such embodiments, the plurality includes at least two different payload moieties, in some such embodiments, each payload moiety of the plurality is different from each other payload moiety of the plurality. In some particular embodiments, a single payload moiety is conjugated to multiple sortilin binding moieties. In some such embodiments, all such sortilin binding moieties are conjugated at the same site on the payload moiety. In other such embodiments, at least two such sortilin binding moieties are conjugated at different sites on the payload moiety; in some such embodiments, each sortilin binding moiety is conjugated at a different site on the payload moiety. Alternatively or additionally, with respect to each such embodiment in which a plurality of sortilin binding moieties is conjugated to a single payload moiety, in some embodiments all of the plurality are the same sortilin binding moiety, in some such embodiments, the plurality includes at least two different sortilin binding moieties, in some such embodiments, each sortilin binding moiety of the plurality is different from each other sortilin binding moiety of the plurality.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a sortilin binding moiety corresponds to a C-terminal fragment of progranulin or a variant thereof and includes not more than about 20 contiguous residues corresponding to contiguous progranulin residues.

Designed: As used herein, the term “designed” refers to an agent (i) whose structure is or was selected by the hand of man; (ii) that is produced by a process requiring the hand of man; and/or (iii) that is distinct from natural substances and other known agents.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments described abd.ir utilized herein, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Excipient: as used herein, refers to a non-therapeutic agent that may be included in a pharmaceutical composition, for example to provide or contribute to a desired consistency or stabilizing effect.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer, for example contiguously connected as they are in the whole. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Homology: As used herein, the term “homology” refers to the overall relatedness between polymeric molecules, e.g., between polypeptide molecules. In some embodiments, polymeric molecules are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar, e.g., are 80%, 85%, 90%, 95%, or 99% identical if substitutions with a “similar” residue are not counted as differences.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The residues at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids. In particular embodiments, a peptide has a length within a range of about 12 to about 20 amino acids. In some embodiments, a peptide has a length of about 15 to about 20 amino acids, or about 15 to about 18 amino acids. In some embodiments, a peptide has a length of about 17 amino acids, such as a length of 17 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc, which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sortilin binding moiety: The term “sortilin binding moiety” as used herein, refers to a moiety that, when contacted with a system that includes sortilin, or a relevant portion thereof, can be determined or concluded to bind specifically with such sortilin. In some embodiments, the system is an in vitro system. In some embodiments, the system may include cells (e.g., cells determined or known to express sortilin). In some embodiments, such cells (which may be considered “target cells of interest” may be in culture, in a tissue, and/or in an organism). In many embodiments, a sortilin binding moiety is characterized (e.g., determined or concluded to bind specifically with sortiling) binds to a cell surface factor (e.g., to a factor that is preferentially or specifically found on surface(s) of cells known to express sortilin. In some embodiments, binding of a sortilin binding moiety to a cell surface factor results in internalization of a sortilin binding moiety. Typically, a sortilin binding moiety useful in accordance with the present disclosure retains its specific binding character when included in a conjugate agent as described herein; in some embodiments, binding of such a conjugate agent to a relevant cell surface factor results in internalization of a conjugate agent. In some embodiments, a sortilin binding moiety binds specifically to a factor on the surface of cancer cells expressing sortilin.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Subject As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial sequence identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Treat: As used herein, the terms “treat,” “treated,” and “treating” refer to delaying onset of and/or reducing severity and/or frequency of one or more undesired physiological events or states (e.g., which may be indicative of a particular condition, disorder, or disease) and/or achieving a particular beneficial or desired physiological or result(s) and/or administration of a regimen or therapy demonstrated or reasonably expected to accomplish such delaying, reducing or achieving. In some embodiments, a beneficial or desired clinical results include, but are not limited to, alleviation of one or more symptoms; diminishment of the extent of a condition, disorder, or disease; stabilization (e.g., not worsening) of a state of a condition, disorder, or disease; delay in onset or slowing of progression of a condition, disorder, or disease; amelioration of the condition, disorder, or disease state, remission (whether partial or total), whether detectable or undetectable; an amelioration of at least one measurable physical parameter, not necessarily discernible by the patient; enhancement or improvement of condition, disorder, or disease, etc. In some embodiments, treatment may involve eliciting a clinically significant response without excessive side effects. In some embodiments, treatment may be or comprise prolonging survival as compared to an expected survival if not receiving treatment.

Variant: The term “variant”, as used herein, refers to a molecule or entity (e.g., that are or comprise a nucleic acid, protein, or small molecule) that shows significant structural identity with a reference molecule or entity but differs structurally from the reference molecule or entity, e.g., in the presence or absence or in the level of one or more chemical moieties as compared to the reference molecule or entity. In some embodiments, a “variant” may be referred to as a “derivative”. In some embodiments, a variant differs functionally from its reference molecule or entity. In many embodiments, whether a particular molecule or entity is properly considered to be a “variant” of a reference is based on its degree of structural identity with the reference molecule. As will be appreciated by those skilled in the art, a biological or chemical reference molecule in typically characterized by certain characteristic structural elements. A variant, by definition, is a distinct molecule or entity that shares one or more such characteristic structural elements but differs in at least one aspect from the reference molecule or entity. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION

Disclosed herein, inter alia, are sortilin binding agents (e.g., peptides) and various compositions that include them. Among other things, the present disclosure provides conjugates in which a sortilin binding moiety is linked, directly or indirectly, with a payload moiety. In some embodiments, a sortilin binding moiety specifically binds to a surface factor on target cells of interest (e.g., on sortilin-expressing cells such as sortilin-expressing cancer cells). In some embodiments, a payload moiety is or comprises a therapeutic agent (e.g., an anti-cancer agent) or a detectable agent (e.g., a diagnostic agent).

Among other things, the present disclosure provides an insight that sortilin binding agents as described herein may be particularly useful or effective for the delivery of therapeutic agents to cancer cells and/or to other cells that express or otherwise comprise a surface factor specifically bound by a sortilin binding moiety as described herein. For example, the present disclosure provides technologies that achieve delivery of a payload to sortilin expressing cells (e.g., that display sortilin on their surfaces) by contacting the cells with a conjugate as described herein.

### Sortilin

Sortilin (SORT 1) belongs to the Vps10p domain sorting receptor family composed of heterogeneous type-1 receptors that are broadly expressed in mammalian tissues, particularly in the brain (see, for example, Petersen et al., 1999). For example, sortilin has been described as being highly expressed in brain, heart, skeletal muscle, and spinal cord, and also expressed, albeit at lower levels, in kidney, liver, pancreas, small intestine, and spleen (see, for example, Petersen et al., J Biol Chem 6:3599, 1997); sortilin is also expressed in immune cells (see, for example, Patel et al., Circ Res 116:789, 2015; Mortensen et al., J Clin Invest 124:5317, 2014). In the brain, sortilin has been reported to be predominantly expressed in neurons, with some cell-type and regional variability (see, Xu et al., Front Neuroanat. 13:31, 2021).

Sortilin has been reported to function as a sorting and trafficking receptor, shuttling proteins between the cell surface and various intracellular compartments (see, for example, Ouyang, et al. J. Cell Physiol. 235:8958, 2020). Dysregulation of sortilin has been reported to contribute to the development of various diseases, including cancers, certain neurological diseases (e.g., Alzheimer's disease, Parkinson's disease), cardiovascular disease, and certain metabolic diseases (e.g., type II diabetes mellitus (reviewed, for example, in Al-Yozbaki et al., Rev. on Cancer 1874:188429, 2020).

For example, sortilin has been reported to mediate beta-secretase trafficking, thereby increasing beta-amyloid precursor protein cleavage, relevant to development of Alzheimer's disease (see, for example, Walter et al., Curr Opin Neurobiol 11:585, 2001). Also, sortilin levels have been reported to correlate with levels of circulatory low density lipoprotein, a hallmark of cardiovascular disease (see, for example, Gustafsen et al., Cell Metab. 19:310, 2014). Furthermore, there have been numerous compelling reports of sortilin overexpression in cancer cells. Its clinicopathological significance in oncology has been reported in various types of human cancers, including neuroendocrine (see, for example, Kim et al., 2018; Rhost et al., 2018; Roselli et al., 2015), breast (see, for example, Rhost, S., Breast Cancer Res, 2018; Berger, BMC Cancer, 2021), pancreatic (see, for example, Gao, Am J Path, 2020), colorectal (see, for example, Akil et al., 2011), ovarian (see, for example, Ghaemimanesh et al., 2014; Hemmati et al., 2009), and hematological malignancies (e.g., chronic lymphocytic leukemia [CLL]; see, for example, Lia Farahi et al., 2019).

Sortilin has a large extracellular domain, and a short intracellular domain, sometimes referred to as its cytoplasmic tail (see, for example, Petersen et al., J Biol Chem 272:3599, 1997). The extracellular domain includes a ten bladed β-propeller domain N-terminal to two small domains, 10CC-a and 10CC-b, representing ten conserved cysteines (10CC), which interact with the β-propeller domain (see Quistgaard & Thirup BMC Struct Biol 9:46, 2009).

Sortilin is predominantly monomeric at neutral pH and predominantly a dimer at acidic pH. Many ligands display high affinity for (monomeric) sortilin at neutral pH and reduced affinity for (dimeric) sortilin at acidic pH; it has been proposed that dimerization disrupts certain ligand binding site(s); and furthermore that impact dimerization, and/or of pH, may help sortilin achieve differential trafficking of different ligands (see, for example, Mitok et al., J Lipid Res 63:100243, and references cited therein).

More than fifty different agents have been reported to bind to sortilin and/or to be trafficked via a mechanism that is impacted by sortilin activity (see Table 1 in Mitok et al., J Lipid Res 63:100243, 2022). Certain competitive binding studies have suggested that different sortilin ligands may bind to distinct, though potentially partially overlapping, sites on sortilin (see, for example, Quistgaard, et al., Nat. Struct. Mol. Biol. 16:96, 2009; Trabjerg, et al, Structure. 27:1103, 2019; Serup et al., J. Biol. Chem. 285: 12210, 2010. Moreover, it has been established that different ligands can have different impacts on sortilin, including different conformational changes (see, Trajberg et al., Structure 27:1103, 2019).

### Progranulin

One sortilin ligand of particular relevance to the present disclosure is progranulin.

Progranulin is a highly conserved secreted protein that is expressed in multiple cell types, including in both CNS and peripheral tissues. Progranulin is initially produced and secreted as a glycosylated protein that becomes cleaved into 6 kDa peptides known as granulins A-G (reviewed in Townley et al., Neurology 90:118, 2018). Progranulin, and/or granulins generated from it, has been reported to be involved in various biological pathways, including cell growth, survival, repair, and inflammation. Specifically, each of progranulin and various granulins has been demonstrated to activate or repress genes involved in transcription, splicing, stress response, endosomal sorting, cytoskeleton maintenance, proteostasis, etc. (see Rollinson et al. Eur J Neurosci 44:2214, 2016. Progranulin has also been implicated in promoting tumorigenesis and/or metastasis (see, for example, Berge et al., BMC Cancer 21:185, 2021).

Studies have demonstrated that sortilin regulates progranulin trafficking, and is a main determinant of progranulin level in the brain.

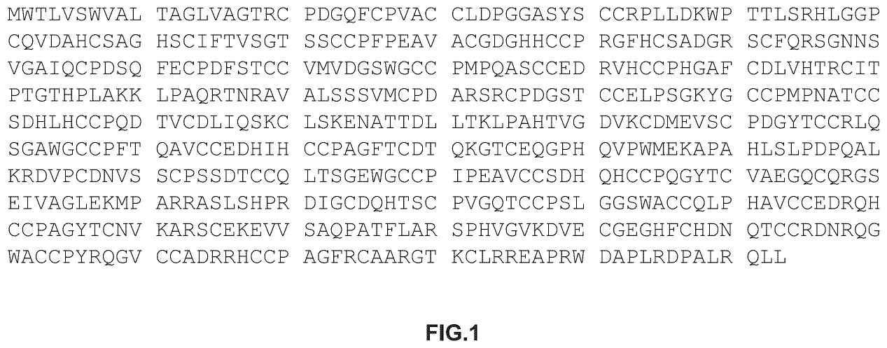

The amino acid sequence of human progranulin is provided in FIG. 1. It is the C-terminal portion of progranulin that binds to sortilin (and specifically to the propeller region of sortilin). Indeed, the C-terminal progranulin fragment is fully sufficient for sortilin binding, and will displace sortilin-bound full-length progranulin.

Specifically, Zheng at al have reported that the last 24aa of progranulin (C24, aa 570-593) is fully sufficient for binding to sortilin. The last 6 residues (C6, ALRQLL (SEQ ID NO: 25)) are able to mediate progranulin-sortilin interaction, although not to the same extent as the 24 residues. Moreover, Zheng et al, found that addition of residues to the progranulin C-terminus (+7aa) abolishes the binding of progranulin to sortilin, further confirming a critical role of progranulin's C-terminal leucine residue in mediating its binding to sortilin (Zheng et al, 2011). Deletion of the last 3 residues of progranulin (QLL) abolishes its binding to sortilin and also sortilin dependent regulation of progranulin trafficking. While Zheng et al have reported specific sequences that are sufficient for binding to sortilin, Zheng et al is silent regarding sortilin binding agents (e.g., a sortilin binding moiety (e.g., a sortilin binding peptide as described herein) that may be conjugated to or otherwise associated with one or more additional moieties) as described herein.

Progranulin has been described as a “key mediator involved in breast cancer progression” (see Berger et al., BMC Cancer 21:185, 2021), and sortilin-mediated endocytosis of progranulin has been reported to contribute to, and indeed to be required for, progranulin to induce metastasis of breast cancer cells. (Rhost et al, Breast Cancer Res. 20:137, 2018). Sortilin is highly expressed in breast cancer cell lines as compared with non-tumorigenic breast epithelial cells (see Berger BMC Cancer 2021), and researchers have suggested that co-expression of progranulin and sortilin may be an effective biomarker for identification of a highly malignant subtype of breast cancer, which may be responsive to therapy with agents that target the sortilin-progranulin interaction.

### Sortilin Binding Agents

The present disclosure provides certain sortilin binding agents and compositions (e.g., conjugates) that include them.

Provided sortilin binding agents are or comprise a sortilin binding moiety as described herein, and optionally include one or more other moieties (e.g., one or more linker moieties and/or one or more payload moieties). In some embodiments, a sortilin binding agent includes a sortilin binding moiety (e.g., a sortilin binding peptide as described herein) that is conjugated or otherwise associated with one or more additional moieties (e.g., one or more linker moieties and/or one or more payload moieties), but in some embodiments, an unconjugated sortilin binding agent (e.g., sortilin binding peptide) may be utilized.

In some embodiments, a provided sortilin binding agent is characterized by one or more functional or performance attributes, for example as discussed below. In some such embodiments, such functional or performance attribute is assessed on a sortilin binding moiety or agent absent a payload; in some such embodiments, such functional or performance attribute is assessed for a conjugate comprising a sortilin binding moiety, a payload and, optionally, a linker.

In some embodiments, a provided sortilin binding agent (or sortilin-binding moiety thereof) is characterized by its ability to bind to sortilin.

In some embodiments, a provided sortilin binding agent (e.g., a sortilin-binding moiety) binds to sortilin with an affinity up to 1 μM and/or with an affinity reasonably comparable to or greater than that of progranulin and/or to that of a C-terminal fragment of progranulin (e.g., to a peptide that is or includes about 15-20, or specifically 17 C-terminal residues of progranulin).

In some embodiments, affinity may be assessed, for example, by fluorescence polarization; in some such embodiments, a provided sortilin binding agent (e.g., a sortilin-binding moiety) shows an affinity for sortilin when assessed by fluorescence polarization that is not more than about 500 nM, about 450 nM, about 400 nM, about 300 nM, about 200 nM, about 100 nM, about 50 nM, about 40 nM, about 35 nM, or less.

In some embodiments, affinity may be assessed, for example, by grating-coupled interferometry (GCI); in some such embodiments, a provided sortilin binding agent (e.g., a sortilin-binding moiety) shows an affinity for sortilin when assessed by GCI that is not more than about 1 μM, for example not more than about 500 nM, about 450 nM, about 400 nM, about 300 nM, about 200 nM, about 100 nM, about 50 nM or less.

In some embodiments, a provided sortilin binding agent (e.g., a sortilin-binding moiety) is characterized by an ability to compete with progranulin and/or with a C-terminal fragment of progranulin (e.g., with a peptide that is or includes about 15-20, or specifically 17 C-terminal residues of progranulin) for binding to sortilin. For example, in some embodiments, a provided sortilin binding agent (e.g., a sortilin-binding moiety) shows an IC50 less than about 12 μM, about 11 μM, about 10 μM, about 9 μM, about 8 μM, about 7 μM, about 6 μM, about 5 μM, about 4 μM, about 3 μM, about 2 μM, about 1 μM, about 900 nM, about 800 nM, about 750 nM, about 700 nM, about 650 nM, about 600 nM, about 550 nM, about 500 nM, about 450 nM, about 400 nM, about 350 nM, about 300 nM, or less, for example below about 300-350 nM or about 300-400 nM, or about 400-600 nM, or about 600-800 nM, or about 600-700 nM, or about 600-650 nM. In some particular embodiments, a provided sortilin binding agent that is or comprises a cyclic peptide sortilin binding agent is characterized by an IC50 in such an assay that is within a range of about 500 nM or below to about 12 μM, or about 500 μM or below to about 5 μM or below about 4 μM, about 3 μM, about 2 μM, about 1 μM, or less (e.g., below about 600-650 nM). In some particular embodiments, a provided sortilin binding agent that is or comprises a linear peptide sortilin binding agent is characterized by an IC50 in such an assay that is within a range of about 300 nM or below to about 10 μM, or about 300 nM or below to about 4 μM or about 300 nM or below to about 3 μM or about 300 nM or below to about 2 μM or about 300 nM or below to about 1 μM or about 300 nM or below to about 700 nM or about 300 nM or below to about 600 nM or about 300 nM or below to about 500 nM or about 300 nm or below to about 400 nM or about 300 nM or below to about 350 nM, such as below about 300-400 nM or below about 300-350 nM.

In some embodiments, a provided sortilin binding agent (e.g., a sortilin-binding moiety) is characterized by stability, for example in a particular environment and/or under certain conditions (e.g., in serum, in storage, etc). In some embodiments, the present disclosure provides compositions or preparations of a sortilin binding agent in such environment or under such conditions and/or that has been exposed thereto and/or stored therein (e.g., for a particular period of time).

In some embodiments, a provided sortilin binding agent (e.g., a sortilin-binding moiety) is characterized by stability in a particular environment and/or under certain conditions that is comparable to or better than that of a progranulin or a fragment thereof (e.g., a C-terminal fragment of progranulin, such as a peptide that is or includes about 15-20, or specifically 17 C-terminal residues of progranulin). In some particular embodiments, such stability is in serum (e.g., in mammalian serum such as in murine serum or human serum).

In some embodiments, a provided sortilin binding agent (e.g., a sortilin-binding moiety) is characterized by a half life of at least about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 25 minutes, about 30 minutes, about 35 minutes, about 40 minutes, about 45 minutes, about 50 minutes about 60 minutes, about 70 minutes, about 80 minutes, about 90 minutes, about 100 minutes, about 110 minutes, about 120 minutes, about 130 minutes, about 140 minutes, about 150 minutes, about 160 minutes, about 170 minutes or more in serum (e.g., in mammalian serum such as in murine serum and/or in human serum). Among other things, the present disclosure teaches that cyclic peptides, e.g., as provided herein, may often have longer half lives.

In some embodiments, a provided sortilin binding agent (e.g., a sortilin-binding moiety) is characterized by a half life that is not more than a particular period of time. That is, among other things, the present disclosure appreciates that, in some contexts (e.g., as will be recognized by those skilled in the art reading the present disclosure), it may be desirable to utilize an agent with a shorter half life, for example less than about 10 minutes, about 9 minutes, about 8 minutes, about 7 minutes, about 6 minutes, about 5 minutes, about 4 minutes, about 3 minutes, or less.

In some embodiments, it may be desirable to utilize a provided sortilin binding agent with a half life in serum that is reasonably comparable to that of one or more endogenous ligands of sortilin (e.g., of progranulin).

In some embodiments, a provided sortilin binding agent comprises a sortilin binding moiety wherein at least one amino acid residue is conjugated with a payload moiety. In some embodiments, a provided sortilin binding agent comprises a sortilin binding moiety wherein two or more amino acid residue are conjugated with a payload moiety. In some embodiments, at least one amino acid residue within the sequence outside of P15, P16 and P17 of sortilin binding moiety is conjugated with a payload moiety. In some embodiments, two or more amino acid residue within the sequence outside of P15, P16 and P17 of sortilin binding moiety are conjugated with a payload moiety. In some embodiments, a provided sortilin binding agent comprises a linear sortilin binding moiety comprising at least one lysine residue which is conjugated with a payload moiety. In some embodiments, a provided sortilin binding agent comprises a linear sortilin binding moiety wherein any lysine residue within the sequence is conjugated with a payload moiety. In some embodiments, a sortilin binding moiety is conjugated to a payload moiety through a linker moiety.

In some embodiments, a provided sortilin binding agent comprises a sortilin binding moiety wherein at least one amino acid residue is conjugated with a linker and a payload moiety at a specific site. In some embodiments, a provided sortilin binding moiety can be characterized by the sequence pattern for cyclic group 1 as disclosed in this disclosure, wherein the amino acid residue at P3 site is conjugated with a linker and a payload moiety. In some embodiments, a provided sortilin binding moiety can be characterized by the sequence pattern for cyclic group 1 as disclosed in this disclosure, wherein the amino acid residue at P8 site is conjugated with a linker and a payload moiety. In some embodiments, a provided sortilin binding moiety can be characterized by the sequence pattern for cyclic group 2 as disclosed in this disclosure, wherein the amino acid residue at P10 site is conjugated with a linker and a payload moiety. In some embodiments, a provided sortilin binding moiety can be characterized by the sequence pattern for any one of cyclic group 1 or cyclic group 2 as disclosed in this disclosure, wherein the amino acid residue at P4 site is conjugated with a linker and a payload moiety. In some embodiments, a provided sortilin binding moiety can be conjugated with a linker and a payload at two or more sites.

### Sortilin Binding Moiety

Among other things, the present disclosure provides an insight that peptide agents corresponding to a fragment of progranulin, or of a variant thereof, may be particularly useful as sortilin binding agents or moieties. In particular, the present disclosure provides an insight that peptide agents corresponding to a C-terminal fragment of progranulin, or a variant thereof, are particularly useful.

In some embodiments, a C-terminal fragment of progranulin includes the progranulin C-terminus. In some embodiments, a C-terminal fragment of progranulin includes about 12, about 13, about 14, about 15, about 16, about 17, about 18, about 19, or about 20 C-terminal residues of progranulin. In some embodiments, a C-terminal fragment of progranulin includes a C-terminal “QLL” motif.

In some embodiments, a peptide variant of progranulin, or of a fragment (e.g., a C-terminal fragment) thereof, is a peptide whose amino acid sequence corresponds substantially to that of progranulin (or to the relevant fragment, such as a C-terminal fragment, thereof) but includes at least amino acid substitution, addition, or deletion relative that of progranulin (or of the relevant fragment thereof). In many embodiments, a peptide variant of progranulin has an amino acid sequence that corresponds substantially to that of the relevant portion of progranulin but for one or more amino acid substitutions. Those skilled in the art will appreciate that a peptide variant of progranulin, or of a fragment (e.g., a C-terminal fragment) thereof, includes sufficient amino acid identity or similarity (e.g., substitution of homologous residues) to be recognizable as related to progranulin. For example, in many embodiments, a peptide variant of progranulin has an amino acid sequence that corresponds to a continuous stretch of about 10 or more (e.g., about 10, about 11, about 12, about 13, about 14, about 15, about 16, about 17, about 18, about 19, or about 20 contiguous residues of progranulin) and includes 1, 2, 3, 4, or more amino acid substitutions relative to such contiguous stretch of progranulin.

The present disclosure provides a particular insight that non-natural amino acids may be particularly useful for inclusion in sortilin binding peptides as described herein.

The present disclosure further provides an insight that linear peptides, for example having a length within a range of about 12 to about 20 amino acids that show at least about 60%, 65%, 70%, 75% or more sequence identity with a fragment of progranulin (e.g., with a C-terminal fragment thereof) may be particularly useful in certain embodiments.

The present disclosure further provides an insight that certain cyclic peptides as described herein which, in many embodiments, include at least one, and often a plurality of non-natural amino acids and alternatively or additionally include at least one sequence feature found in a fragment (e.g., a C-terminal fragment, such as one including at least about 12, about 13, about 14, about 15, about 16, or about 17 C-terminal residues) of progranulin may be particularly useful.

The present disclosure further provides particular insights that, in some embodiments it may be desirable to include in a peptide sortilin binding moiety (i) a basic residue (e.g., a specific such residue such as L-arginine or an analog thereof as exemplified in Table 3) at a position corresponding to position P3 and/or P4 of a 17mer C-terminal fragment of progranulin; (ii) a hydrophobic residue (e.g., a specific such residue such as a L-tryptophan or an analog thereof as exemplified in Table 4) at a position corresponding to position P4 of a 17mer C-terminal fragment of progranulin; (iii) a long hydrophobic residue (e.g., a specific such residue as exemplified Table 5) at a position corresponding to position P10 a 17mer C-terminal fragment of progranulin; (iv) a covalent bond (e.g., a disulfide bond) between residues (e.g., between cysteine residues or analogs thereof) between position corresponding to positions P5 and P13 or P8 and P12 of a 17mer C-terminal fragment of progranulin; and/or (v) a hydrophobic residue (e.g., a specific such residue, such as Leucine or a Leucine analog, for example as exemplified in Table 7) at a position corresponding to position P16 and/or P17 of a 17mer C-terminal fragment of progranulin.

In some embodiments, a provided sortilin binding agent or moiety is or comprises a peptide which has a length within a range of about 12 to about 20 amino acids and includes a characteristic sequence represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein X is any canonical or non-canonical amino acid.

As described in various embodiments in reference to a characteristic sequence described herein, each instance of “X” is independently any canonical or non-canonical amino acid. In some embodiments, each X is independently selected from a canonical or non-canonical amino acid in Tables 2-7 herein. As also described herein, “Xn” (e.g., X4, X5, X6, etc.), refers to an amino acid “X” at site “n” within a characteristic sequence. For example, X4 refers to an amino acid that is the fourth residue in the characteristic sequence, X5 refers to an amino acid that is the fifth residue in the characteristic sequence, etc. Reference to X^{m-n} in a given sequence is intended to refer to m-n instances of X, wherein each instance of X is independently selected from any canonical or non-canonical amino acid. For example, “X²⁻³” is intended to refer to 2 or 3 instances of X within the characteristic sequence, wherein each instance of X is independently selected from any canonical or non-canonical amino acid. X⁰ is intended to indicate that no amino acid is present. For example, C X⁰ R is intended to refer to a sequence that is just C bound directly to R.

Moreover, as described in various embodiments herein, two or more amino acids within parentheses and separated by a slash “/” are intended to refer to amino acids at the given position in the alternative. For example, (R/N) is intended to refer, at that position, to either be amino acid R or amino acid N.

In some embodiments, a provided sortilin binding agent or moiety corresponds to a C-terminal fragment of progranulin, or a variant thereof, and includes not more than about 20 contiguous residues corresponding to contiguous progranulin residues.

In some embodiments, a provided sortilin binding moiety is or comprises a cyclic peptide. In some embodiments, a provided sortilin binding moiety includes a characteristic sequence which is represented by:

(SEQ ID NO: 3)
(R/N) X²⁻³ C X⁰⁻¹ R (Q/E)

In some embodiments, a provided sortilin binding moiety is or comprises a linear peptide. In some embodiments, a provided sortilin binding moiety is or comprises a linear peptide. In some embodiments, a provided sortilin binding moiety includes a characteristic sequence represented by SEQ ID NO:2. In some embodiments, a provided sortilin binding moiety includes a the characteristic sequence represented by:

(SEQ ID NO: 4)
(R/N) X²⁻³ L X⁰⁻¹ R (Q/B43/B50).

In some embodiments, a provided sortilin binding moiety includes a characteristic sequence represented by:

(SEQ ID NO: 5)
(R/N) X²⁻³ C/L X⁰⁻¹ R Q(L/B13/F02)(L/B13/F02).

In some embodiments, a provided sortilin binding moiety includes a characteristic sequence represented by:

(SEQ ID NO: 6)
(R/N) X²⁻³ CX⁰⁻¹ R Q(L/B13/F02)(L/B13/F02).

In some embodiments, a provided sortilin binding moiety includes a characteristic sequence according to any one of SEQ ID NO: 1, 3, 5 or 6, wherein the characteristic sequence includes at least a second cysteine residue and the polypeptide includes at least one disulfide bond between cysteine residues.

In some embodiments, a provided sortilin binding moiety includes a characteristic sequence represented by:

(SEQ ID NO: 7)
(R/N) X10 X11 X12 C R Q.

In some embodiments, a provided sortilin binding moiety includes a characteristic sequence represented by:

(SEQ ID NO: 8)
X4 X5 X6 X7 X8 (R/N) X10 X11 X12 C R Q;

wherein at least one of X4, X5, and X6 is a cysteine residue. In some embodiments, a provided sortilin-binding polypeptide includes at least one disulfide bond between cysteine residues.

In some embodiments, a provided sortilin binding moiety includes a characteristic sequence represented by:

(SEQ ID NO: 9)
(R/E) X4 X5 X6 X7 X8 (R/N) X10 X11 X12 C R Q;

wherein at least one of X4, X5, and X6 is a cysteine residue. In some embodiments, a provided sortilin binding polypeptide includes at least one disulfide bond between cysteine residues.

In some embodiments, a provided sortilin binding moiety includes a characteristic sequence represented by:
(R/E) X4 X5 X6 X7 X8 (R/N) X10 X11 X12 CR Q (L/B13/F02) (L/B13/F02) (SEQ ID NO: 10);
wherein at least one of X4, X5, and X6 is a cysteine residue. In some embodiments, a sortilin binding polypeptide includes at least one disulfide bond between cysteine residues.

In some embodiments, a provided sortilin binding moiety includes a characteristic sequence represented by:

(SEQ ID NO: 11)
C R X10 X11 C X13 R Q;

wherein the two cysteine residues form a disulfide bond and the polypeptide is a cyclic polypeptide.

In some embodiments, a provided sortilin binding moiety includes a characteristic sequence represented by:

(SEQ ID NO: 12)
(R/H) N X6 X7 C R X10 X11 C X13 R Q;

wherein the two cysteine residues form a disulfide bond and the polypeptide is a cyclic polypeptide.

In some embodiments, a provided sortilin binding moiety includes a characteristic sequence represented by:

(SEQ ID NO: 13)
(R/H) N X6 X7 CR X10 X11 CX13 R Q (L/B13/F02)
L/B13/F02);

wherein the two cysteine residues form a disulfide bond and the polypeptide is a cyclic polypeptide.

In some embodiments, a provided sortilin binding moiety has XⁿRDPALRXLL sequence (SEQ ID NO: 14), wherein X is any canonical or non-canonical amino acid according to current disclosure, and n is 0, 1, 2, 3, 4, 5, 6, 7 or 8. In some embodiments, for example, a peptide compound according to current disclosure has RDPALRQLL sequence (SEQ ID NO: 21). In some embodiments, for example, a peptide compound according to current disclosure has RDPALR(B43)LL sequence (SEQ ID NO: 22). In some embodiment, RDPALR(B43)LL (SEQ ID NO: 22) shows reduced affinity compared to APRWDAPLRDPALR(B43)LL (SEQ ID NO: 23).

In some embodiments, a provided sortilin binding moiety includes one or more of:
(i) a basic residue such as L-arginine or an analog thereof at a position corresponding to position P3 and/or P4 of a 17mer C-terminal fragment of progranulin;
(ii) a hydrophobic residue such such as a L-tryptophan or an analog thereof at a position corresponding to position P4 of a 17mer C-terminal fragment of progranulin;
(iii) a long hydrophobic residue at a position corresponding to position P10 a 17mer C-terminal fragment of progranulin;
(iv) a covalent bond such as a disulfide bond) between residues at positions corresponding to positions P5 and P13 or P8 and P12 of a 17mer C-terminal fragment of progranulin; and
(v) a hydrophobic residue such as Leucine or a Leucine at a position corresponding to position P16 and/or P17 of a 17mer C-terminal fragment of progranulin.

In some embodiments, X represents an amino acid included in one or more of Tables 2-7.

In some embodiments, X represents a canonical amino acid. In some embodiments, X is a non-canonical amino acid. In some embodiments, a non-canonical amino acid is selected from those in one or more of Tables 2-7.

In some embodiments, a provided sortilin binding agent or moiety is or comprises a peptide which has a length within a range of about 12 to about 20 amino acids and includes a characteristic sequence represented by:

(SEQ ID NO: 15)
(R/N) X²⁻³ (C/L) X⁰⁻¹ R Z;

wherein each X is independently any canonical or non-canonical amino acid. In some embodiments, X represents a canonical amino acid. In some embodiments, X is a non-canonical amino acid. In some embodiments, a non-canonical amino acid is selected from those in one or more of Tables 2-7. In some embodiments, Z represents a canonical amino acid. In some embodiments, Z represents a polar amino acid. In some embodiments, Z represents an amino acid comprising a side chain with an acidic group. In some embodiments, side chain of Z comprises —COOH. In some embodiments, Z is Q, E or an analog thereof. In some such embodiments, an analog of Q may be, for example (but not limited to), Theanine, 2,4-diaminopentanedioic acid, D-glutamine, Na-acetyl-L-glutamine, 5-methylglutamine, 2-methyl-L-glutamine, etc. In some embodiments, Z is L or an analog thereof. In some such embodiments, an analog of L is selected from table 7. In some embodiments, Z represents a non-canonical amino acid selected from those in one or more of Tables 2-7. In some embodiments, Z may be, for example (but not limited to), D-leucine, L-3-cyclopropyl-alanine, L-4-methyl-leucine, L-homoleucine, 3-cyclobutyl-alanine, L-Norleucine, L-3-methyl-valine, (2S,4R)-2-amino-4-hydroxypentanoic acid, (2R,4S)-2-amino-4-hydroxypentanoic acid, (4S)-4-hydroxy-L-Norvaline, (4R)-4-Hydroxy-D-Norvaline, 5-Methyl-D-Norleucine, L-homoserine or beta-alanine.

In some embodiments, a provided sortilin binding agent or moiety is or comprises a peptide having an amino acid sequence APRWDAPLRDPALRQLL (SEQ ID NO: 29). In some embodiments, a provided sortilin binding agent or moiety is or comprises a peptide having an amino acid sequence APRWDAPLRDPALRQ(B13)(G48) (SEQ ID NO: 30).

In some embodiments, a provided sortilin binding agent or moiety is characterized in that:
(i) it demonstrates affinity (Kd) for human sortilin 1 below about 1 μM when assessed by fluorescence polarization;
(ii) in a competitive binding assay with a reference C-terminal progranulin fragment, it demonstrates an IC₅₀ less than about 12 μM;
(iii) when maintained under murine serum, it displays stability greater than that of the reference C-terminal progranulin fragment.

In some embodiments, a provided sortilin binding agent or moiety is characterized in that:
(i) it demonstrates affinity (Kd) for human sortilin 1 below about 1 μM when assessed by fluorescence polarization;
(ii) in a competitive binding assay with a reference C-terminal progranulin fragment, it demonstrates an IC₅₀ less than about 12 μM;
(iii) when maintained under murine serum, it displays stability lower than that of the reference C-terminal progranulin fragment.

In some embodiments, the reference C-terminal progranulin fragment has an amino acid sequence that is or comprises: APRWDAPLRDPALRQLL (SEQ ID NO: 29). In some embodiments, a provided sortilin binding agent or moiety has an amino acid sequence that is or comprises DDPRAPWPALQRLALRL (SEQ ID NO: 34).

In some embodiments, a provided sortilin binding agent or moiety is characterized by a stability half life in murine serum that is greater than about >3 minutes. In some embodiments, stability half life in murine serum is about 5 minutes. In some embodiments, stability half life in murine serum is about 10 minutes. In some embodiments, stability half life in murine serum is about 14 minutes.

In some embodiments, a provided sortilin binding agent or moiety is characterized by an IC50 in the competitive binding assay that is less than about 5000 nM. In some embodiments, IC50 is less than about 4000 nM. In some embodiments, IC50 is less than about 3000 nM.

In some embodiments, IC50 is less than about 2000 nM. In some embodiments, IC50 is less than about 1000 nM. In some embodiments, IC50 is less than about 750 nM. In some embodiments, IC50 is about 600-650 nM. In some embodiments, IC50 is less than about 600 nM. In some embodiments, the IC50 is less than about 500 nM. In some embodiments, IC50 is less than about 400 nM. In some embodiments, IC50 is less than about 300-400 nM. In some embodiments, IC50 is less than about 300-350 nM.

### Linker

As noted above, present disclosure provides conjugates comprising (a) a polypeptide; and (b) a payload; and (c) optionally, a linker, wherein the polypeptide includes a sortilin binding moiety.

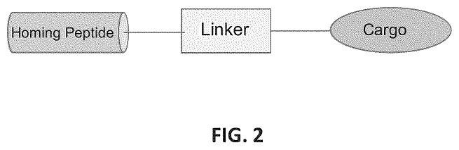

In some embodiments, a conjugate agent has the structure of Formula I, depicted in FIG. 2; wherein each of the homing peptide, linker, and payload is as defined above and described herein.

Those skilled in the art will be aware of various types of linkers, and chemical compositions thereof, that can be utilized to associate one or more payloads with one or more sortilin binding moieties as described herein.

Generally, linkers are designated as “cleavable” or “non-cleavable”. Cleavable linkers are typically employed when it is desired that the payload and binding moiety to which it is conjugated be released (e.g., when a conjugate encounters a particular environment or condition, such as a particular pH or a particular enzyme, such as a particular protease).

In some embodiments, a cleavable linker is cleaved chemically, for example by hydrolysis, change in pH, reduction or oxidation. In some embodiments, a cleavable linker is cleaved enzymatically, for example by action of a protease, an esterase, a glucosidase, a glucuronidase, galactosidase, a phosphatase, phosphodiesterase, nuclease, lipase or any enzyme that is capable of cleaving the relevant linker.

In some embodiments, a cleavable linker is or comprises a disulfide linkage, an ester, a phosphodiester, a saccharide, or a lipid.

In some embodiments, a non-cleavable linker is chemically, enzymatically, or otherwise biochemically and physiologically stable. As such, a non-cleavable linker typically lacks linkages that are chemically, biochemically, enzymatically cleavable or are otherwise physiologically unstable.

In some embodiments, a linker is multivalent (e.g., so that it can or does link multiple payload moieties to a sortilin binding moiety, or multiple sortilin binding moieties to a payload moiety, or both). In some such embodiments, a linker is bivalent, trivalent, etc.

In some embodiments, a linker is or comprises a bivalent straight or branched C1-40 aliphatic chain, wherein one or more methylene units of an aliphatic chain is/are replaced by a group selected from —CH(R)—, —C(R)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur,
wherein:
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, a linker is or comprises a bivalent straight or branched C₁₋₃₅ aliphatic chain, wherein one or more methylene units of the aliphatic chain are replaced by a group selected from —CH(R)—, —C(R)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(—O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur,
wherein:
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, a linker is or comprises a bivalent straight or branched C₁₋₃₀ aliphatic chain, wherein one or more methylene units of the aliphatic chain are replaced by a group selected from —CH(R)—, —C(R)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(—O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur,
wherein:
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, a linker is or comprises a bivalent straight or branched C₁₋₂₅ aliphatic chain, wherein one or more methylene units of the aliphatic chain are replaced by a group selected from —CH(R)—, —C(R)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur,
wherein:
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, a linker is or comprises a bivalent straight or branched C₁₋₂₀ aliphatic chain, wherein one or more methylene units of the aliphatic chain are replaced by a group selected from —CH(R)—, —C(R)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur,
wherein:
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, a linker is or comprises a bivalent straight or branched C₁₋₁₅ aliphatic chain, wherein one or more methylene units of the aliphatic chain are replaced by a group selected from —CH(R)—, —C(R)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur,
wherein:
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, a linker is or comprises a bivalent straight or branched C₁₋₁₀ aliphatic chain, wherein one or more methylene units of the aliphatic chain are replaced by a group selected from —CH(R)—, —C(R)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(—O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur,
wherein:
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, a linker is or comprises a bivalent straight or branched C₁₋₅ aliphatic chain, wherein one or more methylene units of the aliphatic chain are replaced by a group selected from —CH(R)—, —C(R)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur,
wherein:
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, a linker is or comprises a structure selected from

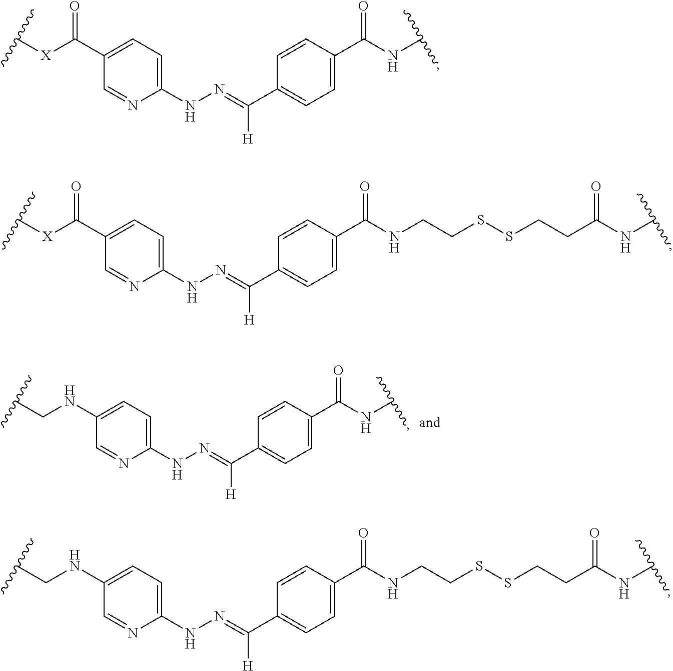

wherein X is NH or O.

In some embodiments, a cleavable linker is a cathepsin-cleavable linker. In some such embodiments, a linker is or comprises a valine-citrulline (Val-Cit) motif:

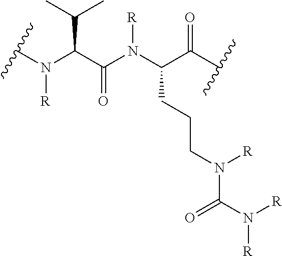

wherein R is hydrogen or C₁₋₆ aliphatic.

In some embodiments, a valine-citrulline linker is or comprises

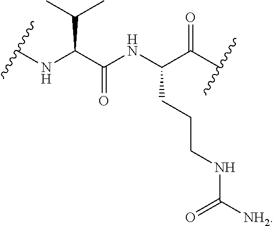 [SMILES: *N[C@H](C(=O)N[C@@H](CCCNC(N)=O)C(*)=O)C(C)C]

In some embodiments, a valine-citrulline linker is or comprises

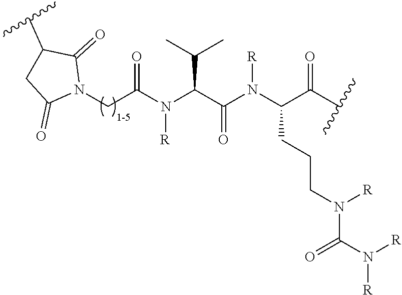

wherein R is hydrogen or C₁₋₆ aliphatic.

In some embodiments, a valine-citrulline linker is or comprises

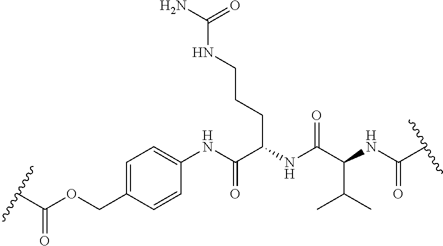 [SMILES: *C(=O)N[C@H](C(=O)N[C@@H](CCCNC(N)=O)C(=O)Nc1ccc(COC(*)=O)cc1)C(C)C]

In some embodiments, a valine-citrulline linker is or comprises:

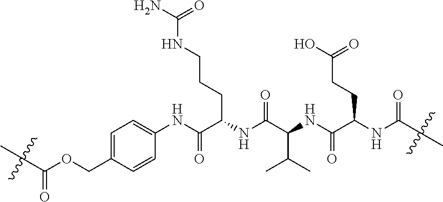 [SMILES: *C(=O)N[C@H](CCC(=O)O)C(=O)N[C@H](C(=O)N[C@@H](CCCNC(N)=O)C(=O)Nc1ccc(COC(*)=O)cc1)C(C)C]

In some embodiments, a linker comprises a disulfide linkage. In some embodiments, the linker comprises a poly(ethyleneglycol) moiety (e.g., —(CH₂CH₂O)_{b}—), wherein b is 1-50.

In some embodiments, a linker is or comprises a group selected from

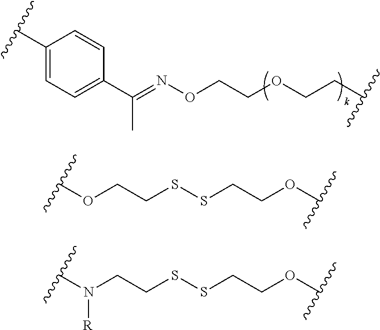

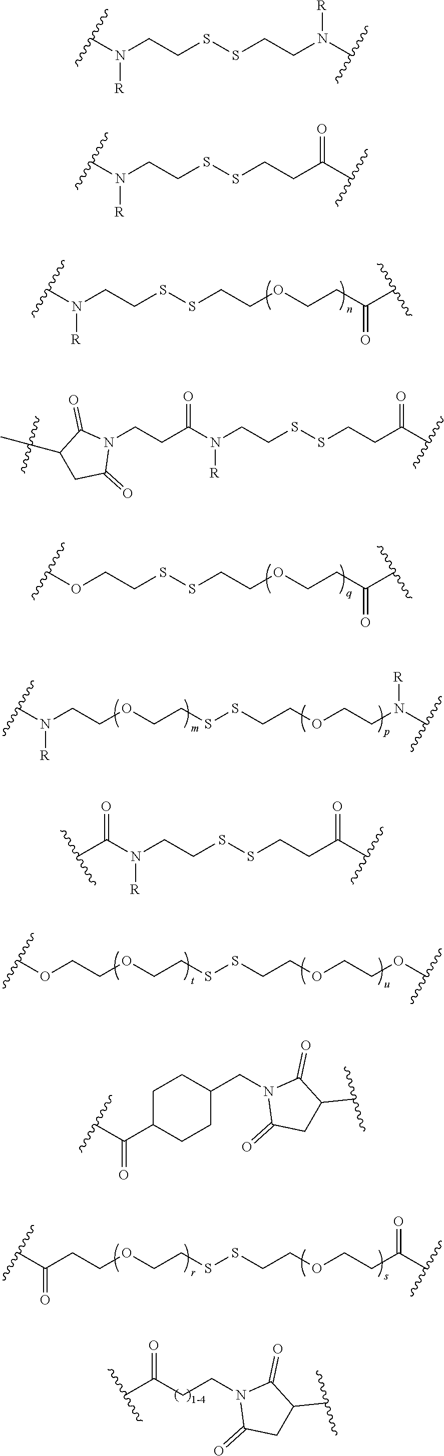

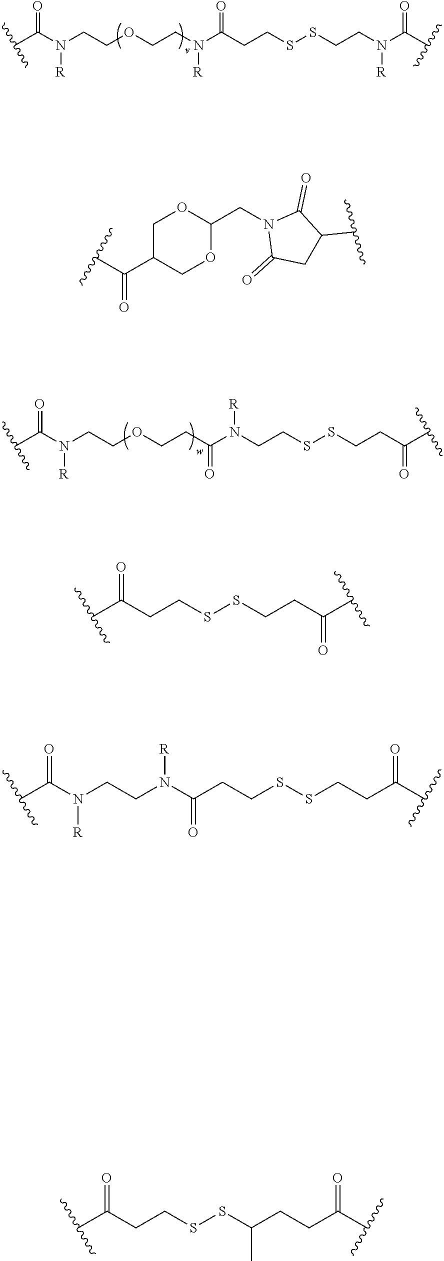

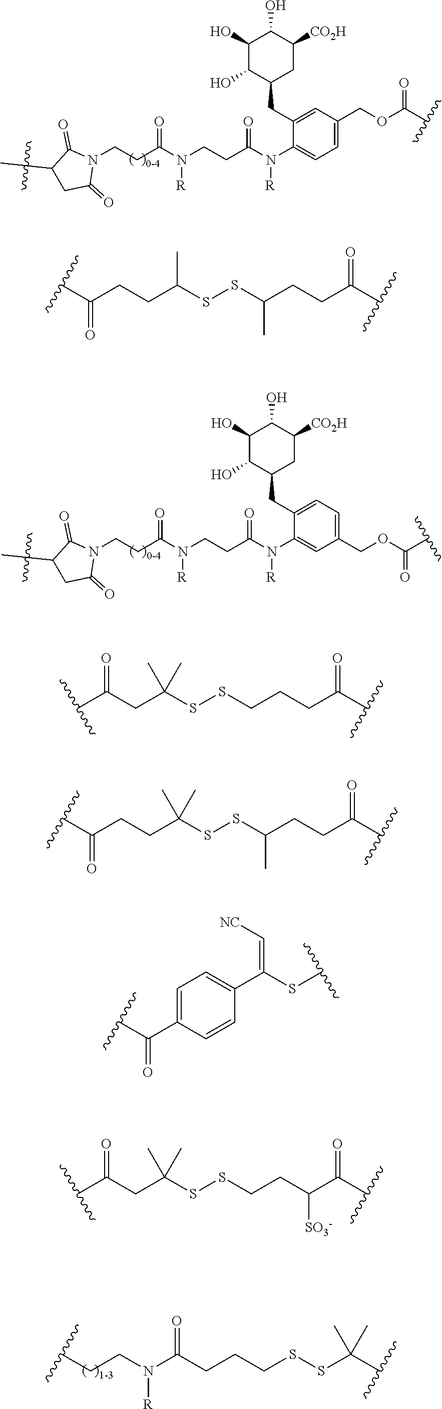

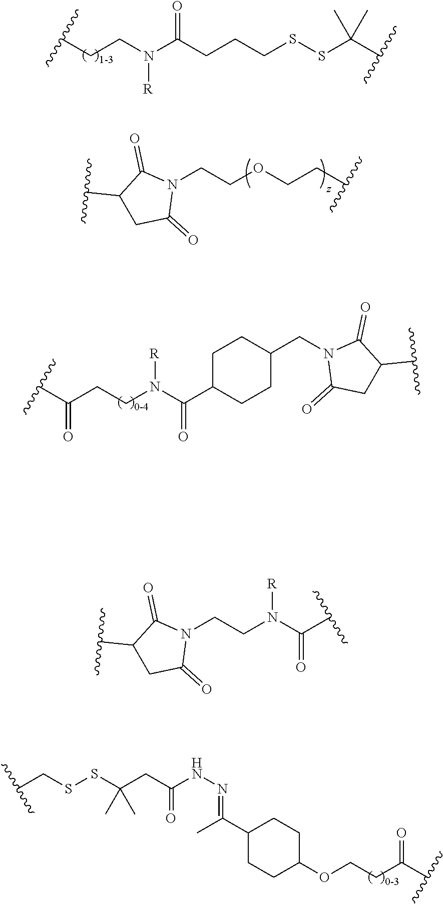

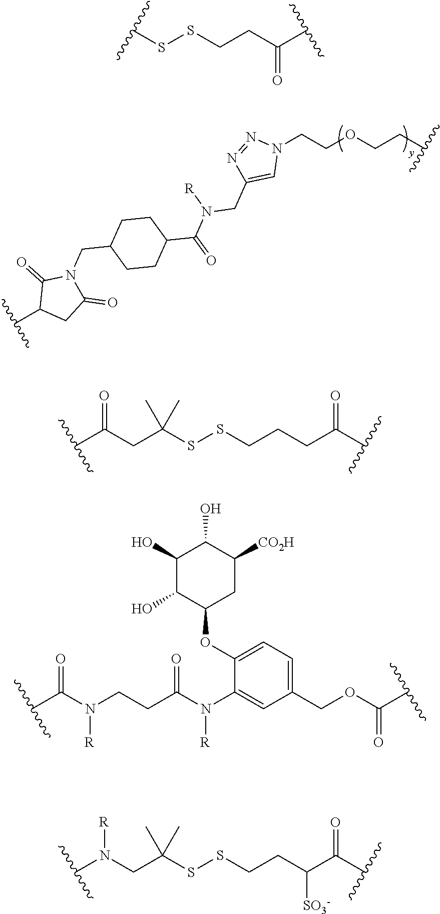

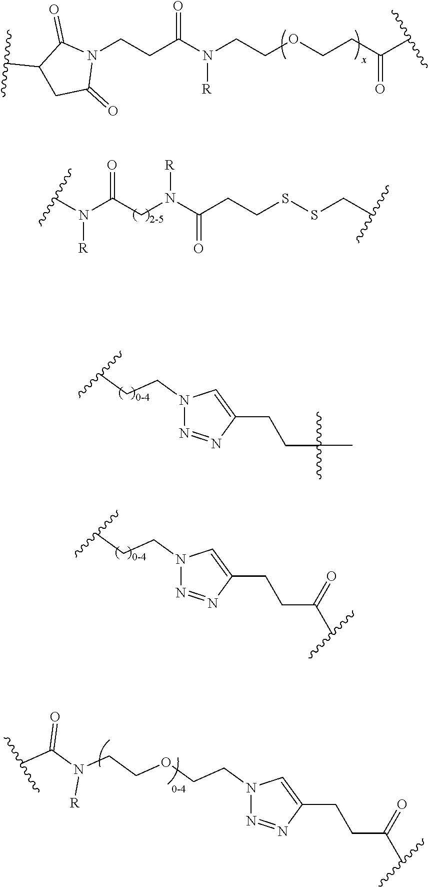

wherein each of k, m, n, p, q, r, s, t, u, v, w, x, y, and z is 1-20; and
R is hydrogen or C1-10 aliphatic.

In some embodiments, k is 3.

In some embodiments, m is 3.

In some embodiments, n is 2. In some embodiments, n is 12.

In some embodiments, p is 3.

In some embodiments, each of m and p is 3.

In some embodiments, q is 1.

In some embodiments, r is 3. In some embodiments, r is 4. In some embodiments, r is 6.

In some embodiments, s is 3. In some embodiments, s is 4. In some embodiments, s is 6.

In some embodiments, each of r and s is 3. In some embodiments, each of r and s is 4. In some embodiments, each of r and s is 6.

In some embodiments, t is 3. In some embodiments, t is 5.

In some embodiments, u is 3. In some embodiments, u is 5.

In some embodiments, each of t and u is 3. In some embodiments, each of t and u is 5.

In some embodiments, v is 3.

In some embodiments, w is 4.

In some embodiments, x is 8.

In some embodiments, y is 2.

In some embodiments, z is 1.

### Payload

Those skilled in the art, reading the present disclosure, will appreciate that any of a variety of payloads can usefully be conjugated with sortilin binding moieties as described herein. Those skilled in the art, reading the present disclosure, will further appreciate that its teachings are not limited to particular specific payloads.

In many embodiments, a useful payload moiety may achieve (e.g., may correlate with) a particular biological or physiological impact on a sortilin or sortilin-containing system, such as a sortilin expressing cell or tissue). In some embodiments, a useful payload may impart detectability to a conjugate (and/or to a sortilin or sortilin-containing system, such as a sortilin expressing cell or tissue). Those skilled in the art are familiar with categories of therapeutic agents based on their biological impacts (see, for example, www.fda.gov/regulatory-information/fdaaa-implementation-chart/usp-therapeutic-categories-model-guidelines, incorporated herein by reference).

In many embodiments, an effect of a payload moiety is a change in one or more parameters of one or more target(s) of interest (e.g., an expression parameter and/or activity of the target of interest). In some embodiments, a target of interest may be a particular gene or gene product, or form (e.g., disease-associated form, splice variant form, etc) thereof.

In some embodiments, a payload may be or comprise a cytotoxic or cytostatic agent, particularly where provided sortilin binding agents are utilized to treat cancer. In some embodiments, a cytotoxic or cytostatic agent may be or comprise a toxin (e.g., a bacterial toxin such as Diphtheria toxin, Pseudomonas aeruginosa exotoxin A, cholera toxin, etc). In some embodiments, a cytotoxic or cytostatic agent may be or comprise a Maytansinoid, Calicheamicin, Amatoxin, Amanitin or a combination thereof. In some embodiments, a cytotoxic or cytostatic agent may be or comprise an alkaloid, an alkylating agent, an antimetabolite (e.g., Hydroxyurea), an anthracycline, a cytotoxic antibiotic (e.g., Actinomycin D, Doxorubicin, Daunorubicin, Epirubicin, Bleomcins Mitomycin C), an antimetabolite agent, an auristatin, camptothecin, an enzyme (e.g., L-Asparaginase), a folate derivative, a metal complex, a microtubule damaging agent (e.g., Vincristine, Vinblastine, Vinorelbine, Cabazitaxel, Paclitaxel and Docetaxel), a nucleoside analog, a taxane, a topoisomerase-2 inhibitor (e.g., Etoposide), a topoisomerase-1 Inhibitor (e.g., Topotecan, Irinotecan), a vinca alkaloid analog, or a combination thereof.

In some embodiments, a payload may be or comprise an anti-cancer agent. In some embodiments, a payload is selected from the group consisting of alkylating agents, anti-metabolites, anti-tumor antibiotics, boron neutron capture therapy agents, cell cycle inhibitors, kinesin spindle protein inhibitors, microtubule-binding agents, topoisomerase inhibitors, and combinations thereof. In some embodiments, a payload may be or comprises an anticancer-peptide, including D-peptide A, B, C, D. Gomesin, Hepcidin and PTP7. In some embodiments, a payload may be or comprises a targeted drug. In some embodiments, a targeted drug may be or comprise angiogenesis inhibitors (e.g., Bevacizunab, Thalidomide, Endostatin, Angiostatin, Angiopoietin, cannabinoids), EGF receptor inhibitors (e.g., Gefitinib and Erlotinib), monoclonal antibodies (e.g., Rituximab and Trastuzumab), proteasome Inhibitors (e.g., Bortezomib and Thlidomide), tyrosine protein kinase inhibitors (e.g., Imatinib and Dasatinib), or a combination thereof.

In some embodiments, a payload may be or comprise an immunotherapeutic agent. In some embodiments, a payload is selected from the group consisting of checkpoint inhibitors, CAR-T cell therapy, antibodies, oncolytic viruses, or a combination thereof.

In some embodiments, a payload may be or comprise an anti-inflammatory agent (e.g., a phytochemical, a non-steroidal anti-inflammatory drug (NSAID), a steroidal anti-inflammatory drug, an antileukotrine agent, a biologic agent or an immune-selective anti-inflammatory derivative (ImSAID)).

In some embodiments, a payload may be or comprise a phytochemical. In some embodiments, a payload is selected from the group consisting of alkaloids (e.g., Chlorogenic acid, Theobromine, Theophylline), anthocyanins/anthocyanidines (e.g., Cyanidin, Malvidin), carotenoids (e.g., beta-carotene, Lutein, Lycopene), caspaisin, catechins, coumestans, flavan-3-Ols, flavonoids (e.g., Epicatechin, Hesperidin, Isorhamnetin, Kaempferol, Myricetin, Naringin, Nobiletin, Proanthrocyanidins, Quercetin, Rutin, Tangeretin), hydroxycinnamic acids (e.g., chicoric acids, coumarin, ferulic acid, scopoletin), isoflavozone (e.g., Daidzein, Genistein), lignans (e.g., Silymarin) monoterpenes (e.g., Geraniol, Limonene), Omega-3, organosulfides (e.g., Allicin, Glutathione, Indole-3-carbinol, isothiocyanates, Sulforaphane), phenolic acids, phytosterols, resveratrol, saponins, stylbenes, triterpenoids, xanthophylls, monophenols, tubulysins, or a combination thereof.

In some embodiments, a payload moiety is or comprises a nucleic acid. In some embodiments, a payload moiety is or comprises a single-stranded nucleic acid. In some embodiments, a payload moiety is or comprises a double-stranded nucleic acid. In some embodiments, a payload moiety is or comprises an oligonucleotide.

In some embodiments, a nucleic acid has a length within a range of about 10-50 nucleotides, about 10-49 nucleotides, about 10-48 nucleotides, about 10-47 nucleotides, about 10-46 nucleotides, about 10-45 nucleotides, about 10-44 nucleotides, about 10-43 nucleotides, about 10-42 nucleotides, about 10-41 nucleotides, about 10-40 nucleotides, about 10-39 nucleotides, about 10-38 nucleotides, about 10-37 nucleotides, about 10-36 nucleotides, about 10-35 nucleotides, about 10-34 nucleotides, about 10-33 nucleotides, about 10-32 nucleotides, about 10-31 nucleotides, about 10-30 nucleotides, about 10-29 nucleotides, about 10-28 nucleotides, about 10-27 nucleotides, about 10-26 nucleotides, about 10-25 nucleotides, about 10-24 nucleotides, about 10-23 nucleotides, about 10-22 nucleotides, about 10-21 nucleotides, about 10-20 nucleotides, about 10-19 nucleotides, about 10-18 nucleotides, about 10-17 nucleotides, about 10-16 nucleotides, about 10-15 nucleotides, about 10-14 nucleotides, about 10-13 nucleotides, about 10-12 nucleotides, about 10-11 nucleotides. In some embodiments, a nucleic acid has a length within a range of about 11-50 nucleotides, about 12-50 nucleotides, about 13-50 nucleotides, about 14-50 nucleotides, about 15-50 nucleotides, about 16-50 nucleotides, about 17-50 nucleotides, about 18-50 nucleotides, about 19-50 nucleotides, about 20-50 nucleotides, about 21-50 nucleotides, about 22-50 nucleotides, about 23-50 nucleotides, about 24-50 nucleotides, about 25-50 nucleotides, about 26-50 nucleotides, about 27-50 nucleotides, about 28-50 nucleotides, about 29-50 nucleotides, about 30-50 nucleotides, about 31-50 nucleotides, about 32-50 nucleotides, about 33-50 nucleotides, about 34-50 nucleotides, about 35-50 nucleotides, about 36-50 nucleotides, about 37-50 nucleotides, about 38-50 nucleotides, about 39-50 nucleotides, about 40-50 nucleotides, about 41-50 nucleotides, about 42-50 nucleotides, about 43-50 nucleotides, about 44-50 nucleotides, about 45-50 nucleotides, about 46-50 nucleotides, about 47-50 nucleotides, about 48-50 nucleotides, about 49-50 nucleotides.

In some embodiments, a nucleic acid is about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides in length.

In some embodiments, particularly when a polypeptide-encoding nucleic acid is utilized as a payload, a nucleic acid has a length within a range of about 10-20,000 nucleotides, about 10-19,000 nucleotides, about 10-18,000 nucleotides, about 10-17,000 nucleotides, about 10-16,000 nucleotides, about 10-15,000 nucleotides, about 10-14,000 nucleotides, about 10-13,000 nucleotides, about 10-12,000 nucleotides, about 10-11,000 nucleotides, about 10-10,000 nucleotides, about 10-9,500 nucleotides, about 10-9,000 nucleotides, about 10-8,500 nucleotides, about 10-8,000 nucleotides, about 10-7,500 nucleotides, about 10-7,000 nucleotides, about 10-6,500 nucleotides, about 10-6,000 nucleotides, about 10-5,500 nucleotides, about 10-5,000 nucleotides, about 10-4,500 nucleotides, about 10-4,000 nucleotides, about 10-3,500 nucleotides, about 10-3,000 nucleotides, about 10-2,500 nucleotides, about 10-2,000 nucleotides, about 10-1,500 nucleotides, about 10-1,000 nucleotides, about 10-950 nucleotides, about 10-900 nucleotides, about 10-850 nucleotides, about 10-800 nucleotides, about 10-750 nucleotides, about 10-700 nucleotides, about 10-650 nucleotides, about 10-600 nucleotides, about 10-550 nucleotides, about 10-500 nucleotides, about 10-450 nucleotides, about 10-400 nucleotides, about 10-350 nucleotides, about 10-300 nucleotides, about 10-250 nucleotides, about 10-200 nucleotides, about 10-150 nucleotides, about 10-100 nucleotides, about 10-95 nucleotides, about 10-90 nucleotides, about 10-85 nucleotides, about 10-80 nucleotides, about 10-75 nucleotides, about 10-70 nucleotides, about 10-65 nucleotides, about 10-60 nucleotides, about 10-50 nucleotides. In some embodiments, a nucleic acid has a length within a range of about 100-20,000 nucleotides, about 200-20,000 nucleotides, about 300-20,000 nucleotides, about 400-20,000 nucleotides, about 500-20,000 nucleotides, about 600-20,000 nucleotides, about 700-20,000 nucleotides, about 800-20,000 nucleotides, about 900-20,000 nucleotides, about 1,000-20,000 nucleotides, about 1,100-20,000 nucleotides, about 1,200-20,000 nucleotides, about 1,300-20,000 nucleotides, about 1,400-20,000 nucleotides, about 1,500-20,000 nucleotides, about 1,600-20,000 nucleotides, about 1,700-20,000 nucleotides, about 1,800-20,000 nucleotides, about 1,900-20,000 nucleotides, about 2,000-20,000 nucleotides, about 3,000-20,000 nucleotides, about 4,000-20,000 nucleotides, about 5,000-20,000 nucleotides, about 6,000-20,000 nucleotides, about 7,000-20,000 nucleotides, about 8,000-20,000 nucleotides, about 9,000-20,000 nucleotides, about 10,000-20,000 nucleotides, about 11,000-20,000 nucleotides, about 12,000-20,000 nucleotides, about 13,000-20,000 nucleotides, about 14,000-20,000 nucleotides, about 15,000-20,000 nucleotides, about 16,000-20,000 nucleotides, about 17,000-20,000 nucleotides, about 18,000-20,000 nucleotides, about 19,000-20,000 nucleotides.

In some embodiments, a nucleic acid agent, e.g., an oligonucleotide agent or a polypeptide-encoding agent, for use in accordance with the present disclosure may comprise a single strand. In some embodiments, a nucleic acid may comprise more than one strand. In some embodiments, a nucleic acid may comprise one or more double-stranded portions. In some such embodiments, some or all of such portion(s) may be formed by self-hybridization of sequences on a single strand; in some embodiments some or all of such portion(s) may be formed by hybridization of separate strands. In some embodiments, a nucleic acid that includes one or more double-stranded portions may include one or more nicks or gaps and/or one or more bulges or loops.

In some embodiments, a nucleic acid agent, e.g., an oligonucleotide agent, for use in accordance with the present disclosure may include one or more structural features or characteristics relevant to its mode of action. For example, those skilled in the art are aware of extensive literature regarding structural features of, for example, oligonucleotides that trigger degradation of their targets (e.g., by recruiting RNase H (such oligonucleotides often being referred to as “antisense” agents or “ASOs”) and/or Dicer and/or other elements of the RNA-Induced Silencing Complex (RISC) (such oligonucleotides often being referred to as “siRNA” agents) and/or that modulate splicing of target transcripts (e.g., to favor production of one splice form over another) and/or that act as guide RNAs to recruit other machinery (e.g., nucleases such as CRISPR/Cas or dsRNA binding proteins, or conjugates thereof etc) to particular nucleic acid sequences, or as aptamers that bind to particular targets, etc.

In some embodiments, a nucleic acid agent is directed to (e.g., hybridizes with) a target nucleic acid (e.g., a DNA or, more commonly, an RNA, such as an mRNA, that may be present, e.g., expressed in a cell to which an administered agent is delivered). In some embodiments, delivery of a nucleic acid agent inhibits (e.g., reduces level and/or activity of) a target nucleic acid. In some embodiments, delivery of a nucleic acid alters (e.g., cleaves, edits, alters splicing of, etc) a target nucleic acid. In some embodiments, delivery of a nucleic acid agent enhances or protects (e.g., reduces degradation of, activates expression of) a target nucleic acid.

In some embodiments, a target nucleic acid is one whose activity or level (e.g., of a particular form or variant thereof) may be associated with a particular disease, disorder or condition; in some such embodiments, delivery of a nucleic acid agent modulates such activity or level toward a non-diseased state (e.g., activity or level). In some embodiments, a target nucleic acid is one whose activity or level (e.g., of a particular form or variant thereof) is associated with cell viability or lack thereof; in some such embodiments, delivery of a nucleic acid agent reduces cell viability (e.g., induces cell death or apoptosis, etc).

In some embodiments, a target is a specific allele with respect to which expression and/or activity of one or more products (e.g., RNA and/or protein products) are intended to be altered. In many embodiments, a target allele is one whose presence and/or expression is associated (e.g., correlated) with presence, incidence, and/or severity, of one or more diseases and/or conditions. Alternatively or additionally, in some embodiments, a target allele is one for which alteration of level and/or activity of one or more gene products correlates with improvement (e.g., delay of onset, reduction of severity, responsiveness to other therapy, etc) in one or more aspects of a disease and/or condition.

In some embodiments, where presence and/or activity of a particular allele (a disease-associated allele) is associated (e.g., correlated) with presence, incidence and/or severity of one or more disorders, diseases and/or conditions, a different allele of the same gene exists and is not so associated, or is associated to a lesser extent (e.g., shows less significant, or statistically insignificant correlation). In some such embodiments, oligonucleotides and methods thereof as described herein may preferentially or specifically target the associated allele relative to the one or more less-associated/unassociated allele(s), thus mediating allele-specific suppression.

In some embodiments, a target sequence is a sequence to which an oligonucleotide agent as described herein binds. In many embodiments, a target sequence is identical to, or is an exact complement of, a sequence of a provided oligonucleotide, or of consecutive residues therein (e.g., a provided oligonucleotide includes a target-binding sequence that is identical to, or an exact complement of, a target sequence). In some embodiments, a target-binding sequence is an exact complement of a target sequence of a transcript (e.g., pre-mRNA, mRNA, etc.). A target-binding sequence/target sequence can be of various lengths to provide oligonucleotides with desired activities and/or properties. In some embodiments, a target-binding sequence/target sequence comprises 5-50 (e.g., 10-40, 15-30, 15-25, 16-25, 17-25, 18-25, 19-25, 20-25, 10, 11, 12, 13, 14, 15, 16, 17, 18, 19, 20, 21, 22, 23, 24, 25, or more) bases. In some embodiments, a small number of differences/mismatches is tolerated between (a relevant portion of) an oligonucleotide and its target sequence, including but not limited to the 5′ and/or 3′-end regions of the target and/or oligonucleotide sequence. In many embodiments, a target sequence is present within a target gene. In some embodiments, a target sequence is present within a transcript (e.g., an mRNA and/or a pre-mRNA) produced from a target gene. In some embodiments, a target sequence includes one or more allelic sites (i.e., positions within a target gene at which allelic variation occurs). In some embodiments, an allelic site is a mutation. In some embodiments, an allelic site is a SNP, wherein SNP refers to single-nucleotide polymorphism (a single nucleotide is substituted compared with original sequence). In some such embodiments, an oligonucleotide agent binds to one allele preferentially or specifically relative to one or more other alleles. In some embodiments, an oligonucleotide agent binds preferentially to a disease-associated allele. For example, in some embodiments, an oligonucleotide agent (or a target-binding sequence portion thereof) has a sequence that is identical to, or an exact complement of a particular allelic version of a target sequence. In some embodiments, a target sequence is a sequence of a particular allele. In some embodiments, an oligonucleotide agent (or a target-binding sequence portion thereof) has a sequence that is identical to, or an exact complement of a target sequence comprising an allelic site, or an allelic site, of a disease-associated allele. In some embodiments, an oligonucleotide agent has a target binding sequence that is an exact complement of a target sequence comprising an allelic site of a transcript of an allele (in many embodiments, a disease-associated allele), wherein the allelic site is a mutation. In some embodiments, an oligonucleotide agent has a target binding sequence that is an exact complement of a target sequence comprising an allelic site of a transcript of an allele (in many embodiments, a disease-associated allele), wherein the allelic site is a SNP.

In some embodiments, a nucleic acid is or comprises an interfering RNA (RNAi) agent. In some embodiments, an RNA is or comprises a single-stranded interfering RNA (ssRNAi) agent. In some embodiments, an RNA is or comprises a double-stranded interfering RNA (dsRNAi) agent. In some embodiments, an RNA is or comprises a short interfering RNA (siRNA) agent. In some embodiments, an RNA is or comprises a micro RNA (miRNA) agent. In some embodiments, a nucleic acid is or comprises a guide RNA (gRNA) agent.

In some embodiments, a nucleic acid is or comprises a short interfering RNA (siRNA) agent. In some embodiments, a nucleic acid comprising an siRNA agent can be linked to a sortilin binding moiety (e.g., directly or indirectly) at a sense strand. In some embodiments, a nucleic acid comprising an siRNA agent can be linked to a sortilin binding moiety (e.g., directly or indirectly) at an antisense strand. In some embodiments, a nucleic acid comprising an siRNA agent can be linked to a sortilin binding moiety (e.g., directly or indirectly) at a 5′ end of an siRNA agent. In some embodiments, a nucleic acid comprising an siRNA agent can be linked to a sortilin binding moiety (e.g., directly or indirectly) at a 3′ end of an siRNA agent.

In some embodiments, a nucleic acid is or comprises an exon skipping agent, an exon inclusion agent, or other splicing modulator.

In some embodiments, a nucleic acid is or comprises an aptamer agent.

In some embodiments, a nucleic acid agent is or comprises an antisense oligo (ASO). In some embodiments, an ASO modulates gene expression via RNase H mediated mechanisms. In some embodiments, an ASO modulates gene expression via steric hindrance.

In some embodiments, a nucleic acid agent is or comprises a phosphorodiamidate morpholino oligonucleotide (PMO).

In some embodiments, a nucleic acid agent is or comprises a peptide-nucleic acid (PNA).

In some embodiments, a nucleic acid agent is or comprises a nucleic acid analog, e.g., an RNA analog or a DNA analog, or a combination thereof.

In some embodiments, a nucleic acid agent is or comprises a sense or antisense strand (or both) of a polypeptide-coding sequence. In some embodiments, delivery of such a nucleic acid results in expression of such encoded polypeptide. In some embodiments, an encoded polypeptide has an activity that improves a cell state (e.g., tends to move it away from a state characteristic of a disease, disorder or condition). In some embodiments, an encoded polypeptide corrects a defect (e.g., replaces a missing activity) in a cell. In some embodiments, an encoded polypeptide is toxic to a cell or otherwise decreases cell viability and/or proliferation. In some embodiments, an encoded polypeptide is or comprises antigen binding sequences of an immunoglobulin (e.g., of an antibody agent such as an scFv, a camelid antibody, a heavy or light chain of an antibody, etc). In some embodiments, an encoded polypeptide suppresses an undesirable biological event or response (e.g., that is associated with a disease and/or with an undesirable immune response, etc). In some embodiments, an encoded polypeptide supports or activates a desirable biological event or response (e.g., immune response).

In some embodiments, a nucleic acid can be linked to a sortilin binding moiety (e.g., directly or indirectly) at a sense strand. In some embodiments, a nucleic acid can be linked to a sortilin binding moiety (e.g., directly or indirectly) at an antisense strand. In some embodiments, a nucleic acid can be linked to a sortilin binding moiety (e.g., directly or indirectly) at a 5′ end of a nucleic acid. In some embodiments, a nucleic acid can be linked to a sortilin binding moiety (e.g., directly or indirectly) at a 3′ end of a nucleic acid.

For example, in some embodiments, a nucleic acid analog includes one or more modified (relative to canonical DNA and/or RNA) nucleotides. In some embodiments, a modified nucleotide comprises one or more of: a modified backbone, a modified nucleobase, a modified sugar (e.g., a modified ribose, or a modified deoxyribose), or a combination thereof. In some embodiments, a modified nucleotide may be or comprise one or more naturally occurring modifications; in some embodiments a modified nucleotide may be or comprise one or more non-naturally-occurring modifications.

In some embodiments, a nucleic acid analog comprises one or more linkages that is not a phosphodiester linkage (e.g., that is or comprises a phosphorothioate linkage or a phosphorodiamidate linkage).

In some embodiments, a nucleic acid agent has a negative charge.

In some embodiments, a nucleic acid agent is substantially uncharged, e.g., has a neutral charge.

Those skilled in the art, reading the present disclosure, will appreciate that, in some embodiments, a nucleic acid agent for use in accordance with the present disclosure may include one or more DNA residues or analogs thereof, one or more RNA residues or analogs thereof, and/or combinations thereof. Furthermore, such skilled person will appreciate that, in some embodiments, a nucleic acid agent may include one or more, or entirely, phosphodiester linkages, phosphorothioate linkages, or other suitable linkages.

In some embodiments, a nucleic acid agent comprises natural residues, e.g., DNA residues and/or RNA residues.

In some embodiments a nucleic acid agent comprises one or more analogs, e.g., DNA analogs and/or RNA analogs.

In some embodiments, a nucleic acid agent comprises DNA residues and/or RNA residues, e.g., natural residues or analogs.

In some embodiments, a nucleic acid comprises one or more chiral centers (e.g., as may be present in, for example, a phosphorothioate linkage). In some embodiments, a preparation of a nucleic acid having a chiral center is stereopure with respect to that center in that it includes only one stereoisomer of that center. In some embodiments, both stereoisomers are present. In some embodiments, the preparation represents a racemic mixture of stereoisomers at that position. In some embodiments, a preparation of a nucleic acid having more than one chiral linkage may be stereopure with respect to one or more centers and mixed (e.g., racemic) with respect to one or more others. In some embodiments, a preparation may be stereopure at all chiral centers. In some embodiments, a preparation may be racemic (e.g., at all chiral centers or overall).

In some embodiments, a nucleic acid comprises one or more modified nucleotides. In some embodiments, a modified nucleotide comprises one or more of: a modified backbone, a modified nucleobase, a modified ribose, a modified deoxyribose, or a combination thereof.

In some embodiments, a modified nucleotide is chosen from: a 2′-O-methyl modified nucleotide, a 5-methylcytidine, a 5-methyluridine, a nucleotide comprising a 5′-phosphorothioate group, a morpholino nucleotide (e.g., a PMO), or a terminal nucleotide linked to a cholesteryl derivative or a dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide (e.g., PMO), a phosphoramidate, a phosphoryl guanidine (PN) based backbone, or a non-natural base comprising nucleotide, or a combination thereof.

In some embodiments, a modified nucleobase comprises a C7-modified deaza-adenine, a C7-modified deaza-guanosine, a C5-modified cytosine, a C5-modified uridine, N1-methyl-pseudouridine (m1ψ), 1-ethyl-pseudouridine (e1ψ), 5-methoxy-uridine (mo5U), 5-methyl-cytidine (m5C), pseudouridine (ψ), 5-methoxymethyl uridine, 5-methylthio uridine, 1-methoxymethyl pseudouridine, 5-methyl cytidine, 5-methoxy cytidine, or a combination thereof.

In some embodiments, a modified sugar (e.g., a modified ribose, or a modified deoxyribose) comprises: a 2′fluoro modification, a 2′-O-methyl (2′OMe) modification, a locked nucleic acid (LNA), a 2′-fluoro arabinose nucleic acid (FANA), a hexitol nucleic acid (HNA), a 2′O-methoxyethyl (2′MOE) modification, or a combination thereof.

In some embodiments, a modified backbone comprises a phosphorothioate (PS) modification, a phosphoryl guanidine (PN) modification, a borano-phosphate modification, an alkyl phosphonate nucleic acid (phNA), a peptide nucleic acid (PNA), or a combination thereof.

In some embodiments, a nucleic acid comprises one or more modifications, e.g., to a 5′ end of an oligonucleotide. In some embodiments, a nucleic acid comprises a 5′ amino modification.

In some embodiments, a nucleic acid is partially modified (e.g., at least 5%) for a particular modification, e.g., throughout the length of a sequence.

In some embodiments, a nucleic acid is fully modified for a particular modification throughout the length of a sequence.

In some embodiments, at least 5% of a particular nucleotide (e.g., A, G, C, T, or U) is modified in an oligonucleotide.

In some embodiments, all (e.g., 100%) of a particular nucleotide (e.g., A, G, C, T, or U) is modified in an oligonucleotide.

In some embodiments, a nucleic acid agent comprises a structure comprising a first wing sequence, a gap sequence, and a second wing sequence. A nucleic acid comprising such a wing-gap-wing sequence is typically referred to as a gapmer. In some embodiments, a gap sequence is flanked by a first wing sequence and a second wing sequence. In some embodiments, a gap sequence comprises about 6-10 nucleotides. In some embodiments, a wing sequence comprises one or more nucleotides. In some embodiments, a wing sequence comprises one or more modified nucleotides, e.g., as disclosed herein. In some embodiments, a gapmer acts by recruiting RNaseH.

In some embodiments, a nucleic acid comprises an overhang. In some embodiments, an overhang is a 3′ overhang or a 5′ overhang. In some embodiments, an overhang is a 3′ overhang. In some embodiments, an overhang comprises at least 1, 2, 3, 4, 5, 6, 7, 8, 9, or 10 nucleotides. In some embodiments, a nucleic acid is double-stranded and comprises an overhang.

In some embodiments, a nucleic acid, e.g., an oligonucleotide, is characterized in that when delivered to a cell, tissue, or organism expressing a target, expression and/or activity of a target is modulated, e.g., reduced, as compared to a cell, tissue, or organism, which has not been delivered an oligonucleotide.

Without wishing to be bound by theory, it is believed that in some embodiments, a sortilin binding moiety, e.g., a peptide as disclosed herein, can be conjugated to a payload moiety comprising a nucleic acid, e.g., an oligonucleotide.

In some embodiments, a payload may be or comprise particles (e.g., microparticles or nanoparticles) such as, for example, metal particles, crystalline particles, polymer particles, lipid-containing particles, etc. Those skilled in the art will be familiar with a variety of particulate systems that are useful in the delivery of payload agents. For example, a variety of polymeric (e.g., polyacrylamide, polyacrylate, or polysaccharide-containing, specifically including PLGA-based, systems have been described. Inorganic particle (e.g., nanoparticle) systems have also been reported, such as that utilize metals (e.g., gold, titanium) for detection and/or association with actives. In some embodiments, payloads may be or comprise quantum dots.

A variety of lipid-based and/or viral-based particle systems have been described as useful for delivery of nucleic acids and may be utilized in or as payloads in accordance with the present disclosure. In some embodiments, liposomes or lipid nanoparticles are utilized.

### Preparations and Compositions

The present disclosure, among other things, provides particular preparations of sortilin binding agents (e.g., sortilin binding moieties or conjugate agents).

For example, in some embodiments, the present disclosure provides liquid preparations. In some embodiments, the present disclosure provides solid preparations (e.g., powder preparations, such as lyophilized compositions, nebulization preparations, or tablet or capsule preparations, etc.).

In some embodiments, provided compositions are pharmaceutical compositions that comprise or otherwise deliver a sortilin binding agent (e.g., a sortilin binding moiety or a conjugate agent); typically, such pharmaceutical compositions comprise an active agent (e.g., a sortilin biding agent which may, in some embodiments, be or comprise a sortilin binding moiety, or may be or comprise a conjugate agent, or a composition comprising the same) and one or more pharmaceutically or physiologically acceptable carriers, diluents, or excipients.

In some embodiments, a pharmaceutical composition described herein may comprise a buffer such as neutral buffered saline or phosphate buffered saline (PBS); a carbohydrate, such as glucose, mannose, sucrose, dextrans, or mannitol; a protein, polypeptide, or amino acid (e.g., glycine); an antioxidant; a chelating agent, such as EDTA or glutathione; an adjuvant (e.g., aluminum hydroxide); and/or a preservative. In some embodiments, a pharmaceutical composition is substantially free of one or more specified contaminants; for example, in some embodiments, a particular contaminant, or set of contaminants, is not present in the composition above a specified threshold level. In some such embodiments, a relevant contaminant may be or comprise an endotoxin.

In some embodiments, pharmaceutical compositions described herein may be administered in a manner appropriate to a disease, disorder, or condition to be treated or prevented. In some embodiments, quantity and/or frequency of administration may be determined by such factors as condition of a patient, and/or type and/or severity of a patient's disease, disorder, or condition, although appropriate dosages may be determined by clinical trials.

In some embodiments, a pharmaceutical composition provided by the present disclosure may be in a form such as, for example, liquid, semi-solid and solid dosage forms, such as liquid solutions (e.g., injectable and infusible solutions), dispersions or suspensions, liposomes, and suppositories. Typically, pharmaceutical compositions that comprise or deliver antibody agents are injectable or infusible solutions; in some such embodiments, such compositions can be formulated for administration intravenously, subcutaneously, intradermally, intranasally, intratumorally, intramedullary, intramuscularly, intranodally, intraperitoneally, intrathecally, sublingually, topically or transarterially. In some embodiments, provided pharmaceutical compositions are formulated for intravenous administration. In some embodiments, provided pharmaceutical compositions are formulated for subcutaneous administration.

Pharmaceutical compositions described herein can be formulated for administration by using infusion techniques that are commonly known in the field (See, e.g., Rosenberg et al., New Eng. J. of Med. 319:1676, 1988, which is hereby incorporated by reference in its entirety).

In some embodiments, pharmaceutical compositions described herein are administered in combination with (e.g., before, simultaneously, or following) another therapy for a particular disease, disorder, or condition, or symptom thereof e.g., a standard-of-care (“SOC”) therapy for such disease, disorder or condition, or symptom thereof. In some embodiments, pharmaceutical compositions described herein may be administered before or following surgery.

In some embodiments, a provided sortilin binding agent is useful in treatment of cancer and may be administered in combination with other cancer therapy such as, for example, one or more of surgery, radiation, immunotherapy, checkpoint inhibitor therapy, etc.

### Production

### Sortilin-Binding Peptides

Those skilled in the art, reading the present disclosure, will appreciate that various technologies are available that can be utilized to prepare a of a sortilin binding moiety. In some embodiments, a sortilin binding moiety is prepared according to methods described in this disclosure. In some embodiments, a sortilin binding moiety is prepared by solid phase peptide synthesis protocols. Many reviews of solid phase peptide synthesis exist for example Coin et al, Nat. Protoc. 2007, 2, 3247-3256.

In some embodiments, one or more peptides provided and/or utilized in accordance with the present disclosure may be cyclic peptides. Those skilled in the art will be aware of a variety of technologies for achieving peptide cyclization. In some embodiments, cyclization may involve only side chain residues; in some embodiments, cyclization may involve only backbone residues; in some embodiments, cyclization may involve both side chain and backbone residues.

In some embodiments, cyclization may involve disulfide bond formation, amide bond formation, ester bond formation, stapling, etc.

The present disclosure exemplifies certain cyclization strategies can be utilized to form a provided sortilin binding moiety comprising a cyclic peptide. In some embodiments, a provided sortilin binding agent or moiety comprises at least two cysteine residues which form a disulfide bond. Those skilled in the art, reading the present disclosure, will appreciate various alternative cyclization strategies are available. In some embodiments, the two cysteines forming the intramolecular disulfide are exchanged with canonical and non-canonical amino acids that are capable of forming covalent bonds, including amides, disulphides, thioethers, diselenides, triazoles, azo-bridge. In some embodiments, a cyclic sortilin binding moiety is formed by conjugation between two cysteine residues within the peptide sequence and a molecular scaffold comprising thiol-reactive groups (e.g.

hexafluorobenzene or di(bromomethyl)benzene). In some embodiments, a bicyclic sortilin binding moiety is formed by conjugation between three cysteine residues within the peptide sequence and a molecular scaffold comprising thiol-reactive groups (e.g. 1,3,5-Tri(bromomethyl)benzene). In some embodiments, a molecular scaffold comprising thiol-reactive groups is selected from

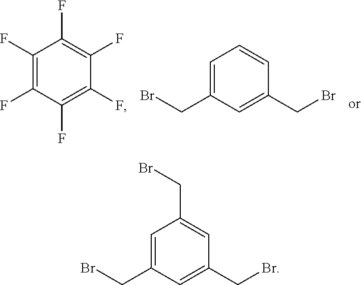

In some embodiments, a cyclic sortilin binding moiety is formed by conjugation between an acidic side chain of certain amino acids (e.g. aspartic acid, glutamic acid) and basic side chains of certain amino acids (e.g. lysine) within the peptide sequence. In some embodiments, the acidic and basic side chains of certain amino acids within the peptide sequence can form an amide bond.

In some embodiments, a cyclic sortilin binding moiety is formed by conjugation between the peptide N-terminus and reactive side chains of certain amino acids within the peptide sequence. In some embodiments, such strategy is called “head to side chain” cyclization. In some embodiments, the N-terminus and a reactive side chains of certain amino acids within the peptide sequence (e.g., aspartic acid, glutamic acid) can form an amide bond. In some embodiments, the N-terminus and a reactive side chains of certain amino acids within the peptide sequence can form a thioether bond.

In some embodiments, a cyclic sortilin binding moiety is formed by introducing staples between certain side chains of certain amino acids within the peptide sequence. A staple is a linker that can link one amino acid residue to another amino acid residue through bonding two peptide backbone atoms of the amino acid residues and, as is understood by those skilled in the art, the resulting bond is not through the peptide backbone between the linked amino acid residues. In some embodiments, a staple bonds to the peptide backbone by replacing one or more hydrogen and/or substituents (e.g., side chains, O, S, etc.) on peptide backbone atoms (e.g., C, N, etc.). Those skilled in the art, reading the present disclosure, will appreciate that a variety of peptide stapling technologies are available, including both hydrocarbon-stapling and non-hydrocarbon stapling technologies. In some embodiments, a staple is a hydrocarbon staple. In some embodiments, a staple is a non-hydrocarbon staple. In some embodiments, a non-hydrocarbon staple comprises one or more chain heteroatoms wherein a chain of a staple is the shortest covalent connection within the staple from one end of the staple to the other end of the staple. In some embodiments, a non-hydrocarbon staple is a comprises at least one sulfur atom derived from an amino acid residue of a polypeptide. In some embodiments, a nonhydrocarbon staple comprises two sulfur atom derived from two different amino acid residues of a polypeptide. In some embodiments, a non-hydrocarbon staple comprises two sulfur atoms derived from two different cysteine residues of a polypeptide. In some embodiments, a staple is a cysteine staple. In some embodiments, a staple is a non-cysteine staple. In some embodiments, amino acid residues having side chains comprising double or triple bonds and optionally various heteroatoms may be utilized to construct staples.

### Conjugates

In some embodiments, a conjugate agent is prepared by conjugating or covalently linking a payload moiety to a sortilin binding moiety. In some embodiments, the payload moiety may be linked to a sortilin binding moiety by, for example reaction of the payload moiety in-solution with a sortilin binding moiety.

In some embodiments, conjugates can have two or more payloads. In some embodiments, two identical payload moieties are linked to a sortilin binding moiety. In some embodiments, two different payload moieties are linked to a sortilin binding moiety. In some embodiments, the payload moiety may be linked to two or more sortilin binding moieties. In some embodiments, the payload moiety may be linked to two or more different binding moieties. In some embodiments, the payload moiety may be linked to a two or more binding moieties, wherein one of the binding moieties is a sortilin binding moiety.

In some embodiments, whether cleavable or non-cleavable, a linker can be installed by a chemical linking reaction between the payload and the binding moiety to which it is being conjugated. The payload and binding moiety may or may not be first modified to increase or facilitate reactivity towards one another. Such modification can also increase or improve the specificity of the conjugation reaction and degree of conjugation when that is desired. In some embodiments, linker may be installed in a single reaction or by stepwise reactions until the desired conjugate have been prepared.

Non-limiting examples of chemical linking reactions to form conjugate agents include reaction between carboxylic acids and amines, thiols or alcohols (i.e., nucleophiles) to form amides, reaction of various thiols to form disulfides, reaction between thiols and alkyl halides or maleimides to form thioethers, reaction of alkynes with azides to form triazoles (“Click Reaction”), reaction between aldehydes and hydrazides or amines, or aminoxy compounds to form hydrazones, imines and oxy imines, thioesters and esters. The carboxylic acids may be activated in situ in the presence of the amines, thiols or alcohols so as to be made reactive or may be pre-activated prior to addition of the nucleophile, for example by converting to activated esters of N-hydroxysuccinimide (NHS) or sulfonated-NHS. Many reviews of chemical linking reactions exist for example Spicer et al. (2018) Chem. Rev. 2018, 118, 16, 7702-7743.

### Uses

Those skilled in the art, reading the present disclosure, will appreciate a variety of uses for provided sortilin binding agents (e.g., sortilin binding moieties and/or conjugates that include a sortilin binding moiety and at least a payload moiety). In some embodiments, provided sortilin binding agents are useful to bind sortilin. In some embodiments, provided sortilin binding agents are usefully internalized into sortilin-expressing cells. In some embodiments, provided sortilin binding agents are useful to deliver a payload to (or, in some embodiments, into) sortilin-expressing cells.

In some embodiments, a provided sortilin binding agent is contacted with a system that comprises sortilin, such as for example that is or comprises one or more cells that express sortilin (e.g., on surfaces thereof). In some embodiments, a system is an in vitro system. In some embodiments, a system comprises cultured cells. In some embodiments, a system comprises a cell line. In some embodiments, a system is or comprises an organism.

In some embodiments, a provided sortilin binding agent is administered to a subject (e.g., to a human or animal subject), for example to achieve binding to one or more sortilin-expressing cells or tissues in such subject. In some such embodiments, such binding is detectable (e.g., when a bound agent includes a payload whose presence is detectable, e.g., via imaging (e.g., of a color or of fluorescence or of a structure such as a nanoparticle, etc) or other means (e.g., detection of radioactivity), etc.

In some embodiments, a sortilin-expressing cell is or comprises a cell (e.g., of a tissue) chosen from: immune cells (e.g., bone marrow cells, lymph node cells, thymic cells, peripheral blood mononuclear cells [e.g., myeloid and/or lymphoid cells], erythrocytes, eosinophils, neutrophils, and/or platelets); nervous system cells (e.g., brain tissue, cortex, cerebellum, retinal cells, spinal cord cells, nerve cells, neurons, and/or supporting cells; endothelial cells; muscle (e.g., heart muscle, smooth muscle, and/or skeletal muscle); small intestine cells; colon cells; adipocytes; kidney cells; liver cells; lung cells; splenic cells; stomach cells; esophagus cells; bladder cells; pancreas cells; thyroid cells; salivary gland cells; adrenal gland cells; pituitary gland cells; breast cells; skin cells; ovary cells; uterus cells; placenta cells; prostate cells; or testis cells, or a combination thereof.

In some embodiments, a sortilin-expressing cell is or comprises a cancer cell, e.g., as described herein. In some embodiments, a sortilin-expressing cancer cell is selected from the group consisting of bladder cancer cells, breast cancer cells, colorectal cancer cells, endometrial cancer cells, glioblastoma cells, kidney cancer cells, liver cancer cells, lung cancer cells, ovarian cancer cells, pancreatic cancer cells, prostate cancer cells, skin cancer cells, small intestine cancer cells, thymus cancer cells, stomach cancer cells, thyroid cancer cells, and combinations thereof.

In some embodiments, a cell to which a provided sortilin-binding agent is contacted with a system that comprises both sortilin and a target of a payload moiety. In some such embodiments, a relevant system is or comprises cells that express both sortilin and such target.

In some embodiments, a provided sortilin-binding agent is administered to a subject having a disease, disorder, or condition e.g., as disclosed herein. In some embodiments, a disease, disorder or condition is one associated with cells in which sortilin and/or a target of a payload moiety is present. In some embodiments, a subject to whom a provided agent is administered is first determined to express sortilin (e.g., in one or more disease-associated cells or tissues). In some such embodiments, the same sortilin binding moiety may be used to detect such sortilin (e.g., due to association with a detectable payload moiety) and to deliver therapeutic payload.

Sortilin-binding agents described herein (e.g., that are or comprise sortilin binding polypeptides) are useful in the treatment of a variety of diseases, disorders and conditions.

In some embodiments, a provided sortilin binding agent is administered to a system (e.g., a subject) expressing sortilin and its binding reduces a relevant level (e.g., a cell surface level) and/or activity of sortilin. Alternatively or additionally, in some embodiments, a sortilin-binding agent delivers a payload to a sortilin-expressing system. In some embodiments, a sortilin-binding agent is administered to a system comprising cells that express sortilin on their surfaces, and the sortilin binding agent is internalized into such cells; in some such embodiments, such internalization accomplishes delivery of a payload (e.g., that may have been associated with the internalized sortilin binding agent, such as by being conjugated thereto) into the cells.

In some embodiments, a relevant disease, disorder or condition to which a conjugate disclosed herein is provided is associated with elevated or otherwise aberrant expression (e.g., surface expression) and/or activity of sortilin. In some embodiments, a relevant disease, disorder or condition is not associated with elevated or otherwise aberrant expression and/or activity of sortilin, but nonetheless can be impacted by binding to sortilin and/or by accomplishing payload delivery via such binding.

Sortilin may be a desirable target for treatment of a variety of diseases, disorders and conditions. Sortilin expression and/or activity has been described as being associated with numerous physiological events, states, etc., and its role(s) in human disease have been subject to extensive study and review (see, for example, Mazella Int J Molec Sci 23:11888, 2022; Mitok et al, J Lipid Res 63:8, 2022; Ayodele et al., Curr Neurol Neurosci Rept 21:1, 2021; Ghaemimanesh et al., J Cell Physiol 236:6271, 2021; Al-Yozbaki et al., BBA—Rev on Cancer 1874:188429, 2020; Blondeau et al., Front Pharmacol 9:1561, 2019; Talbot, Front Pharmacol 9:1507, 2019; etc.)

In some embodiments, a relevant disease, disorder or condition is one or more of a neurological disease, disorder or condition, a metabolic disease, disorder or condition, a cardiovascular disease, disorder or condition, a proliferative disorder, a lysosomal storage disease, an inflammatory disease, disorder or condition, or cancer.

In some embodiments, a relevant disease, disorder or condition is one or more of Alzheimer's disease, an inflammatory disease, Parkinson's disease, diabetes mellitus (e.g., type II diabetes mellitus), cardiovascular disease, a-1 antitrypsin deficiency, a lysosomal storage disease, or a cancer. In some embodiments, a relevant disease, disorder or condition is cancer. In some embodiments, a relevant disease, disorder or condition is cancer, wherein the cancer is a cancer of particular types (e.g., a cancer of the blood, a tumor, a non-tumor cancer, a cancer of the brain, a cancer of the lung, a cancer of the bones, etc.). In some embodiments, a relevant disease, disorder or condition is a cancer selected from the group consisting of: acute lymphoblastic leukemia, acute myeloid leukemia, bladder cancer, bone sarcoma, brain cancer, breast cancer, cervical cancer, colorectal cancer, endometrial cancer, gastric cancer, glioblastoma, glioma, head cancer, Hodgkin lymphoma, kidney cancer, liver cancer, lung cancer, melanoma, neuroblastoma, non-Hodgkin lymphoma, ovarian cancer, pancreatic cancer, prostate cancer, skin cancer, small intestine cancer, soft tissue cancer, spleen cancer, thymus cancer, stomach cancer, testis cancer, thyroid cancer, transitional cell bladder cancer, urothelial cancer, Wilm's tumour and combinations thereof.

In some embodiments, the invention provides a method of diagnosing a disease, disorder or condition in a subject suspected of suffering from such disease, disorder or condition, comprising a) contacting a sample of the subject with the conjugate as described herein under conditions that result in binding of the conjugate with a sortilin-expressing cell, and b) determining binding of the conjugate to sortilin-expressing cells. In some embodiments, a relevant disease, disorder or condition is one or more of a neurological disease, disorder or condition, a metabolic disease, disorder or condition, a cardiovascular disease, disorder or condition, a proliferative disorder, a lysosomal storage disease, or cancer.

### Dosing Regimens

Those skilled in the art will be familiar with dosing regimens utilized with approved therapeutics, including specifically approved peptide therapeutics such as Abaloparatide, Afamelanotide, Angiotensin II, Atosiban, Aviptadil, Bremelanotide, Carbetocin, Carfilzomib, Cosyntropin, Degarelix, Dulaglutide, Enfuvirtide, Eptifibatide, Etelcalcetide, Exenatide, Glatiramer, Gramicidin D, Icatibant, Lepirudin, Leuprolide, Linaclotide, Liraglutide, Lixisenatide, Lucinactant, Mifamurtide, Nesiritide, Oxytocin, Pasireotide, Peginesatide, Plecanatide, Pramlintide, Romiplostim, Semaglutide, Sermorelin, Setmelanotide, Taltirelin, Teduglutide, Teriparatide, Tesamorelin, Thymalfasin, Ziconotide, and approved peptide-drug-conjugate therapeutics such as Melflufen and ¹⁷⁷Lu-dotatate.

In many embodiments, provided sortilin binding agents are administered parenterally. In some embodiments, a provided sortilin binding agent may be administered intravenously, intramuscularly, subcutaneously, etc. In some embodiments, a provided sortilin binding agent may be administered by infusion. In some embodiments, a provided sortilin binding agent may be administered by intrathecal injection. In some embodiments, a provided sortilin binding agent may be administered by inhalation. In some embodiments, a provided sortilin binding agent may be administered by nebulization.

In some embodiments, a conjugate agent is administered at a fixed dose, i.e. independent of body weight.

In some embodiments, a conjugate agent is administered based on body weight, e.g., in a mg/kg dosing.

In some embodiments, a conjugate agent is administered at an initial dose. In some embodiments, an initial dose may be followed by one or more subsequent doses. In some embodiments, one or more subsequent dose may be administered daily, weekly, or monthly, or at other intervals in between. In some embodiments, a dosing regimen disclosed herein may be repeated for one or more times.

In some embodiments, provided methods comprise administering to a patient (e.g., a human patient) in need thereof a conjugate agent or a composition that comprises or delivers a conjugate agent in an amount that is comparable (e.g., corresponds to, is equivalent to) to about 0.1 mg/kg, about 0.3 mg/kg, about 1 mg/kg, about 3 mg/kg, about 5 mg/kg, about 10 mg/kg, about 15 mg/kg, or about 30 mg/kg in a mouse. In some such embodiments, the amount of conjugate agent administered to the subject is about 15 mg, about 25 mg, about 50 mg, about 75 mg, about 150 mg, or about 500 mg.

The present specification is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended Embodiments. Certain advantages and objects characteristic of some embodiments of the invention may not necessarily be characteristic of all embodiments of the invention.

### EMBODIMENTS

1. A conjugate comprising:
(a) polypeptide; and
(b) a payload; and
(c) optionally, a linker,
wherein the polypeptide includes a sortilin binding moiety that:
has a length within a range of about 12 to about 20 amino acids and includes a characteristic sequence represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein each X is independently any canonical or non-canonical amino acid.

2. The conjugate of embodiment 1, wherein the sortilin binding moiety:
corresponds to a C-terminal fragment of progranulin, or a variant thereof, and includes not more than about 20 contiguous residues corresponding to contiguous progranulin residues.

3. The conjugate of embodiment 1, wherein the sortilin binding moiety is or comprises a cyclic peptide.

4. The conjugate of embodiment 1, wherein the characteristic sequence is represented by:

(SEQ ID NO: 3)
(R/N) X²⁻³ C X⁰⁻¹ R (Q/E).

5. The conjugate of embodiment 1, wherein the characteristic sequence is represented by:

(SEQ ID NO: 4)
(R/N) X²⁻³ L X⁰⁻¹ R (Q/B43/B50).

6. The conjugate of embodiment 1, wherein the sortilin binding moiety is or comprises a linear peptide and the characteristic sequence is represented by SEQ ID NO: 2.

7. The conjugate of embodiment 5, wherein the sortilin binding moiety is or comprises a linear peptide.

8. The conjugate any one of preceding embodiments, wherein X is an amino acid included in one or more of Tables 2-7.

9. The conjugate of embodiment 1, wherein the characteristic sequence is represented by:

(SEQ ID NO: 5)
(R/N) X²⁻³ C/L X⁰⁻¹ R Q (L/B13/F02) (L/B13/F02).

10. The conjugate of embodiment 1, wherein the characteristic sequence is represented by:

(SEQ ID NO: 6)
(R/N) X²⁻³ C X⁰⁻¹ R Q (L/B13/F02) (L/B13/F02).

11. The conjugate of embodiment 1, wherein the characteristic sequence includes at least a second cysteine residue and the polypeptide includes at least one disulfide bond between cysteine residues.

12. The conjugate of embodiment 1, wherein the characteristic sequence is represented by:

(SEQ ID NO: 7)
(R/N) X10 X11 X12 C R Q.

13. The conjugate of embodiment 1, wherein the characteristic sequence is represented by:

(SEQ ID NO: 8)
X4 X5 X6 X7 X8 (R/N) X10 X11 X12 CR Q;

wherein at least one of X4, X5, and X6 is a cysteine residue.

14. The conjugate of embodiment 13, wherein the polypeptide includes at least one disulfide bond between cysteine residues.

15. The conjugate of embodiment 1, wherein the characteristic sequence is represented by:

(SEQ ID NO: 9)
(R/E) X4 X5 X6 X7 X8 (R/N) X10 X11 X12 CR Q;

wherein at least one of X4, X5, and X6 is a cysteine residue.

16. The conjugate of embodiment 15, wherein the polypeptide includes at least one disulfide bond between cysteine residues.

17. The conjugate of embodiment 1, wherein the characteristic sequence is represented by:

(SEQ ID NO: 10)
(R/E) X4 X5 X6 X7 X8 (R/N) X10 X11 X12 C R Q
(L/B13/F02) (L/B13/F02);

wherein at least one of X4, X5, and X6 is a cysteine residue.

18. The conjugate of embodiment 17, wherein the polypeptide includes at least one disulfide bond between cysteine residues.

19. The conjugate of embodiment 1, wherein the characteristic sequence is represented by:

(SEQ ID NO: 11)
C R X10 X11 C X13 R Q;

wherein the two cysteine residues form a disulfide bond and the polypeptide is a cyclic polypeptide.

20. The conjugate of embodiment 1, wherein the characteristic sequence is represented by:

(SEQ ID NO: 12)
(R/H) N X6 X7 C R X10 X11 C X13 R Q;

wherein the two cysteine residues form a disulfide bond and the polypeptide is a cyclic polypeptide.

21. The conjugate of embodiment 1, wherein the characteristic sequence is represented by:

(SEQ ID NO: 13)
(R/H) N X6 X7 C R X10 X11 C X13 R Q (L/B13/F02)
(L/B13/F02);

wherein the two cysteine residues form a disulfide bond and the polypeptide is a cyclic polypeptide.

22. The conjugate of any one of the preceding embodiments, wherein the sortilin binding moiety includes one or more of:
(i) a basic residue such as L-arginine or an analog thereof at a position corresponding to position P3 and/or P4 of a 17mer C-terminal fragment of progranulin;
(ii) a hydrophobic residue such as a L-tryptophan or an analog thereof at a position corresponding to position P4 of a 17mer C-terminal fragment of progranulin;
(iii) a long hydrophobic residue at a position corresponding to position P10 a 17mer C-terminal fragment of progranulin;
(iv) a covalent bond such as a disulfide bond) between residues at positions corresponding to positions P5 and P13 or P8 and P12 of a 17mer C-terminal fragment of progranulin; and
(v) a hydrophobic residue such as Leucine or a Leucine at a position corresponding to position P16 and/or P17 of a 17mer C-terminal fragment of progranulin.

23. The conjugate of any one of the preceding embodiments, wherein X represents a canonical amino acid.

24. The conjugate of any one of the preceding embodiments, wherein X is a non-canonical amino acid.

25. The conjugate of embodiment 24 wherein the non-canonical amino acid is selected from those in one or more of Tables 2-7.

26. The conjugate of any one of the preceding embodiments, wherein the payload is or comprises a therapeutic payload.

27. The conjugate of embodiment 1, wherein the sortilin binding moiety is characterized in that:
(i) it demonstrates affinity (Kd) for human sortilin 1 below about 1 μM when assessed by fluorescence polarization;
(ii) in a competitive binding assay with a reference C-terminal progranulin fragment, it demonstrates an IC₅₀ less than about 12 μM.

28. The conjugate of embodiment 27, wherein the sortilin binding moiety is further characterized in that, when maintained under murine serum, it displays stability greater than that of the reference C-terminal progranulin fragment.

29. The conjugate of embodiment 27, wherein the sortilin binding moiety is further characterized in that, when maintained under murine serum, it displays stability lower than that of the reference C-terminal progranulin fragment.

30. The conjugate of any one of embodiments 27-29, wherein the reference C-terminal progranulin fragment has an amino acid sequence that is or comprises APRWDAPLRDPALRQLL (SEQ ID NO: 29).

31. The conjugate of any one of embodiments 27-29, wherein the sortilin binding moiety is characterized by a stability half-life in murine serum that is greater than about >3 minutes.

32. The conjugate of embodiment 31, wherein the stability half-life in murine serum is about 5 minutes.

33. The conjugate of embodiment 31, wherein the stability half-life in murine serum is about 10 minutes.

34. The conjugate of embodiment 31, wherein the stability half-life in murine serum is about 14 minutes.

35. The conjugate of embodiment 30, characterized by an IC50 in the competitive binding assay that is less than about 5000 nM.

36. The conjugate of embodiment 35, wherein the IC50 is less than about 4000 nM.

37. The conjugate of embodiment 35, wherein the IC50 is less than about 3000 nM.

38. The conjugate of embodiment 35, wherein the IC50 is less than about 2000 nM.

39. The conjugate of embodiment 35, wherein the IC50 is less than about 1000 nM.

40. The conjugate of embodiment 35, wherein the IC50 is less than about 750 nM.

41. The conjugate of embodiment 35, wherein the IC50 is about 600-650 nM.

42. The conjugate of any one of embodiments 35-41, wherein the sortilin binding moiety is or comprises a cyclic peptide.

43. The conjugate of embodiment 35, wherein the IC50 is less than about 600 nM.

44. The conjugate of embodiment 35, wherein the IC50 is less than about 500 nM.

45. The conjugate of embodiment 35, wherein the IC50 is less than about 400 nM.

46. The conjugate of embodiment 35, wherein the IC50 is less than about 300-400 nM.

47. The conjugate of embodiment 35, wherein the IC50 is less than about 300-350 nM.

48 The conjugate of any one of embodiment 43, wherein the sortilin binding moiety is or comprises a linear peptide.

49. The conjugate of embodiment 1, wherein the conjugate selectively or specifically targets sortilin-expressing cells.

50. The conjugate of embodiment 1, wherein the conjugate selectively or specifically targets cancer cells over normal cells.

51. The conjugate of embodiment 50, wherein the payload is or comprises a cytotoxic moiety and the conjugate kills the sortilin-expressing cells.

52. The conjugate of embodiment 51, wherein the conjugate kills the sortilin expressing cells with a potency greater than that observed for an otherwise identical conjugate whose sortilin binding moiety is or comprises a reference peptide that is a C-terminal fragment of progranulin.

53. The conjugate of embodiment 52, wherein the reference peptide has amino acid sequence APRWDAPLRDPALRQLL (SEQ ID NO: 29).

54. The conjugate of embodiment 53 wherein the potency is at least about 5 fold greater.

55. The conjugate of embodiment 50, wherein the cancer cells express sortilin at a level equivalent or above that at which sortilin is expressed by otherwise comparable noncancerous cells.

56. The conjugate of embodiment 1, wherein the conjugate undergoes cellular internalization.

57. The conjugate of embodiment 1, characterized in that the conjugate demonstrates improved affinity for sortilin relative to that observed with the unconjugated sortilin-binding moiety.

58. The conjugate of embodiment 57, wherein such improved affinity is at least about 2 fold greater.

59. The conjugate of embodiment 1, wherein the payload is or comprises a therapeutic or diagnostic moiety.

60. The conjugate of embodiment 1, wherein the payload is or comprises a therapeutic moiety.

61. The conjugate of embodiment 1, wherein the payload is or comprises a cytostatic or cytotoxic moiety.

62. The conjugate of embodiment 61, wherein the payload is or comprises a cytotoxic moiety.

63. The conjugate of embodiment 62, wherein the cytotoxic moiety is characterized by a subnanomolar IC50 with respect to relevant cells.

64. The conjugate of embodiment 61, wherein the cytotoxic moiety is or comprises a bacterial toxin.

65. The conjugate of embodiment 1, wherein the payload is or comprises an anti-cancer agent.

66. The conjugate of embodiment 1, wherein the payload is selected from the group consisting of alkylating agents, anti-metabolites, anti-tumor antibiotics, boron neutron capture therapy agents, cell cycle inhibitors, kinesin spindle protein inhibitors, microtubule-binding agents, topoisomerase inhibitors, and combinations thereof.

67. The conjugate of embodiment 1, wherein the payload is or comprises an alkaloid, anthracycline, an auristatin, camptothecin, a folate derivative, a metal complex, a nucleoside analog, a taxane, a vinca alkaloid analog, or a combination thereof.

68. The conjugate of embodiment 1, wherein the payload is or comprises a phytochemical.

69. The conjugate of embodiment 1, wherein the payload is Monomethyl auristatin E (MMAE).

70. The conjugate of embodiment 1, wherein the payload is a detectable entity.

71. The conjugate of embodiment 1, wherein the payload is a small molecule.

72. The conjugate of embodiment 1, wherein the payload is a polypeptide.

73. The conjugate of embodiment 1, wherein the payload is an oligonucleotide.

74. The conjugate of embodiment 73, wherein the polypeptide is an mRNA.

75. The conjugate of embodiment 1, wherein the payload is a particle.

76. The conjugate of embodiment 75, wherein the payload is a lipid nanoparticle.

77. The conjugate of embodiment 1, wherein the payload is a viral capsid.

78. The conjugate of embodiment 1, wherein the payload is a lipid vesicle such as an exosome or liposome.

79. The conjugate of embodiment 1, wherein the payload is or comprises a radioisotope.

80. The conjugate of embodiment 1, wherein the payload is covalently linked to the linker or the peptide by way of a hydroxyl, carboxyl, or amine group.

81. The conjugate of embodiment 1, wherein the payload is or comprises a protein degradation modulator such as a proteasome inhibitor or a PROTAC agent.

82. The conjugate of embodiment 1, wherein the payload is or comprises a nucleic acid editing system such as a CRISPR/Cas, a TALEN, an ADAR, etc.

83. The conjugate of any one embodiments 12-21, wherein the payload is connected to the amino acid residue in position P4.

84. The conjugate of any one embodiments 12-18, wherein the payload is connected to the amino acid residue in position P3.

85. The conjugate of any one embodiments 12-18, wherein the payload is connected to the amino acid residue in position P8.

86. The conjugate of any one embodiments 19-21, wherein the payload is connected to the amino acid residue in position P10.

87. The conjugate of any one of embodiments 1-21, wherein at least one amino acid residue within the sequence of sortilin binding moiety is conjugated with a payload moiety.

88. The conjugate of any one of embodiments 1-21, wherein at least one amino acid residue within the sequence outside of P15, P16 and P17 of sortilin binding moiety is conjugated with a payload moiety.

89. The conjugate of any one of embodiments 1-21, wherein two or more amino acid residues within the sequence of sortilin binding moiety are conjugated with a payload moiety.

90. The conjugate of any one of embodiments 1-21, wherein two or more amino acid residues within the sequence outside of P15, P16 and P17 of sortilin binding moiety is conjugated with a payload moiety.

91. The conjugate of embodiment 1, wherein the linker is cleavable.

92. The conjugate of embodiment 91, wherein the linker is an acid cleavable linker.

93. The conjugate of embodiment 91, wherein the linker is an enzyme cleavable linker.

94. The conjugate of embodiment 1, wherein the linker is a VCPAB linker.

95. The conjugate of embodiment 1, wherein the linker is or comprises an ester, an amide, a hydrozone, a carbonate, a reducible disulfide, and combinations thereof.

96. The conjugate of embodiment 1, wherein the linker is or comprises a thioether, an oxime, a triazole, and combinations thereof.

97. The conjugate of embodiment 1, wherein the linker is redox-sensitive.

98. An engineered sortilin binding peptide comprising
(a) a C-terminal fragment of progranulin, or a variant thereof, which fragment includes not more than about 20 residues corresponding to contiguous C-terminal progranulin residues;
(b) has a length within a range of about 12 to about 20 amino acids and includes a characteristic sequence represented by:

(SEQ ID NO: 1)
(R/N) X²⁻³ (C/L) X⁰⁻¹ R(Q/E/L/B43/B50)
or
(SEQ ID NO: 2)
APRWDAPLRXPALR,

wherein X is any canonical or non-canonical amino acid.

99. An engineered sortilin binding peptide having a length within a range of about 12 to about 20 amino acids and an amino acid sequence that includes a characteristic sequence represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein each X is independently any canonical or non-canonical amino acid; and
at least one X reside is a non-natural amino acid.

100. The peptide of embodiment 98 or 99, wherein the non-natural amino acid is selected from those included in one or more of Tables 2-7.

101. The peptide of embodiment 98 or 99, comprising two cysteine residues within the sequence which form a disulfide bond.

102. The peptide of any one of embodiments 98 or 99, wherein the characteristic sequence is represented by:

(SEQ ID NO: 3)
(R/N) X²⁻³ C X⁰⁻¹ R (Q/E).

103. The peptide of any one of embodiments 98 or 99, wherein the characteristic sequence is represented by:

(SEQ ID NO: 4)
(R/N) X²⁻³ L X⁰⁻¹ R (Q/B43/B50).

104. The peptide of any one of embodiments 98 or 99, wherein the characteristic sequence is represented by:

(SEQ ID NO: 5)
(R/N) X²⁻³ C/L X⁰⁻¹ R Q(L/B13/F02)(L/B13/F02).

105. The peptide of any one of embodiments 98 or 99, wherein the characteristic sequence is represented by:

(SEQ ID NO: 6)
(R/N) X²⁻³ C X⁰⁻¹ R Q (L/B13/F02)(L/B13/F02).

106. The peptide of any one of embodiments 98 or 99, wherein the characteristic sequence is represented by:

(SEQ ID NO: 7)
(R/N) X10 X11 X12 C R Q.

107. The peptide of any one of embodiments 98 or 99, wherein the characteristic sequence is represented by:

(SEQ ID NO: 8)
X4 X5 X6 X7 X8 (R/N) X10 X11 X12 C R Q;

wherein at least one of X4, X5, and X6 is a cysteine residue.

108. The peptide of embodiment 107, wherein the two cysteine residues within the sequence form a disulfide bond.

109. The peptide of any one of embodiments 98 or 99, wherein the characteristic sequence is represented by:

(SEQ ID NO: 9)
(R/E) X4 X5 X6 X7 X8 (R/N) X10 X11 X12 C R Q;

wherein at least one of X4, X5, and X6 is a cysteine residue.

110. The peptide of embodiment 109, wherein the two cysteine residues within the sequence form a disulfide bond.

111. The peptide of any one of embodiments 98 or 99, wherein the characteristic sequence is represented by:

(SEQ ID NO: 10)
(R/E) X4 X5 X6 X7 X8 (R/N) X10 X11 X12 C R Q (L/
B13/F02)(L/B13/F02);

wherein at least one of X4, X5, and X6 is a cysteine residue.

112. The peptide of embodiment 111, wherein the two cysteine residues within the sequence form a disulfide bond.

113. The peptide of any one of embodiments 98 or 99, wherein the characteristic sequence is represented by:

(SEQ ID NO: 11)
C R X10 X11 C X13 R Q;

wherein the two cysteine residues form a disulfide bond and the polypeptide is a cyclic polypeptide.

114. The peptide of any one of embodiments 98 or 99, wherein the characteristic sequence is represented by:

(SEQ ID NO: 12)
(R/H) N X6 X7 C R X10 X11 C X13 R Q;

wherein the two cysteine residues form a disulfide bond and the polypeptide is a cyclic polypeptide.

115. The peptide of any one of embodiments 98 or 99, wherein the characteristic sequence is represented by:

(SEQ ID NO: 13)
(R/H) N X6 X7 C R X10 X11 C X13 R Q (L/B13/F02)
L/B13/F02);

wherein the two cysteine residues form a disulfide bond and the polypeptide is a cyclic polypeptide.

116. The peptide of the preceding embodiments, comprising one or more of:
(i) a basic residue such as L-arginine or an analog thereof at a position corresponding to position P3 and/or P4 of a 17mer C-terminal fragment of progranulin;
(ii) a hydrophobic residue such as a L-tryptophan or an analog thereof at a position corresponding to position P4 of a 17mer C-terminal fragment of progranulin;
(iii) a long hydrophobic residue at a position corresponding to position P10 a 17mer C-terminal fragment of progranulin;
(iv) a covalent bond such as a disulfide bond) between residues at positions corresponding to positions P5 and P13 or P8 and P12 of a 17mer C-terminal fragment of progranulin; and
(v) a hydrophobic residue such as Leucine or a Leucine at a position corresponding to position P16 and/or P17 of a 17mer C-terminal fragment of progranulin.

117. An engineered sortilin binding peptide comprising a sequence of XⁿRDPALRXLL (SEQ ID NO: 14).

118. The peptide of embodiment 117, wherein n is 0.

119. The peptide of embodiment 118, wherein X corresponds a non-natural amino acid selected from those included in one or more of Tables 2-7.

120. The peptide of embodiment 118, wherein X is Q.

121. An engineered sortilin binding peptide comprising a sequence of DDPRAPWPALQRLALRL (SEQ ID NO: 34).

122. A nucleic acid whose nucleotide sequence includes a coding region that encodes a peptide having a length within a range of about 12 to about 20 amino acids and an amino acid sequence that includes a characteristic sequence represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein each X is independently any canonical or non-canonical amino acid.

123. The nucleic acid of embodiment 122, wherein the nucleic acid is DNA.

124. The nucleic acid of embodiment 122, wherein the nucleic acid is RNA.

125. The nucleic acid of embodiment 122, wherein the nucleic acid is mRNA.

126. The nucleic acid of any one of embodiment 122-125, wherein the nucleic acid is part of a viral vector.

127. A host cell comprising the nucleic acid of embodiment 122.

128. A pharmaceutical composition that comprises or delivers a conjugate of any one of embodiments 1-21 or an engineered peptide of embodiment 98 or 99.

129. The pharmaceutical of embodiment 126, which comprises:
an active agent that is or comprises the conjugate of any one of embodiments 1-21 or the engineered peptide of embodiment 98 or 99; and
at least one pharmaceutically acceptable carrier.

130. The pharmaceutical composition of embodiment 129, wherein the payload is an anti-cancer agent.

131. The pharmaceutical composition of embodiment 130, wherein the active agent further comprises an additional anti-cancer agent.

132. A combination comprising:
a conjugate of embodiment 1 or the engineered peptide of embodiment 98 or 99; and
at least one additional therapeutic agent.

133. The combination of embodiment 132, wherein the conjugate and the at least one additional therapeutic agent are included in the same pharmaceutical composition.

134. A combination comprising:
the pharmaceutical composition of embodiment 128; and
at least one additional therapeutic agent.

135. The combination of embodiment 132 or 134, wherein one or both of the payload and the additional therapeutic agent is or comprises an anti-cancer agent.

136. The combination of embodiment 135 wherein the anti-cancer agent is or comprises a small molecule.

137. The combination of embodiment 135, wherein the anti-cancer agent is or comprises an antibody.

138. A method of binding to sortilin, the method comprising a step of:
contacting a system that includes sortilin with a conjugate or an engineered peptide of any one of the foregoing embodiments.

139. A method of inhibiting sortilin, the method comprising a step of:
contacting a system in which sortilin is active with a conjugate or an engineered peptide of any one of the foregoing embodiments.

140. A method of reducing sortilin on cell surfaces, the method comprising a step of:
contacting a system comprising cells with sortilin on their surfaces with a conjugate or an engineered peptide of any one of the foregoing embodiments, the contacting being performed under conditions and for a time sufficient that the sortilin is internalized by the cells.

141. A method for increasing specificity of a therapeutic or diagnostic moiety for a target cell, the method comprising a step of associating the therapeutic moiety to a sortilin binding peptide having a length within a range of about 12 to about 20 amino acids and an amino acid sequence includes a characteristic sequence represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein each X is independently any canonical or non-canonical amino acid.

142. A method for delivering a payload to a sortilin-expressing cell, the method comprising a step of:
contacting the cell with a conjugate of embodiment 1.

143. A method for increasing specificity of a payload for a sortilin-expressing cell, the method comprising a step of:
associating the payload with a polypeptide, wherein the polypeptide includes a sortilin binding moiety that:
has a length within a range of about 12 to about 20 amino acids and includes a characteristic sequence represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein each X is independently any canonical or non-canonical amino acid.

144. A method of increasing cellular uptake of a payload by a target cell, the method comprising a step of:
associating the payload with a polypeptide, wherein the polypeptide includes a sortilin binding moiety that:
(a) corresponds to a fragment of progranulin, or a variant thereof and includes not more than about 20 contiguous residues corresponding to contiguous C-terminal progranulin residues;
(b) has a length within a range of about 12 to about 20 amino acids and includes a characteristic sequence represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein each X is independently any canonical or non-canonical amino acid.

145. The method of any one of embodiments 141-144, wherein the step of associating is or comprises covalently linking.

146. The method of any one of embodiments 141-144, wherein the sortilin binding moiety and therapeutic moiety are covalently associated through a linker.

147. The method of any one of embodiments 141-144, wherein the sortilin-expressing cell is a cancer cell.

148. The method of any one of embodiments 141-144, wherein the sortilin-expressing cell is a mammalian cell.

149. The method of any one of embodiments 141-144, wherein the sortilin-expressing cell is a human cell.

150. A method of treating a subject suffering from a disease, disorder or condition associated with sortilin-expressing cells, the method comprising a step of delivering to the patient a conjugate of embodiment 1.

151. The method of embodiment 150, wherein the subject is suffering from cancer and cancer cells in the subject express a higher level of sortilin compared with reference non-cancerous cells.

152. The method according to embodiment 150 or embodiment 151 wherein the subject is receiving or has received other cancer therapy.

153. The method of embodiment 152, wherein the other cancer therapy is or comprises chemotherapy, cryotherapy, hormone therapy, immunotherapy, radiation therapy, surgery, and combinations thereof.

154. The method of embodiment 150, wherein the step of administering comprises parenterally administering the conjugate.

155. The method of embodiment 150, wherein the patient has a cancer that is a member of the group consisting of breast cancer, colorectal cancer, glioblastoma, lung cancer, ovarian cancer, pancreatic cancer, small intestine cancer, thymus cancer, thyroid cancer, bladder cancer, prostate cancer, kidney cancer, liver cancer, endometrial cancer, skin cancer, stomach cancer and combinations thereof.

156. A method of preparing a conjugate of embodiment 1 or a peptide of embodiment 98 or embodiment 99, the method comprising steps of:
synthesizing the conjugate or peptide using standard solid phase peptide synthesis (SPPS) with Fmoc chemistry;
deprotecting the conjugate or the peptide and clearing the resin; and
purifying the conjugate or peptide using Reverse Phase High Performance Liquid Chromatography.

157. A method of manufacturing a conjugate, the method comprising a step of:
covalently associating a peptide that corresponds to a C-terminal fragment of progranulin, or a variant thereof with a cytotoxic payload, wherein the peptide has an amino acid sequence including a characteristic sequence element represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein each X is independently any canonical or non-canonical amino acid.

158. A method of manufacturing a peptide having a length within a range of about 12 to about 20 amino acids and an amino acid sequence including a characteristic sequence element represented by:

(SEQ ID NO: 1)
(R/N) X²⁻³ (C/L) X⁰⁻¹ R(Q/E/L/B43/B50)
or
(SEQ ID NO: 2)
A P R W D A P L R X P A L R;

wherein X is any canonical or non-canonical amino acid, the method comprising steps of:
synthesizing the peptide using standard solid phase peptide synthesis (SPPS) with Fmoc chemistry;
deprotecting the peptide and clearing the resin; and
purifying the peptide using Reverse Phase High Performance Liquid Chromatography.

159. A method of manufacturing a pharmaceutical composition, the method comprising a step of:
associating an active agent that is or comprises a conjugate or an engineered peptide of any one of the foregoing embodiments with:
at least one pharmaceutically acceptable carrier.

160. The conjugate of any one of embodiments 1-97, wherein the sortilin binding moiety is or comprises the amino acid sequence APRWDAPLRDPALRQLL (SEQ ID NO: 29).

161. The conjugate of any one of embodiments 1-97, wherein the sortilin binding moiety is or comprises the amino acid sequence APRWDAPLRDPALRQ(B13)(G48) (SEQ ID NO: 30).

162. The conjugate of any one of embodiments 160 or 161, wherein the linker is a VCPAB linker.

163. The conjugate of any one of embodiments 160-162, wherein the payload is MMAE.

164. The peptide of any one of embodiments 98-120, comprising a sequence of

(SEQ ID NO: 29)
APRWDAPLRDPALRQLL.

165. The peptide of any one of embodiments 98-120, comprising a sequence of

(SEQ ID NO: 30)
APRWDAPLRDPALRQ(B13)(G48).

166. The nucleic acid of any one of embodiments 122-126, wherein the nucleotide sequence includes a coding region that encodes a peptide comprising a sequence of

(SEQ ID NO: 29)
APRWDAPLRDPALRQLL.

167. The nucleic acid of any one of embodiments 122-126, wherein the nucleotide sequence includes a coding region that encodes a peptide comprising a sequence of

(SEQ ID NO: 31)
APRWDAPLRDPALRQ.

### EXEMPLIFICATION

### Example 1: Materials and Methods

### Peptide Microarray

Synthesis. Peptide microarrays were generated using a SPOT synthesis technique via a MultiPep synthesizer (Intavis). SPOT synthesis of two peptide arrays were conducted on commercially available amino-PEG functionalized cellulose membranes. One array contained linear peptides and the peptides were cyclized via two cysteines. During standard coupling cycles, the peptides were synthesized by using solutions of preactivated amino acids. Non-natural amino acids were in-situ activated using HOBt and DIC. For an efficient coupling of the first amino acid onto the linker group that is necessary for the change of the orientation, the first coupling cycle was carried out using amino acids in-situ activated with DIC. All peptides were bound to the membrane via the N-terminus; the C-terminus was free.

Membranes were prepared to ensure display of free peptide C-terminus: Membranes with the arrays were modified with a glutamic acid. After that modification, membranes were modified with an additional linker molecule that allowed release of built-up peptide chain. After initial peptide synthesis, all peptides were modified with a beta-alanine to achieve more homogeneous coupling behavior. Following build-up of the protected peptide chain, membranes were treated with diluted TFA, so that glutamic acid side chain(s) were available to be coupled to the membrane. Peptides on the arrays were cyclized by formation of an amide bond between the free glutamic acid side chain and the free N-terminus. Cleavage of peptide protecting group(s) on both arrays, as well as release of the free C-terminus on the linear array, was carried out using TFA solutions with scavengers (water, triisopropanol, thioanisole). After the cleavage, membranes were washed, dried and stored at −20° C. until probing. To cyclize the array containing 2 cysteine residues, the array was treated with an aqueous DMSO mixture to achieve formation of disulphide bridge(s) within the peptides. After the treatment, the cyclic peptide array was also dried and stored at −20° C. until probing.

Probing. As a negative control, arrays were probed with secondary antibody (a HRP conjugated rabbit anti-His6 tag (SEQ ID NO: 35) antibody from ICL at a 1:20,000 dilution). The preceding primary probing step was mimicked by using T-TBS during the corresponding incubation time. Detection of the bound secondary antibody was performed using enhanced chemiluminescence (ECL). The ECL scan was performed at a scanning time of 50 s. Eight images of the scanned array were generated during that scanning time. Immediately after the probing, a two-step regeneration of the peptide arrays was carried out (without adding β-mercaptoethanol (BME)) and the membranes were stored at −20° C. until the next probing. As the next step, arrays were probed with SORT1 (Acrobiosystems, SON—H52H5) at a final protein concentration of 30 μg/ml. Detection of bound protein was carried out as described above.

### Peptide Synthesis: SPPS

Peptides were synthesized on Rink amide resin via Fmoc chemistry.

Protecting groups used for amino acids are: t-Butyl group for Ser, Thr, Tyr, Asp and Glu, Trt group for Asn, Cyc, His and Gln, Pbf for Arg, Boc for Lys and Boc.

Each peptide chain was assembled on a resin by repetitive removal of the Fmoc protecting group and coupling of protected amino acid. DIC and HOBt were used as coupling reagents, and NMM was used as base. 20% piperidine in DMF was used as de-Fmoc-reagent. Ninhydrin test was performed after each coupling to check the coupling efficiency.

After removal of the last Fmoc protecting group, the resin was treated with a TFA cocktail for cleavage and removal of side chain protecting groups. Crude peptides were purified by RP-HPLC; peptide fractions with desired purity were lyophilized to generate a powder preparation of purified peptide.

### Dynamic Light Scattering (DLS)

Peptide samples were prepared in 1×PBS, pH 7.4 to a final concentration of 250, 125, 62.5 μM unless otherwise stated. Samples were spun down on a bench top centrifuge, 15 minutes, 15,000 rpm, room temperature, to pellet any aggregated peptide/dust before taking a measurement. Visual inspection of the tubes was used to identify if any large pellets were formed, which is indicative of large aggregates. Clear solutions were then loaded into the DLS plate (Aurora 384-well, black, clear bottom plates (P8806-38403), 20 μL per well), plates were spun 1 min, 4000 rpm to bring the solution to the bottom of the wells. The plate was then transferred and read on the DLS instrument (Wyatt Technology DynaPro Plate Reader III) at 25° C.

### Circular Dichroism (CD)

Peptide samples were diluted into 1×PBS, pH 7.4 to a final concentration of 50 μM unless otherwise stated. Samples were allowed to equilibrate to room temp for 30 min prior to reading and spun on a bench top centrifuge for 15 minutes, 15,000 RPM, room temperature, to pellet any aggregated peptide/dust before taking a measurement. Visual inspection of the tubes was used to identify if any large pellets were formed, which is indicative of large aggregates. A 220 μL aliquot of the peptide sample or buffer blank was added into the CD quartz cuvettes (Jasco J/0556). The samples were then read on a Jasco J-1500 CD machine, at 25° C. with a wavelength scan from 250-190 nM and the raw mdeg values converted to Mean Residue Ellipticity (MRE).

### Fluorescence Polarization (FP)

Relevant peptides (e.g., reference peptide PRGN_WT, negative control peptide PRGN_scr, and peptide PRGN_RL) were synthesized with an N-terminal FAM tag and free C-terminal. Peptides were dissolved into stock solutions in water, then brought up to final concentrations in 1×PBS, pH 7.4 and tested in DLS and CD (methods above) to ensure the peptides were soluble. Peptide concentrations were determined by quantifying the dye using the nanodrop and dye specific extinction coefficient at a specific wavelength. For FAM labeled dyes, the extinction coefficient 83000 cm⁻¹ M⁻¹ was used.

### Direct Binding in FP

FAM labeled peptides were diluted to 1 μM in FP buffer (1×PBS, pH 7.4, 0.1% Tween-20) and serially diluted two-fold for a 12-point series to determine concentration at which the fluorescence intensity was 10,000 RFU and the polarization of the peptide was consistent. A 10× working solution would thus be 10× the concentration of FAM labeled peptide that satisfied the above criteria.

A stock solution of target protein Sortilin (Acrobiosystems, SON—H52H5) was prepared in FP buffer (1×PBS, pH 7.4, 0.1% Tween-20) such that the highest concentration of SORT1 was 3.85 μM. A 12-point, two-fold dilution series of target protein was prepared in a V-bottom 96-well plate for a total volume of 25 μL.

A 10× working solution of FAM-labeled peptides was prepared in the FP buffer (1×PBS, pH 7.4, 0.1% Tween-20). An aliquot of 2.5 μL of the 10× working solution of the FAM-labeled peptides was then added to 22.5 μL of the SORT1 dilution series and pipetted to mix in a V-bottom 96-well plate. A negative control with no target protein was also prepared to assess baseline polarization of the FAM-labeled peptides. The plate was then spun down to pellet the solutions, then 10 μL of the mixture was transferred to an opaque black 384-well non-binding FP plate (Corning® Low Volume 384-well Black Flat Bottom Polystyrene NBS Microplate, #3820) in technical duplicates. The 384-well plate was then centrifuged 1 min, 4000 rpm and fluorescence and polarization of dye measured on a Biotek Synergy Neo2 plate reader using the following settings (Excitation 485/20, Emission 528/20, Top read, Gain: 75, Light source: Xenon Flash, Lamp Energy: Low, Standard Dynamic range, Read speed: Normal, Delay: 0 msec, Measurements/Data Point: 10, Read Height: 9 mm). The data was then fitted to a One Site—Specific Binding curve in GraphPad to calculate the K_{D}.

### Competition Binding in FP.

Competition binding studies were conducted in a similar manner to the direct binding protocol. Unlabeled peptides were prepared with a 12-point, two-fold dilution series in a V-bottom 96-well plate for a total volume of 25 μL with a starting concentration of 50 μM. A 10× working stock of the FAM labeled peptide was prepared as described for the Direct binding experiment.

A 500 nM working solution (EC80) of target protein was prepared in FP buffer (1×PBS, pH 7.4, 0.1% Tween-20). An aliquot of 20 μL of the 500 nM working solution was then added to 2.5 μL of the FAM-peptide, after which 2.5 μL of the unlabeled peptide dilution series was added and mixed by pipetting in a V-bottom 96-well plate. Samples were allowed to incubate for 15 minutes, then 10 μL of the mixture was transferred to an opaque black 384-well non-binding FP plate (Corning® Low Volume 384-well Black Flat Bottom Polystyrene NBS Microplate, #3820) in technical duplicates. The 384-well plate was then centrifuged 1 min, 4000 rpm and fluorescence and polarization of dye measured on a Biotek Synergy Neo2 plate reader using the same settings as for the Direct Binding experiment. Data were then fitted to a One site—Fit logIC50 curve in GraphPad to calculate the IC50.

### Grating-Coupled Interferometry (GCI)

Direct binding affinities of Sortilin-binding peptides were determined using grating coupled interferometry (GCI) assay in two different formats: (a) Immobilized protein as ligand and detection of peptides as analytes in solution and (b) Immobilized peptides as ligands and detection of protein as analyte in solution. Immobilization of protein/peptides as ligands was achieved by capturing the biotinylated ligands to the surface of Streptavidin sensor chips and upon immobilization of the ligands, the interactions between the immobilized ligands and analytes were determined in a multi-cycle kinetics assay setup.

### Immobilization.

For protein immobilization, SORT1 (Acrobiosystems, SON—H52H5) was biotinylated in a 1:1 ratio using NHS-PEG4 Biotin (Thermo Fisher, 21330). Protein was diluted to 0.5 mg/mL in 1×PBS pH 7.4, 10.3% Trehalose and the biotinylation reaction was conducted at 25° C. for 30 min. After biotinylation, unbound biotin was removed via a desalting step (Zeba Spin Desalting Columns (Thermo Fisher) and the ligand protein was brought into the final buffer of 1×PBS pH 7.4, 0.005% Tween-20. Total protein concentration was then determined by measuring absorption at 280 nm using an Implen spectrometer. Biotinylated SORT1 target protein was immobilized as ligand to the surface of a Streptavidin sensor chip (PCH-STA). The chip was first conditioned using a solution of 1 M NaCl, 0.1 M Na-Borate, pH 9.0 at an injection time of 180 see and flow rate of 2.5 μl/min. Ligand capture and immobilization of biotinylated SORT1 (20 μg/ml in running buffer, 1×PBS pH 7.4, 0.005% Tween-20) was performed via Streptavidin capture with an injection time of 2×1200 sec and flow rate of 10 μl/min. To avoid non-specific binding to the remaining, accessible Streptavidin binding pockets, the surfaces were blocked by biocytin injection (10 μg/ml in running buffer), injection time of 60 sec and flow rate of 10 μl/min. Ligand capturing stability was then assessed by rinsing the surface with buffer at high flow rate (100 μL/min). For peptide immobilization, sortilin-binding peptides were synthesized with an N-terminal biotinylation group (e.g. PRGN_WT-biotin, PRGN_RL-biotin). The process of immobilization to the Streptavidin sensor chip was repeated as above.

### Multi-Cycle Kinetics.

Binding of analytes (free peptides or unlabeled SORT1) was performed in a multi-cycle kinetics experiment. Peptide analytes, or SORT1 protein, were diluted into running buffer, 1:2 serial dilution with a maximum concentration of 5000 nM, and were injected in increasing concentrations over ligand and reference surfaces. Raw sensorgrams were inspected for non-specific and ligand-specific analyte binding and the data were double-referenced, solvent corrected and fitted to a 1:1 kinetic model.

### Thermal Unfolding

In order to verify SORT1 protein integrity after biotinylation, a comparative thermal unfolding experiment was performed. For this, the thermal melting temperatures of the non-biotinylated and biotinylated target protein were determined using a Tycho NT.6 nanoDSF instrument with a heating ramp of 30° C./min. For the non-biotinylated reference protein, a single unfolding peak was observed at 66.7° C. For the labeled target protein, a similar unfolding peak at 66.3° C. was observed. Thus, the structural integrity of the target protein was not negatively affected by the biotinylation procedure.

### Murine Serum/Plasma Stability Assay

### Serum

Serum was drawn from male CD-1 mice by Pharmidex. Lyophilized peptides were solubilized in DMSO and then further diluted into male mouse (CD-1) serum at 37° C. in duplicates. The reaction was incubated at 37° C. and samples extracted at 6 time points: 0, 0.16, 0.5, 1, 3, and 6 hrs. Acetonitrile (ACN) with internal standard (tolbutamide) was added to stop the incubation. Samples were centrifuged, and the supernatant was analyzed by HPLC-MS/MS for the parent compound.

### Plasma

Frozen plasma was thawed at 37° C. and centrifuged at 4000 rpm to remove any clots and transferred to 96-well reaction plates by WuXi AppTec. Test compounds were added to the reaction plate at a final concentration of 2 μM and either stopped immediately or incubated at 37° C. for 5 minutes, 10 minutes, 30 minutes, 60 minutes, or 120 minutes. At the end of incubation, stop solution consisting of 200 ng/mL tolbutamide and 200 ng/mL labetalol in ACN was added to precipitate protein and mixed thoroughly. Samples were then transferred to a bioanalysis plate and shaken for 10 minutes prior to HPLC-MS/MS analysis of percent remaining compound.

### Cell-Killing Assay

Anti-proliferative effects of peptides were assessed in a cell viability assay. HCC70 and MDA-MB-231 cells were grown as per the manufacturer's requirements; HCC70 in RPMI 1640+10% FBS+1×PS/AA and MDA-MB-231 in L-15+10% FBS+1×PS/AA. Cells were counted by haemocytometer with Trypan blue staining and the cell concentration was adjusted to desired cell density. 90 μL of cell suspension was added into the assay plates and 90 μL of assay medium into the blank wells. Cells were then incubated overnight at 37° C., 5% CO₂, 95% air and 100% relative humidity for 16-24 hours. Peptides being assessed were dissolved in DMSO and a 400× compound stock plate was prepared to allow for serial dilution of the stock solution. A 10× compound plate was prepared by further diluting the 400× compound stock plate in cell media and added directly to cells to reach final testing concentrations. Cells were returned to the incubator and viability measurements taken following 72 hours. Cell viability was measured using a CellTiter-Glo Luminescent Cell Viability Assay kit (Promega) according to the manufacturer's protocol. Inhibition rate (IR) of the tested compounds was determined using the following formula:

IR(%)=(1−(RLU compound−RLU blank)/(RLU control−RLU blank))*100%

### Cell-Internalization Assay

MDA-MB-231 cells were grown in L-15+10% FBS+1×PS/AA on coverslips inside a Petri dish. When cells have reached the desired confluence (˜80%), growth media was removed and the prewarmed)(37° C. media containing 100 nM LysoTracker was added. Cells were incubated from 30 minutes to 2 hours under growth conditions appropriate for the particular cell type. The LysoTracker-containing medium was removed and cells were incubated with 1 μM Alexa488-tagged PRGN_based peptides for 2 hours, 4 hours and 8 hours. At the indicated time points, cells were analyzed using a confocal fluorescence microscope fitted with the correct filter sets at the described time points.

### CMC and Formulation

Exemplary conjugate agents (e.g., sortilin binding agents), referred to in the present Example as “peptide drug conjugates” or “PDCs” were assessed in a formulation screen to determine an appropriate vehicle for in vivo studies and to assess precipitation risk. PDC solutions were made up to 0.5 mg/mL in the following buffers: 25 mM Histidine 10% sucrose pH 7, 50 mM acetate/acetic acid, 10% sucrose pH 5, 1×PBS pH 7.4, Saline, 50 mM HEPES 10% sucrose pH 7. Solubility was assessed using a shake flask method and the test compound concentration of the filtrate was confirmed using HPLC-UV.

### Maximum Tolerated Dose (MTD) Study

BALB/c Nude mice, female, 6-8 weeks, weighing approximately 18-22 g were purchased from the Shanghai SLAC Laboratory Animal Co., LTD. MTD studies were conducted at WuXi AppTec and the care and use of animals was conducted in accordance with the regulations of the Association for Assessment and Accreditation of Laboratory Animal Care (AAALAC). Two MTD studies were performed (a) a low dose study with doses ranging from 0.1, 0.5, 1 and 3 mg/kg and (b) a high dose study with doses ranging from 3, 5, 10 mg/kg. Dosing volume was determined based on body weight (10 ul/g) and PDCs were dissolved in 25 mM Histidine 10% sucrose pH 7 and administered i.v. at a single dose at day 0. The treatment and observation were conducted up to 7 days and MTD was defined by ≥20% body weight loss. Body weights were measured daily, death and observed clinical signs were recorded.

### Murine Xenograft

Murine xenograft experiments were conducted at WuXi AppTec and/or Charles River Laboratories and the care and use of animals was conducted in accordance with the regulations of the Association for Assessment and Accreditation of Laboratory Animal Care (AAALAC). MDA-MB-231 tumor cells (ATCC, Manassas, VA, cat #HTB-26) were grown and maintained in L15 medium supplemented with 10% heat inactivated fetal bovine serum, 1% Antibiotic-Antimycotic, and L-glutamine (2 mM) at 37° C. in an atmosphere in 5% CO₂, 95% air. BALB/c Nude, female, 6-8 weeks, weighing approximately 18-22 g were purchased from Vital River Laboratory Animal Technology Co., LTD. Mice were inoculated subcutaneously at the right flank with MDA-MB-231 tumor cells (e.g., triple negative breast cancer cells) (10×10⁶) in 0.2 mL of PBS with Matrigel (1:1) for tumor development. Animals were randomized and treatment was started when the average tumor volume reached approximately 150-200 mm³ for the efficacy study. Dosing volume was adjusted based on body weight and was administered at 10 ml/Kg. PRGN_WT_PDC, PRGN_RL_PDC, Arb-SAR-Q15-PDC, Arb_cyclic_G23-PDC, PRGN_scr_PDC were dissolved in 25 mM Histidine 10% sucrose pH 7 and one or more of the following doses, 3, 1, 0.3 mg/kg, were administered to the mice i.v. at a QW×4 schedule. Free MMAE payload was also tested, and similarly dissolved in 25 mM Histidine 10% sucrose pH 7 and one or more of the following doses, 0.6, 0.06 mg/kg, were administered to the mice i.v. at a QW×4 schedule. Tumor sizes were measured twice weekly in two dimensions using a caliper, and the volume in mm³ calculated using the formula: V=0.5 a×b², where a and b are the long and short diameters of the tumor, respectively. TGI was calculated for each group using the formula: TGI (%)=[1−(Ti−T0)/(Vi−V0)]×100; where Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment.

### Surface Plasmon Resonance (SPR)

SPR analysis was performed on Series S CM5 chips (Cytiva) using a Biacore 8K instrument (Cytiva). Sensorgrams were double-referenced by subtracting the response on both a reference flow cell and a blank sample. Human SORT1 (78-755)-His (Acrobiosystems) was diluted to 60 μg/mL in 10 mM sodium acetate pH 4.5 buffer and immobilized onto Series S CM5 chips using amine coupling at a flow rate of 10 μL/min for 45 s. The final immobilization level of SORT1 was between 2200-2600 RU.

For the multi-cycle kinetics analysis runs, a running buffer of 1×PBS pH 7.4, 0.005% Tween-20 was used. Five start-up cycles and a blank cycle preceded each analysis run. The runs were performed by sequential injections of analyte (0-25 nM) with a 150 seconds (s) association and 300 s dissociation at a flow rate of 30 μL/min, followed by a regeneration with 2 M MgCl₂ for 200 s at a flow rate of 10 μL/min. The kinetic rate constants (kₐ and k_{d}) and the equilibrium binding constant (K_{d}) were calculated using a 1:1 kinetic binding model on the Biacore Insight Evaluation Software (Cytiva).

### Murine Hematotoxicity Assay

Murine hematotoxicity experiments were conducted at Charles River Laboratories and the care and use of animals was conducted in accordance with the regulations of the Association for Assessment and Accreditation of Laboratory Animal Care (AAALAC). Wild type BALB/c, female, 7-8 weeks, weighing approximately 18-22 g were randomized and dosed intravenously with control or test articles at a QW×4 schedule. Dosing volume was adjusted based on body weight and was administered at 10 mL/Kg. PDCs were dissolved in 25 mM Histidine 10% sucrose pH 7 and 3 mg/kg was administered. Free MMAE payload was also tested, and similarly dissolved in 25 mM Histidine 10% sucrose pH 7 and administered to the mice at a dose of 0.6 m/kg by i.v. injection at a QW×4 schedule. 3 days following each dose, 150 μL of whole blood was collected via submandibular method and placed in K2 EDTA tubes stored on ice. Blood neutrophil counts were performed on the day of collection by IDEXX labs. Body weights were recorded twice a week until study endpoint.

### Example 2—Homing Peptides Against SORT1

The present Example describes development of certain Sortilin-binding peptides (referred to in the present Example as “homing peptides”) useful in accordance with the present disclosure.

Specifically, we synthesized certain Sortilin-binding peptides based on the C-terminal region of progranulin (Zheng, Y., PlosOne, 2011; Hu, F., Neuron, 2010).

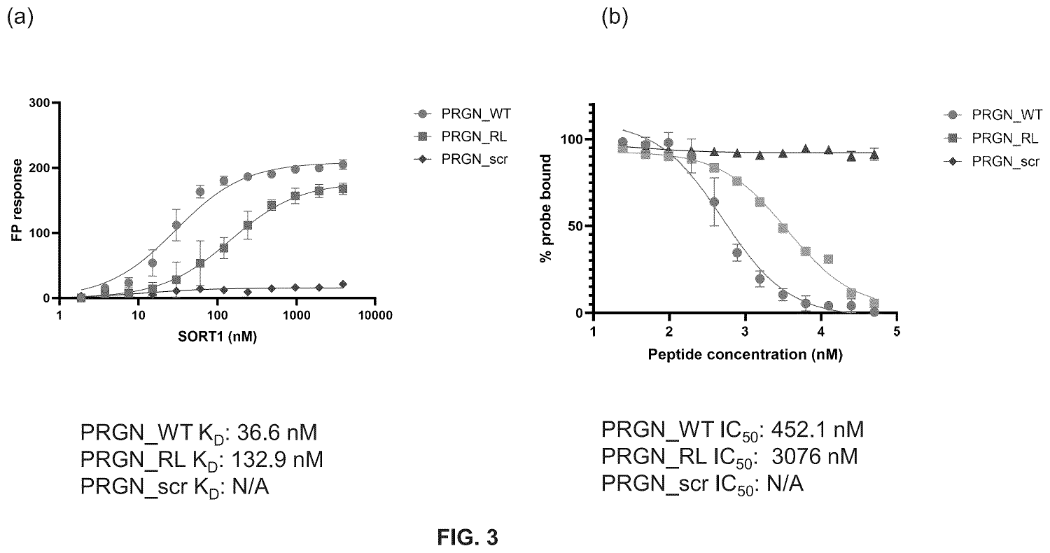

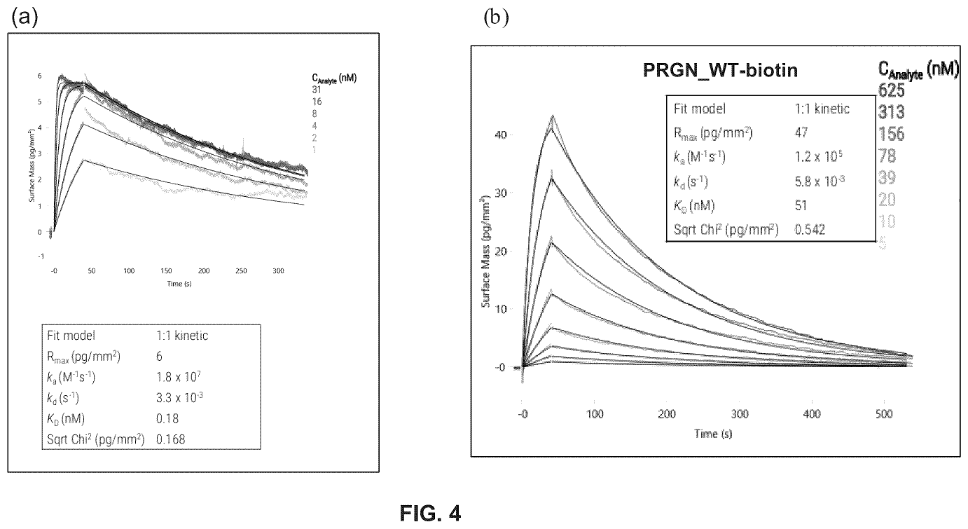

A reference peptide, called “PRGN_WT” was synthesized with the amino acid sequence APRWDAPLRDPALROLL-COOH (SEQ ID NO: 29), which is the 17 C-terminal residues of human progranulin, and was confirmed to bind to SORT1-Ag with high affinity in direct binding experiments. Specifically, PRGN_WT was shown to bind to SORT1-Ag with an affinity of 36.6 nM in FP and 51 nM in GCI (FIGS. 3a and 4b).

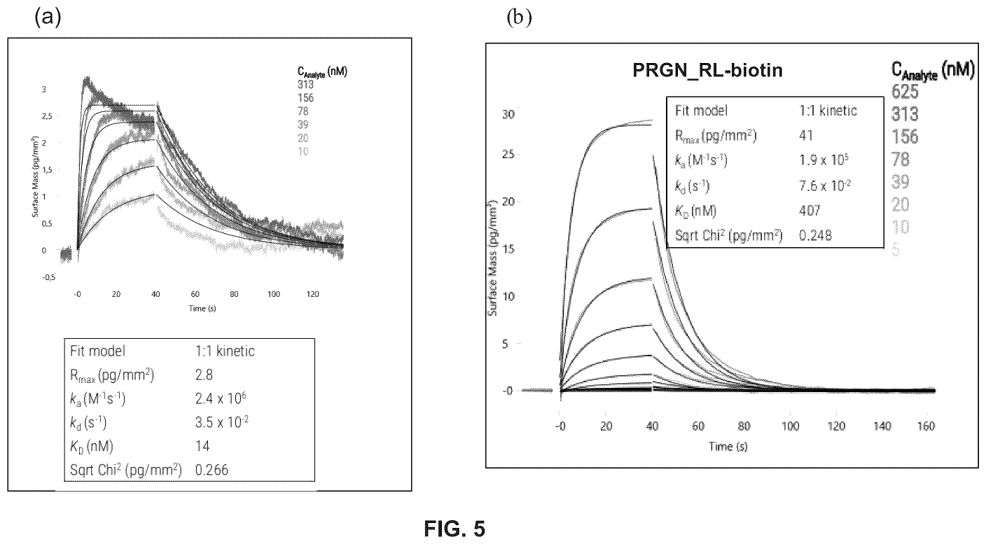

To better understand the effects of affinity (e.g. low nM affinity vs high nM affinity) in downstream efficacy and toxicity studies, we developed a derivative peptide that we called “PRGN_RL”, with the amino acid sequence DDPRAPWPALQRLALRL (SEQ ID NO: 34). PRGN_RL was determined to bind to SORT1-Ag with ˜7× poorer affinity than the WT sequence (i.e., than PRGN_WT). Specifically, PRGN_RL was determined to bind to SORT1-Ag with 132.9 nM affinity in FP and 407 nM in GCI (FIGS. 3a and 5b).

As a negative control, we synthesized a reference peptide, which we called “PRGN_scr”, whose amino acid sequence was a scrambled version of the sequence of PRGN_WT. Specifically, the amino acid sequence of PRGN_scr was QRLARLPRDLLAWPPAD (SEQ ID NO: 36); this peptide was shown to have no binding in the FP assay.

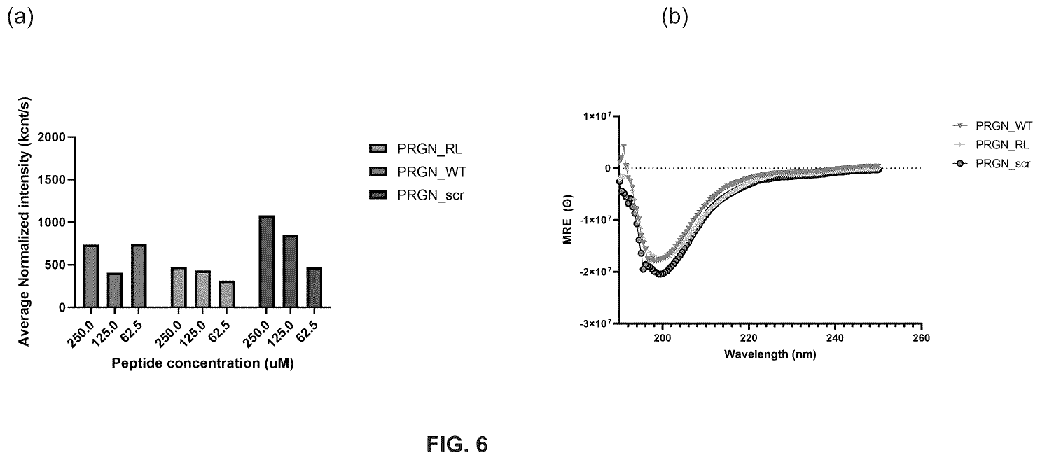

In-depth biophysical characterization by DLS and CD further showed that all three synthesized peptides (i.e., PRGN_WT, PRGN_RL, and PRGN_scr) demonstrated good solubility at the working concentrations (250 nM, PBS, pH 7.4) and exhibited a random coil secondary structure (FIG. 6b).

Stability of these PRGN_based peptides was further assessed in a murine serum stability assay, with PRGN_WT and PRGN_RL demonstrating half-lives of T1 2=3.16 and 27.7 min respectively. Additional peptides described herein were designed and developed, among other things, to have improved stability characteristics (e.g., in a murine serum stability assay) relative to one or more of PRGN_WT, PRGN_RL, and PRGN_scr, with particular focus on improved such stability relative to PRGN_WT.

### Example 3—Biophysical Characterization of PRGN Based PDCs

The present Example describes certain conjugate agents (e.g., peptide-drug conjugates (PDCs)) using provided sortilin-binding peptides, and certain biophysical characterization thereof.

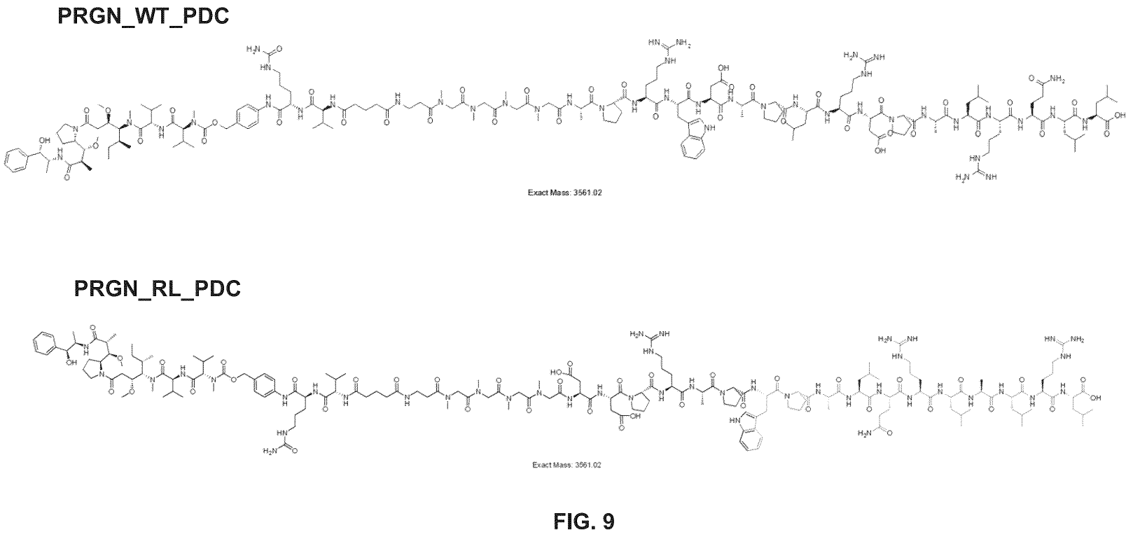

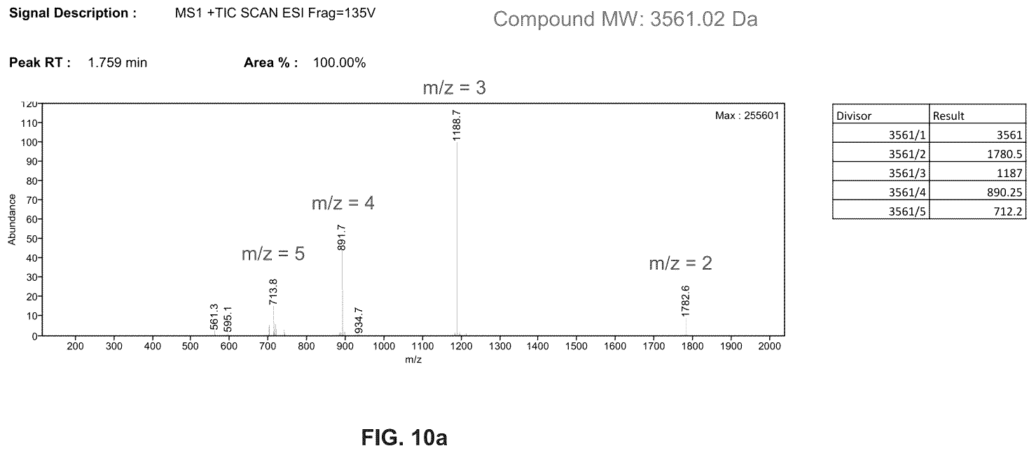

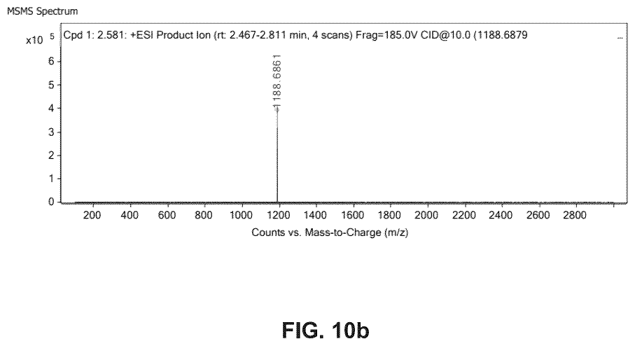

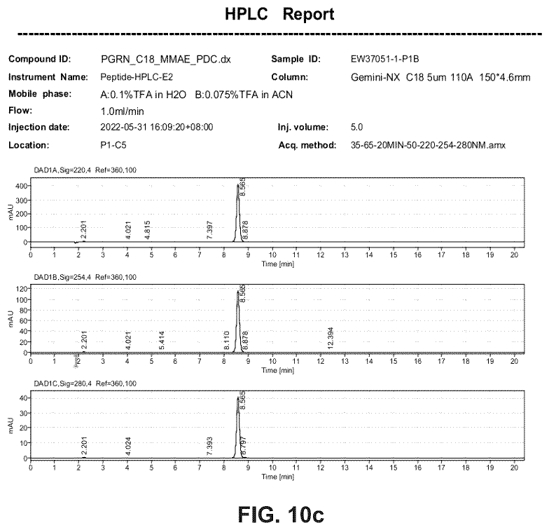

We converted certain homing peptides (e.g., certain sortilin-binding peptides and/or certain reference peptides) into peptide Drug Conjugates (PDCs), specifically by conjugating the cytotoxic drug MMAE via a valine-citruline VCPAB linker at the N-terminus of a homing peptide to create a PDC. The structure of PRGN_WT_PDC is shown in FIG. 9. Additional homing peptides PRGN_RL and PRGN_scr were chemically functionalized to PDCs in the same manner. PDCs were synthesized to a purity of 95% as assessed by MS-MS, LC-MS and HPLC for downstream experiments (FIG. 10).

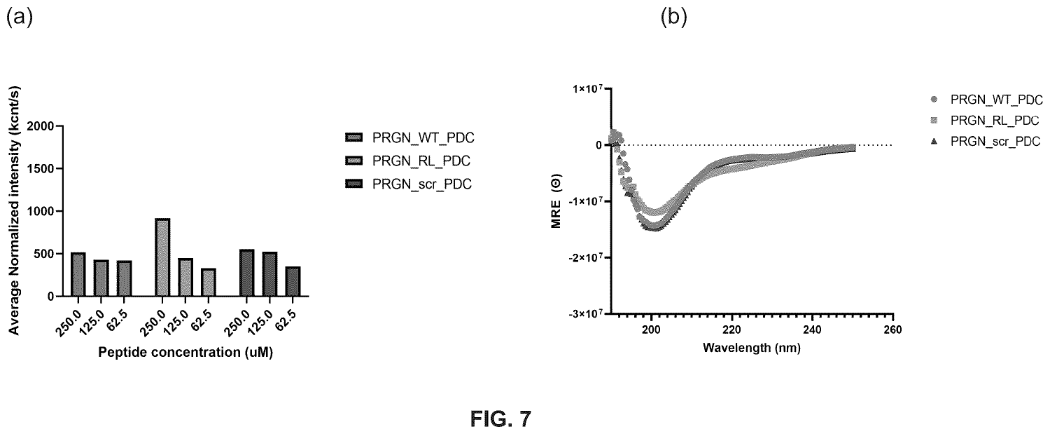

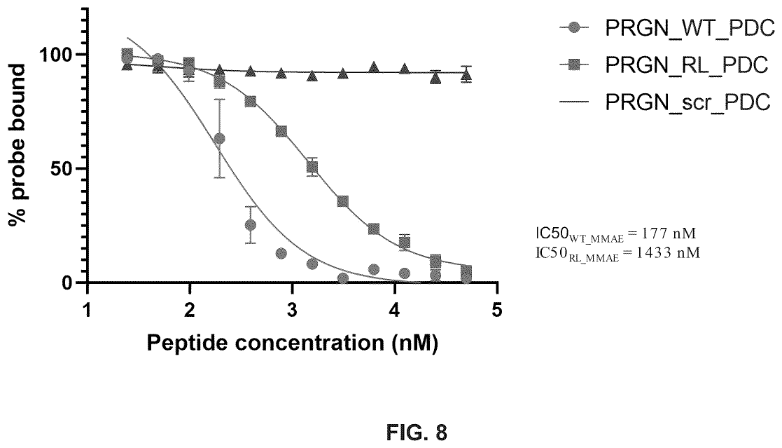

PDCs were re-characterized in certain biophysical assays (specifically, in this Example, DLS and CD), and were demonstrated to remain soluble and well behaved in DLS and in a random coil secondary structure (FIG. 7b). Characterization in FP competition binding assays indicated a ˜2.5× increase in affinity for both PRGN_WT_PDC and PRGN_WT_PDC relative to their parent homing peptides (FIG. 8), while the negative control PRGN_scr_PDC continued to exert no binding. Additional functional assays were then performed with these PDCs.

### Example 4—PRGN Based PDCs Kill SORT1 Expressing Cancer Cells

The present Example demonstrates performance of certain provided PRGN_based PDCs in a cancer-cell killing assay using cancer cells that overexpress sortilin.

Breast cancer cell-lines HCC70 and MDA-MB-231 were chosen based on their high SORT1 gene expression levels.

Both PRGN_WT_PDC and PRGN_WT_RL potently kill sortilin overexpressing cancer cells (IC50=20.8 nM and 111.9 nM respectively in MDA-MB-231 cells), with PRGN_WT_PDC exhibiting a ˜5-fold greater potency compared to PRGN_RL_PDC (FIG. 11). The difference in potency between PRGN_WT_PDC and PRGN_RL_PDC correlates well with the difference in affinity for SORT1, supporting a SORT1 specific mechanism.

### Example 5—PRGN Based PDCs are Well Tolerated in a Murine MTD Study

### CMC Assessment

The present Example demonstrates, among other things, the assessment of the vehicle of choice for the in vivo studies and to assess precipitation risk, by testing certain provided PDCs in a formulation buffer screen. The following buffers were screened: 25 mM Histidine 10% sucrose pH 7, 50 mM acetate/acetic acid, 10% sucrose pH 5, 1×PBS pH 7.4, Saline, 50 mM HEPES 10% sucrose pH 7. The PDCs were soluble in all buffers tested (Table 1), and we proceed to use 25 mM Histidine, 10% sucrose pH 7 as our buffer of choice for our in vivo studies.

TABLE 1
CMC characterization of PRGN_based PDCs and buffer
screening. Highlighted in yellow are buffers of choice.

| PDC | conc | buffer | result |
|---|---|---|---|
| PRGN_WT_PDC | 0.5 mg/mL | 25 mM Histidine | clear solution |
| | | 10% sucrose pH 7 | |
| | 0.5 mg/mL | 50 mM acetate/ | clear solution |
| | | acetic acid, 10% | |
| | | sucrose pH 5 | |
| | 0.5 mg/mL | 1x PBS pH 7.4 | clear solution |
| | 0.5 mg/mL | Saline | clear solution |
| | 0.5 mg/mL | 50 mM HEPES 10% | clear solution |
| | | sucrose pH 7 | |

### Murine MTD Study

The present example demonstrates, among other things, identification of the maximum tolerated dose (MTD) of certain provided PRGN_based PDC as a single reagent in naïve female BALB/c nude mice. Doses of 3, 5, and 10 mg/kg were administered. MTD was defined by ≥20% body weight loss and body weight measured daily.

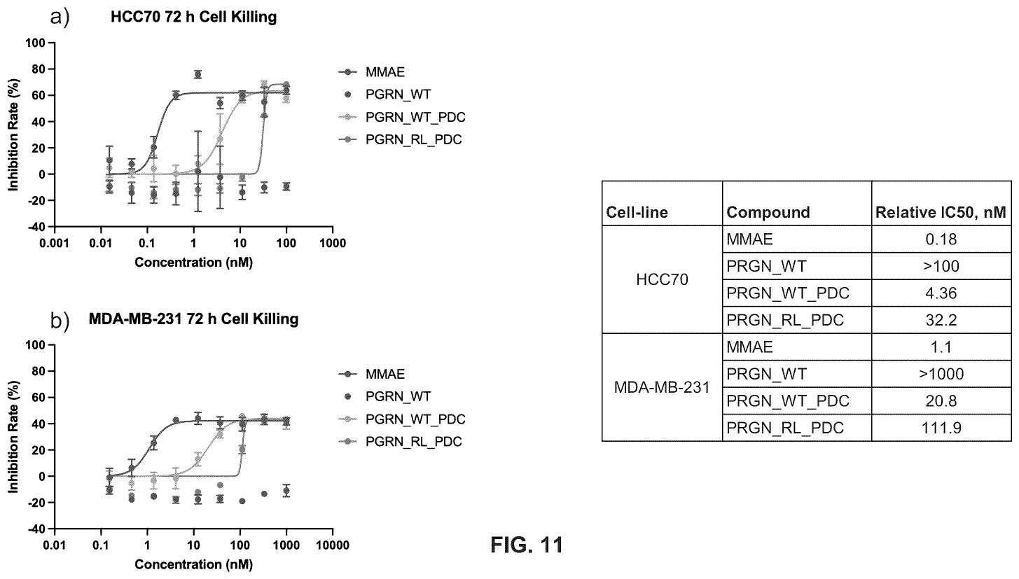

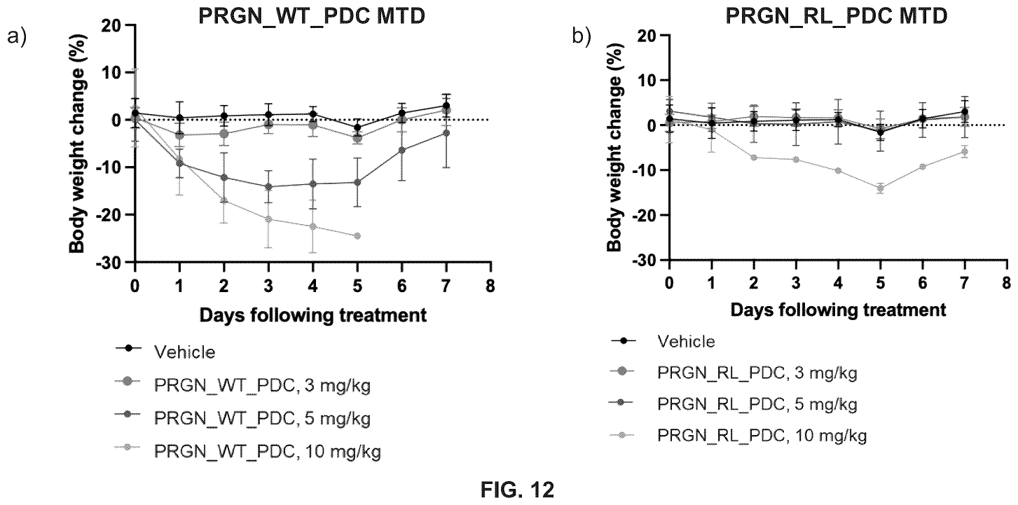

We observed that the higher doses of PRGN_WT_PDC demonstrated significant toxicity (FIG. 12c, P<0.0001 by Two-way Anova). At 10 mg/kg treatment of PRGN_WT_PDC, mice exhibited significant toxicity and greater than 20% body weight loss after 48 hrs of treatment and were euthanized after 96 hr of treatment. At 5 mg/kg treatment of PRGN_WT_PDC, mice exhibited toxicity and a sustained body weight loss of ˜15% after 48 hrs of treatment (P=0.029), which was then recovered at Day 7.

PRGN_RL_PDC demonstrated mild toxicity at the highest dose of 10 mg/kg. Without wishing to be bound by any particular theory, we propose that the observed relatively mild toxicity may be attributable the significantly lower affinity of the homing peptide for SORT1.

### Example 6—PRGN Based PDCs Tested in a Murine Xenograft Study

The present Example demonstrates effects of certain provided PRGN_based PDC in a MDA-MB-231 xenograft efficacy model. PRGN_WT_PDC was dissolved in 25 mM Histidine 10% sucrose pH 7 and administered to mice at 3, 1, 0.3 mg/kg, i.v. at a QW x 4 schedule.

To characterize the effects of the MMAE payload alone, we included free MMAE at molar equivalency to PDCs, and tested at 0.6 and 0.06 mg/kg, as separate arms.

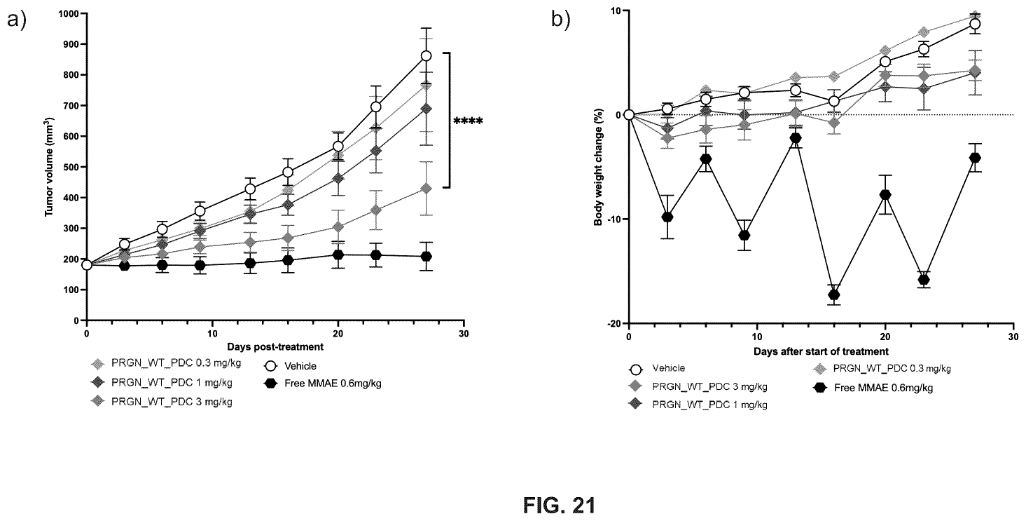

In the xenograft efficacy model, we observed dose-dependent tumor growth inhibition with PRGN_WT_PDC (FIG. 21a, P<0.0001 by Two-way Anova). At 3 mg/kg treatment of PRGN_WT_PDC, tumor growth was inhibited by 63.45% relative to vehicle at day 27 (FIG. 21a).

We sought to evaluate the toxicity of PRGN_WT_PDC relative to free MMAE by comparing body weight loss in the MDA-MB-231 xenograft model. At each dose of PRGN_WT-PDC, body weight variation mirrored that observed with vehicle treatment (FIG. 21b). Free MMAE administered at equivalency to 3 mg/kg PRGN_WT_PDC resulted in body weight fluctuations corresponding to treatment times.

Without wishing to be bound by theory, we propose that the low toxicity observed with PRGN_WT_PDC may result form specific targeting of SORT1-expressing MDA-MB-231 in the xenograft model, whereas the body weight fluctuations caused by the equivalent dose of MMAE may result from non-specific targeting of the free drug.

### Example 7—Exemplary Provided Peptides

The present Example describes certain Sortilin-binding peptides provided by the present disclosure. Among other things, the present disclosure provides an insight that non-natural amino acids may be particularly useful in sortilin-binding peptides (e.g., specifically in sortilin-binding polypeptides corresponding to a C-terminal progranulin fragment); peptides exemplified in the present Example include such non-natural amino acids.

Two different scaffolds, linear and cyclic, are utilized in these exemplified peptides. About ˜600 linear and ˜600 cyclic peptides were designed; each of which incorporates one or more non-natural amino acids.

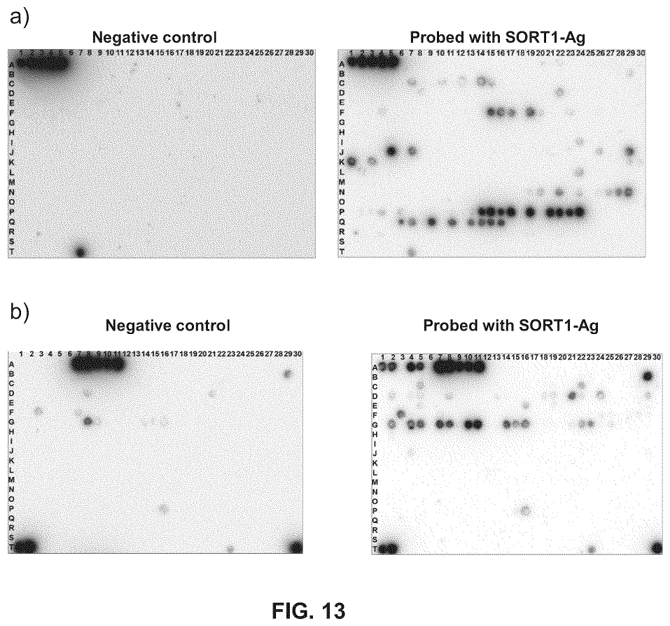

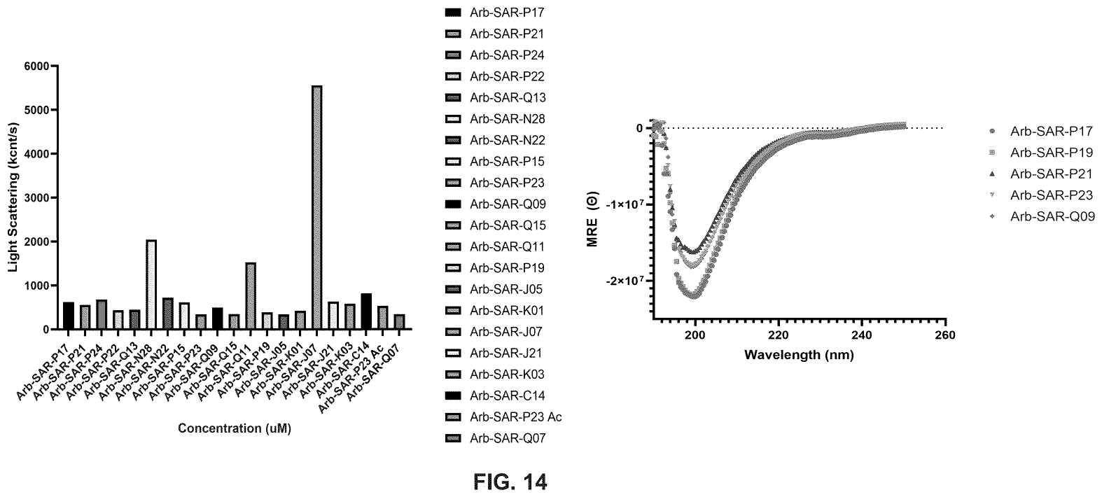

Peptides were assessed for their binding to SORT1-Ag in a peptide microarray screen (FIG. 13 and FIG. 14). As a negative control, the peptides arrays were first probed with the secondary antibody (a HRP conjugated rabbit anti-His6 tag antibody (SEQ ID NO: 35) from ICL at a 1:20,000 dilution). The peptide array containing the linear designs indicated low levels of non-specific binding when probed with the negative control (FIG. 13a). In the array containing the cyclic designs, some sequences demonstrated non-specific binding to the negative control secondary antibody, notably positions G8, B29, T23 etc (FIG. 13b). Peptides that demonstrated non-specific binding were excluded from further assessments and were not scored as positive hits.

After non-specific binding was tested, peptides that specifically bind to sortilin were identified by probing the arrays with with 30 μg/ml of SORT1 antigen. Sortilin-specific hits were identified in both arrays. Specifically, 19 linear peptides and 15 cyclic peptides set forth in Table 2 were determined to show sortilin-specific binding:

TABLE 2
Certain exemplary SORT1 binding peptides and their non-natural amino acid
codes. Table discloses SEQ ID NOS 37-44, 30, 45-49, 23 and 50-71,
respectively, in order of appearance.
Note that PRGN_RL, having the amino acid sequence DDPRAPWPALQRLALRL
(SEQ ID NO: 34) is referred to as “Arb_C18_RL” in Table 2 below.

| Index | Name | N-term | :Sequence | C-term | Cyclized | Type |
|---|---|---|---|---|---|---|
| 1 | PRGN WT | Ac | APRWDAPLRDPALRQLL | COOH | linear | homing peptide |
| 2 | Arb_C18_RL | Ac | DDPRAPWPALQRLALRL | COOH | linear | homing peptide |
| 3 | Arb-SAR-P21 | NH2 | APRWDAPLRDPALRQ | COOH | linear | homing peptide |
| | | | (F01)(F02) | | | |
| 4 | Arb-SAR-P24 | NH2 | APRWDAPLRDPALRQ | COOH | linear | homing peptide |
| | | | (G48)(F02) | | | |
| 5 | Arb-SAR-P22 | NH2 | APRWDAPLRDPALRQ | COOH | linear | homing peptide |
| | | | (F02)(F02) | | | |
| 6 | Arb-SAR-P19 | NH2 | APRWDAPLRDPALRQ | COOH | linear | homing peptide |
| | | | (A45)(F02) | | | |
| 7 | Arb-SAR-P23 | NH2 | APRWDAPLRDPALRQ | COOH | linear | homing peptide |
| | | | (B13)(F02) | | | |
| 8 | Arb-SAR-Q09 | NH2 | APRWDAPLRDPALRQ | COOH | linear | homing peptide |
| | | | (B43)(G48) | | | |
| 9 | Arb-SAR-Q15 | NH2 | APRWDAPLRDPALRQ | COOH | linear | homing peptide |
| | | | (B13)(G48) | | | |
| 10 | Arb-SAR-Q13 | NH2 | APRWDAPLRDPALRQ | COOH | linear | homing peptide |
| | | | (F01)(G48) | | | |
| 11 | Arb-SAR-N22 | NH2 | APRWDAPLRDPALRQ | COOH | linear | homing peptide |
| | | | (B43)(B43) | | | |
| 12 | Arb-SAR-P15 | NH2 | APRWDAPLRDPALRQF | COOH | linear | homing peptide |
| | | | (F02) | | | |
| 13 | Arb-SAR-Q7 | NH2 | APRWDAPLRDPALRQF | COOH | linear | homing peptide |
| | | | (G48) | | | |
| 14 | Arb-SAR-J05 | NH2 | APRWDAPLRDPALRQL | COOH | linear | homing peptide |
| | | | (F02) | | | |
| 15 | Arb-SAR-K01 | NH2 | APRWDAPLRDPALR | COOH | linear | homing peptide |
| | | | (B43)LL | | | |
| 16 | Arb-SAR-K3 | NH2 | APRWDAPLRDPALR | COOH | linear | homing peptide |
| | | | (B50)LL | | | |
| 17 | Arb-SAR-C14 | NH2 | APRWDAPLRFPALRQLL | COOH | linear | homing peptide |
| 18 | Arb-SAR-J21 | NH2 | APRWDAPLRDPALRQ | COOH | linear | homing peptide |
| | | | (F02)L | | | |
| 19 | Arb-SAR-P17 | NH2 | APRWDAPLRDPALRQ | COOH | linear | homing peptide |
| | | | (B43)(F02) | | | |
| 20 | Arb-SAR-P23-Ac | Ac | APRWDAPLRDPALRQ | COOH | linear | homing peptide |
| | | | (B13)(F02) | | | |
| 21 | Arb_cyclic_G10 | Ac | R | COOH | disulfide_ | homing peptide |
| | | | (Nle)CGPIRSPACRQ | | cyclized | |
| | | | (B13)(F02) | | | |
| 22 | Arb_cyclic_G7 | Ac | RGCIPRRQDACRQ | COOH | disulfide_ | homing peptide |
| | | | (B13)(F02) | | cyclized | |
| 23 | Arb_cyclic_G5 | Ac | RDCVSLR | :COOH | disulfide_ | homing peptide |
| | | | (Nle)HVCRQ | | cyclized | |
| | | | (B13)(F02) | | | |
| 24 | Arb_cyclic_G4 | Ac | RDCPSLRAKVCRQ | COOH | disulfide_ | homing peptide |
| | | | (B13)(F02) | | cyclized | |
| 25 | Arb_cyclic_G14 | Ac | RSCTTHR | COOH | disulfide_ | homing peptide |
| | | | (Nle)ATCRQ | | cyclized | |
| | | | (B13)(F02) | | | |
| 26 | Arb_cyclic_D21 | Ac | RNFHCRAQC | COOH | disulfide_ | homing peptide |
| | | | (Nle)RQ | | cyclized | |
| | | | (B13)(F02) | | | |
| 27 | Arb_cyclic_G2 | Ac | RDCASLRAPPCRQ | COOH | disulfide_ | homing peptide |
| | | | (B13)(F02) | | cyclized | |
| 28 | Arb_cyclic_G16 | Ac | RVCANHR | COOH | disulfide_ | homing peptide |
| | | | (Nle)NTCRQ | | cyclized | |
| | | | (B13)(F02) | | | |
| 29 | Arb_cyclic_F3 | Ac | EWCGLHNHPTCRQ | COOH | disulfide_ | homing peptide |
| | | | (B13)(F02) | | cyclized | |
| 30 | Arb_cyclic_G23 | Ac | RWCENRR | COOH | disulfide_ | homing peptide |
| | | | (Nle)FTCRQ | | cyclized | |
| | | | (B13)(F02) | | | |
| 31 | Arb_cyclic_G15 | Ac | RTCNHLRNPACRQ | COOH | disulfide_ | homing peptide |
| | | | (B13)(F02) | | cyclized | |
| 32 | Arb_cyclic_G22 | Ac | RWCENQR | COOH | disulfide_ | homing peptide |
| | | | (Nle)TVCRQ | | cyclized | |
| | | | (B13)(F02) | | | |
| 33 | Arb_cyclic_C22 | Ac | HNFYCRKHCQRQ | COOH | disulfide_ | homing peptide |
| | | | (B13)(F02) | | cyclized | |
| 34 | Arb_cyclic_D24 | Ac | RNIFCRK | COOH | disulfide_ | homing peptide |
| | | | (Nle)CERQ | | cyclized | |
| | | | (B13)(F02) | | | |
| 35 | Arb_cyclic_C5 | Ac | AQC | COOH | disulfide_ | homing peptide |
| | | | (Nle)GRNRACRQ | | cyclized | |
| | | | (B13)(F02) | | | |
| 36 | Arb_cyclic_K9 | Ac | pCeklqRvrTCrE | COOH | disulfide_ | homing peptide |
| | | | (B13)(F02) | | cyclized | |
| 37 | Arb_linear_Q10 | Ac | RDPALR | COOH | disulfide_ | homing peptide |
| | | | (B43)LL | | cyclized | |

| Abbreviation | Definition |
|---|---|
| (F01) | L-3-Cyclopropyl-Alanine, N[C@@H](CC1CC1)C(O)O |
| (F02) | L-4-Methyl_sucine, N[C@@H](CC(C)(C)(C)C(═O)O) |
| (B43) | L.Homoleucine. CC(C)CC[C@@H](C(═O)O)N |
| (G48) | 3-cyclobutyl alanine, C1CC(C1)C[C@@H](C(═O)O)N |
| (A45) | L Norleucine, CCCC[C@@H](C(═O)O)N |
| (B13) | L-3-Methyl-Valine, CC(C)(C)[C@@H](C(═O)O)N |
| (B50) | L-homoserine, C(CO)C(C(═O)O)N |
| Nle | L.Norleucine, CCCC[C@@H](C(═O)O)N |
| SAR | Sarcosine, CNCC(═O)O |
| bAla | beta-Alanine, C(CN)C(═O)O |
| Ac | Acetyl group |
| Amide | Amide group |

As can be seen, these peptides are characterized by a common sequence element:

(SEQ ID NO: 1)
(R/N) X²⁻³ (C/L) X⁰⁻¹ R (Q/E/L/B43/B50)
or
(SEQ ID NO: 2)
A P R W D A P L R X P A L R;

wherein each X is independently any canonical or non-canonical amino acid.

Certain of these peptides are characterized by a common sequence:
(R/N) X²⁻³ CX⁰⁻¹ RQ(L/B13/F02)(L/B13/F02)

Certain of these peptides are characterized by a common sequence:
(R/N) X10 X11 X12 CR Q

Certain of these peptides are characterized by a common sequence:
X4 X5 X6 X7 X8 (R/N) X10 X11 X12 CR Q
   wherein at least one of X4, X5, and X6 is a cysteine residue.

Certain of these peptides are characterized by a common sequence:
(R/E) X4 X5 X6 X7 X8 (R/N) X10 X11 X12 CR Q
   wherein at least one of X4, X5, and X6 is a cysteine residue.

Certain of these peptides are characterized by a common sequence:
(R/E) X4 X5 X6 X7 X8 (R/N) X10 X11 X12 CR Q (L/B13/F02) (L/B13/F02)
   wherein at least one of X4, X5, and X6 is a cysteine residue.

Certain of these peptides are characterized by a common sequence:

(SEQ ID NO: 11)
C R X10 X11 C X13 R Q

wherein the two cysteine residues form a disulfide bond and the polypeptide is a cyclic polypeptide.

Certain of these peptides are characterized by a common sequence:

(SEQ ID NO: 12)
(R/H) N X6 X7 C R X10 X11 C X13 R Q

wherein the two cysteine residues form a disulfide bond and the polypeptide is a cyclic polypeptide.

Certain of these peptides are characterized by a common sequence:

(SEQ ID NO: 13)
(R/H) N X6 X7 C R X10 X11 C X13 R Q (L/B13/F02)
(L/B13/F02)

wherein the two cysteine residues form a disulfide bond and the polypeptide is a cyclic polypeptide.

Alternatively or additionally, cyclic peptides in Table 2 can be characterized by the following sequence patterns:

Cyclic group 1: Has a length of 14 or 15 amino acids, a cysteine residue at P13 that is disulfide bonded to a cysteine residue at P4, P5 or P6. The sequence pattern is:
(R/E/X)(X/C)(C/X)(X/C)XX(R/N)XXXCRQ(L/X)(L/X)

Cyclic group 2: Has a length of 14 amino acids, a cysteine residue at P12 that is disulfide bonded to a cysteine residue at P8. The sequence pattern is:

(SEQ ID NO: 72)
(R/H/X)NXXCRXXCXRQ(L/X)(L/X),

wherein each X is independently any canonical or non-canonical amino acid.

Particular exemplified linear peptides have an amino acid sequence as set forth below:

(SEQ ID NO: 73)
APRWDAPLR(D/F/X)PALR(Q/X)(L/X)(L/X).

Thus, up to four positions are varied in these exemplified 17mer linear sequences relative to that of progranulin. These particular exemplified peptides therefore have an amino acid sequence that is at least 76% identical to that of a corresponding fragment of PGRN (or to PGRN-SAR-Q15).

Without wishing to be bound by any particular theory, we propose that the C-terminal part of the sequence, R(D/F/X)PALR(Q/X)(L/X)(L/X) (SEQ ID NO: 19), is responsible for sortilin binding, and, for example, contains the major driver for binding affinity. We further propose that the N-terminal part of the sequence, APRWDAPL (SEQ ID NO: 20), may bind to a secondary site in the target, contributing to the binding affinity but to a lesser extent.

Having considered 3D structural data, we have designed additional peptides, which may have further affinity and property improvements. Specifically, we have designed peptides (e.g., linear peptides) with the following sequence:

(SEQ ID NO: 74)
XP(R/X)(W/X)(D/X)APL(R/X)(D/F/X)PAL(R/X)(Q/X)
(L/X)(L/X),

where X represents any natural amino acid or amino acids specified in the below residue substitution tables 3-5. Certain preferred exemplified linear peptides have a sequence that is at least about 70% identical to that of a corresponding fragment of progranulin, or more preferably, as noted above, at least about 75% identical to such corresponding fragment (e.g., having a length within a range of about 4-20, preferably within a range of about 15-20, and in many cases 17 amino acids). In certain preferred exemplified peptides (and specifically in certain preferred exemplified linear peptides), one or more of positions P3, P4, P10, and P17 are modified (e.g., substituted with natural or non-natural amino acids relative to residues at those positions in human progranulin).

In the above formulas, X refers to all L-canonical amino acids or residues listed in the Substitution Tables (3-7) below, where positions are defined relative to the PGN sequence below (SEQ ID NO: 17):

TABLE 3
Basic residue analogues (L-Arginine in SEQ G23 at P3, SEQ D24 at P4, or P9/P14 in majority of peptides)

| P-6 | P-5 | P-4 | P-3 | P-2 | P-1 | P0 | P1 | P2 | P3 | P4 | P5 | P6 | P7 | P8 | P9 | P10 | P11 | P12 | P13 | P14 | P15 | P16 | P17 |
|---|---|---|---|---|---|---|---|---|---|---|---|---|---|---|---|---|---|---|---|---|---|---|---|
| T | K | C | L | R | R | E | A | P | R | W | D | A | P | L | R | D | P | A | L | R | Q | L | L |

Without wishing to be bound by any particular theory, we propose that basic amino acids may contribute to increasing affinity for SORT1 at this position; non-natural amino acid analogues may be particularly useful (e.g., may further enhance this structural feature in certain sortilin-binding peptides provided herein).

| Position | Name | SMILES |
|---|---|---|
| P3/P4 | D-Arginine | N[C@H](CCCNC(═N)N)C(═O)O |
| P3/P4 | L-Norarginine | N[C@@H](CCNC(═N)N)C(═O)O |
| P3/P4 | D-Norarginine | N[C@H](CCNC(═N)N)C(═O)O |
| P3/P4 | L-Citrulline | N[C@@H](CCCNC(═O)N)C(═O)O |
| P3/P4 | D-Citrulline | N[C@H](CCCNC(═O)N)C(═O)O |
| P3/P4 | L-Ornithine | N[C@@H](CCCN)C(═O)O |
| P3/P4 | D-Ornithine | N[C@H](CCCN)C(═O)O |
| P3/P4 | N5-(aminothioxomethyl)-D- | N[C@H](CCCNC(═S)N)C(═O)O |
| | Ornithine | |
| P3/P4 | N-Monomethyl-L-Arginine | N[C@@H](CCCNC(═N)NC)C(═O)O |
| P3/P4 | Beta-Homoarginine | N[C@@H](CCCNC(═N)N)CC(═O)O |
| P3/P4 | D-Beta-Homoarginine | N[C@H](CCCNC(═N)N)CC(═O)O |
| P3/P4 | NG,NG-Dimethyl-L-Arginine | N[C@@H](CCCNC(═N)N(C)C)C(═O)O |
| P3/P4 | N5-acetyl-L-ornithine | CC(═O)NCCC[C@@H](C(═O)O)N |
| P3/P4 | N2-Methyl-L-Arginine | N(C)[C@@H](CCCNC(═N)N)C(═O)O |
| P3/P4 | N2-Methyl-D-Arginine | N(C)[C@H](CCCNC(═N)N)C(═O)O |

Without wishing to be bound by any particular theory, we propose that hydrophobic amino acids may contribute to increases in affinity for SORT1 at this position; non-natural amino acid analogues may be particularly useful (e.g., may further enhance this structural feature in certain sortilin-binding peptides provided herein).

TABLE 4
Hydrophobic residue analogues (L-Tryptophan in SEQ G23 at P4)

| Position | Name | SMILES |
|---|---|---|
| P4 | D-Tryptophan | N[C@H](Cc1c[nH]c2c1cccc2)C(═O)O |
| P4 | N-methyl-L-Tryptophan | N(C)[C@@H](Cc1c[nH]c2c1cccc2)C(═O)O |
| P4 | N-methyl-D-Tryptophan | N(C)[C@H](Cc1c[nH]c2c1cccc2)C(═O)O |
| P4 | (4R)-4-amino-5-(1H- | C1═CC═C2C(═C1)C(═CN2)C[C@@H](CCC(═O)O)N |
| | indol-3-yl)pentanoic acid | |
| P4 | 5-Hydroxy-L-Tryptophan | N[C@@H](Cc1c[nH]c2c1cc(O)cc2)C(═O)O |
| P4 | 5-Fluoro-L-Tryptophan | N[C@@H](Cc1c[nH]c2c1cc(F)cc2)C(═O)O |
| P4 | 5-Methyl-L-Tryptophan | N[C@@H](Cc1c[nH]c2c1cc(C)cc2)C(═O)O |
| P4 | 6-Methyl-L-Tryptophan | N[C@@H](Cc1c[nH]c2c1ccc(c2)C)C(═O)O |
| P4 | 6-Hydroxy-L-Tryptophan | N[C@@H](Cc1c[nH]c2c1ccc(c2)O)C(═O)O |
| P4 | 5-Methyl-D-Tryptophan | N[C@H](Cc1c[nH]c2c1cc(cc2)C)C(═O)O |
| P4 | 6-Methyl-D-Tryptophan | N[C@H](Cc1c[nH]c2c1ccc(c2)C)C(═O)O |
| P4 | 5-Hydroxy-D-Tryptophan | N[C@H](Cc1c[nH]c2c1cc(O)cc2)C(═O)O |
| P4 | 6-Hydroxy-D-Tryptophan | N[C@H](Cc1c[nH]c2c1ccc(c2)O)C(═O)O |
| P4 | 5-Chloro-L-Tryptophan | N[C@@H](Cc1c[nH]c2ccc(Cl)cc12)C(═O)O |
| P4 | 6-Chloro-L-Tryptophan | N[C@@H](Cc1c[nH]c2cc(Cl)ccc12)C(═O)O |

TABLE 5
Long hydrophobic residue analogues
(L-Norleucine in SEQ G23 at P10)

| Position | Name | SMILES |
|---|---|---|
| P10 | D-Norleucine | N[C@H](CCCC)C(═O)O |
| P10 | (2S)-2-Aminoheptanoic acid | N[C@@H](CCCCC)C(═O)O |
| P10 | (R)-2-Aminoheptanoic acid | N[C@H](CCCCC)C(═O)O |
| P10 | (S)-2-Aminooctanoic acid | N[C@@H](CCCCCC)C(═O)O |
| P10 | (R)-2-Aminooctanoic acid | N[C@H](CCCCCC)C(═O)O |
| P10 | L-Norvaline | N[C@@H](CCC)C(═O)O |
| P10 | D-Norvaline | N[C@H](CCC)C(═O)O |
| P10 | L-Ornithine | N[C@@H](CCCN)C(═O)O |
| P10 | Pentahomo-L-Serine | N[C@@H](CCCO)C(═O)O |
| P10 | D-Pentahomoserine | N[C@H](CCCO)C(═O)O |
| P10 | D-Ornithine | N[C@H](CCCN)C(═O)O |
| P10 | L-Homomethionine | N[C@@H](CCCSC)C(═O)O |
| P10 | D-Homomethionine | N[C@H](CCCSC)C(═O)O |
| P10 | D-Methionine | N[C@H](CCSC)C(═O)O |
| P10 | L-Ethionine | ′N[C@@H](CCSCC)C(═O)O |
| P10 | D-Ethionine | N[C@H](CCSCC)C(═O)O |
| P10 | Hydroxy-L-Methionine | N[C@@H](CCSCO)C(═O)O |
| P10 | Hydroxy-D-Methionine | N[C@H](CCSCO)C(═O)O |
| P10 | L-2,4-Diaminobutanoic acid | N[C@@H](CCN)C(═O)O |
| P10 | (R)-2,4-Diaminobutanoic acid | N[C@H](CCN)C(═O)O |

Without wishing to be bound by any particular theory, the present disclosure provides an insight that hydrophobic amino acids may contribute to increases in affinity for SORT1 at this position; non-natural amino acid analogues may be particularly useful (e.g., may further enhance this structural feature in certain sortilin-binding peptides provided herein).

Without wishing to be bound by any particular theory, the present disclosure proposes that reactive amino acids may contribute to increased stability of SORT1 at this position; and non-natural amino acid analogues further enhance this structural feature of our peptide series.

TABLE 6
Disulfide-linkage residue analogues (L-Cysteine
in SEQ G23 at P5/P13, in P8/P12 in D24)

| Position | Name | SMILES |
|---|---|---|
| P5/P13/etc | D-Cysteine | N[C@H](CS)C(═O)O |
| P5/P13/etc | Homo-L-Cysteine | N[C@@H](CCS)C(═O)O |
| P5/P13/etc | Homo-D-Cysteine | N[C@H](CCS)C(═O)O |
| P5/P13/etc | L-Penicillamine | N[C@@H](C(S)(C)C)C(═O)O |
| P5/P13/etc | D-Penicillamine | N[C@H](C(C)(C)S)C(═O)O |

TABLE 7
P16/P17 Analogues (Leucine in SEQ G23 or D24 at P16 and P17)

| Position | Name | SMILES |
|---|---|---|
| P16/P17 | 3-Cyclopropyl-L- | N[C@@H](CC1CC1)C(═O)O |
| | Alanine (F01) | |
| P16/P17 | 4-Methyl-L-Leucine | N[C@@H](CC(C)(C)C)C(═O)O |
| | (F02) | |
| P16/P17 | Beta-cyclobutyl-L- | C1CC(C1)C[C@@H](C(═O)O)N |
| | alanine (G48) | |
| P16/P17 | L-Norleucine | N[C@@H](CCCC)C(═O)O |
| | (A45/Nle) | |
| P16/P17 | 3-Methyl-L-Valine | N[C@@H](C(C)(C)C)C(═O)O |
| | (B13) | |
| P16/P17 | Homo-L-Leucine | N[C@@H](CCC(C)C)C(═O)O |
| | (B43) | |
| P16/P17 | D-Leucine | N[C@H](CC(C)C)C(═O)O |
| P16/P17 | (2S,4R)-2-Amino-4- | N[C@@H](C[C@@H](C)O)C(═O)O |
| | hydroxypentanoic acid | |
| P16/P17 | (2R,4S)-2-Amino-4- | N[C@H](C[C@H](C)O)C(═O)O |
| | hydroxypentanoic acid | |
| P16/P17 | (4S)-4-Hydroxy-L- | C[C@@H](C[C@@H](C(═O)O)N)O |
| | Norvaline | |
| P16/P17 | (4R)-4-Hydroxy-D- | N[C@H](C[C@@H](C)O)C(═O)O |
| | Norvaline | |
| P16/P17 | 5-Methyl-D- | N[C@H](CCC(C)C)C(═O)O |
| | Norleucine | |

Without wishing to be bound by any particular theory, the present disclosure provides an insight that hydrophobic amino acids may contribute to increased affinity to SORT1 at this position, and/or that non-natural amino acid analogues may be particularly useful (e.g., may further enhance this structural feature in sortilin-binding peptides provided herein).

### Conjugation Sites

At least the following sites are identified as amenable for conjugation with the linker and the payload:
P3 for cyclic group 1
P4 for cyclic group 1 & 2
P8 for cyclic group 1
P10 for cyclic group 2

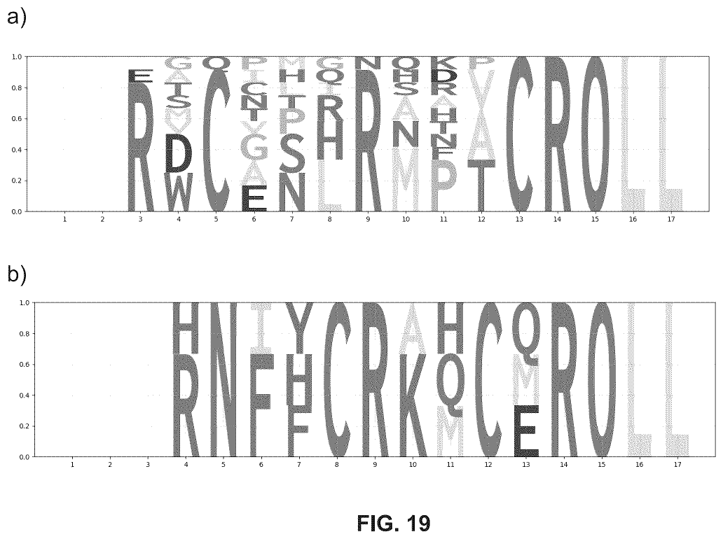

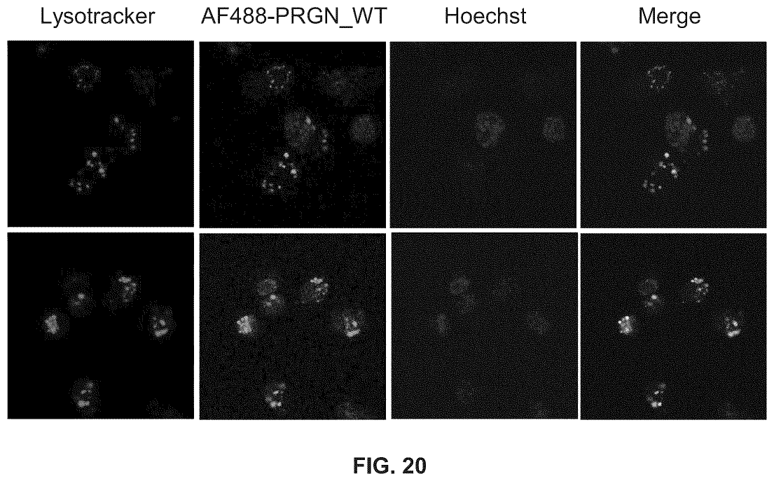

FIG. 19 presents exemplary Sequence Logos for these two cyclic groups.

Peptides that showed highest binding (higher i-density on the array) were selected and further synthesized in SPPS.

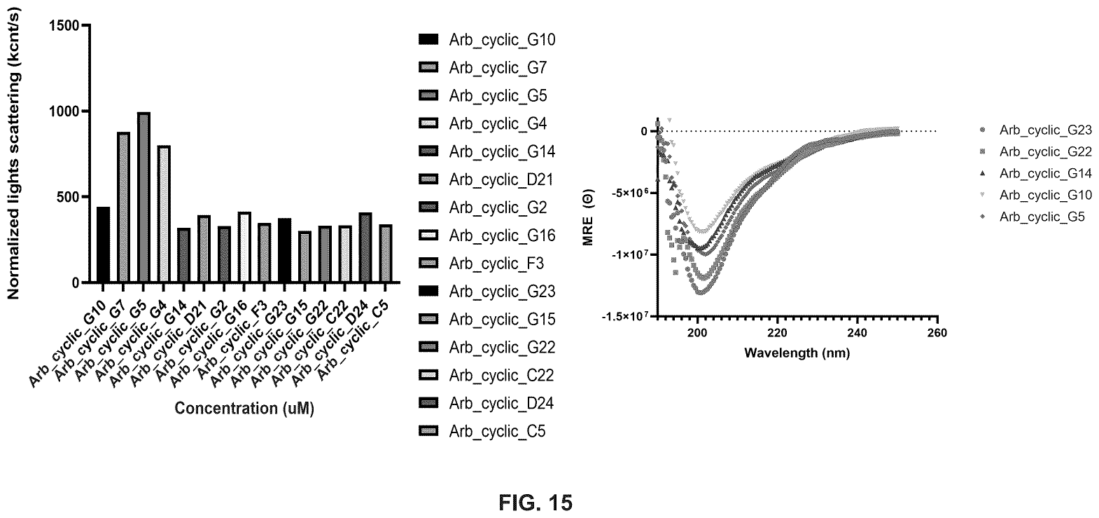

A total of 19 linear hits and 15 cyclic hits were characterized for their solubility in DLS and secondary structure in CD (Table 2). We found that 95% demonstrated good solubility at the working concentrations (250 nM, PBS, pH 7.4) and exhibited a random coil secondary structure. 3 of the linear designs had DLS results that suggested they were aggregating and so were excluded from the remaining workflow. (FIGS. 14-15).

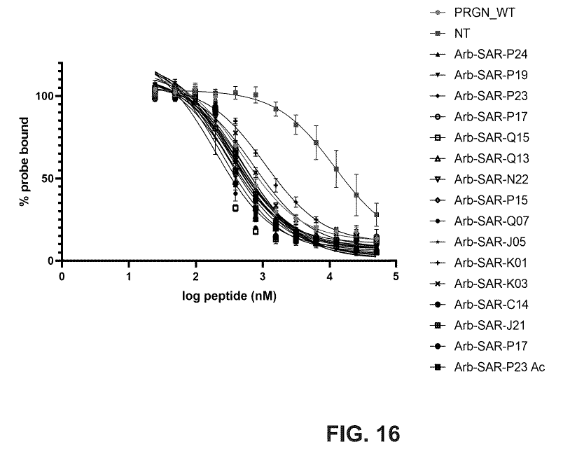

We profiled the 16 soluble linear array hits using the FP competition assay. Of these, 10 sequences demonstrated improved affinity for SORT1 compared to PRGN_WT (FIG. 16, Table 8). In particular, Arb-SAR-Q15 showed 2-fold higher affinity compared with PRGN_WT (IC50=233.35 nM).

TABLE 8
Table of IC50s. Binding of certain exemplary linear peptides to SORT1
(Table discloses SEQ ID NOS 29, 34, 75, 39-44, 30, 45-49, 23 and 50-54,
respectively, in order of appearance)

| | | | | | IC50 in a SORT1 |
|---|---|---|---|---|---|
| | | | | | FP competition |
| Index | Name | N-term | Sequence | C-term | assay, nM |
| 1 | PRGN_WT | NH2 | APRWDAPLRDPALRQLL | COOH | 452.1 |
| 2 | PRGN_RL | NH2 | DDPRAPWPALQRLALRL | COOH | 3076 |
| 3 | NT | NH2 | QLYENKPRRPYIL | COOH | 11890 |
| 4 | Arb-SAR-P21 | NH2 | APRWDAPLRDPALRQ(F01)(F02) | COOH | 969.5 |
| 5 | Arb-SAR-P24 | NH2 | APRWDAPLRDPALRQ(G48)(F02) | COOH | 382.9 |
| 6 | Arb-SAR-P22 | NH2 | APRWDAPLRDPALRQ(F02)(F02) | COOH | 324.5 |
| 7 | Arb-SAR-P19 | NH2 | APRWDAPLRDPALRQ(A45)(F62) | COOH | 625.2 |
| 8 | Arb-SAR-P23 | NH2 | APRWDAPLRDPALRQ(B13)(F82) | COOH | 522.75 |
| 9 | Arb-SAR-Q09 | NH2 | APRWDAPLRDPALRQ(B43)(648) | COOH | 346.5 |
| 10 | Arb-SAR-Q15 | NH2 | APRWDAPLRDPALRQ(B13)(G48) | COOH | 233.35 |
| 11 | Arb-SAR-Q13 | NH2 | APRWDAPLRDPALRQ(F81)(G48) | COOH | 370.6 |
| 12 | Arb-SAR-N22 | NH2 | APRWDAPLRDPALRQ(843)(843) | COOH | 397.45 |
| 13 | Arb-SAR-P15 | NH2 | APRWDAPLRDPALRQF(F82) | COOH | 456.75 |
| 14 | Arb-SAR-Q7 | NH2 | APRWDAPLRDPALRQF(G48) | COOH | 282.3 |
| 15 | Arb-SAR-J05 | NH2 | APRWDAPLRDPALRQL(F02) | COOH | 431.2 |
| 16 | Arb-SAR-K01 | NH2 | APRWDAPLRDPALR(843)LL | COOH | 1311 |
| 17 | Arb-SAR-K3 | NH2 | APRWDAPLRDPALR(850)LL | COOH | 682.45 |
| 18 | Arb-SAR-C14 | NH2 | APRWDAPLRDPALRQLL | COOH | 293.25 |
| 19 | Arb-SAR-J21 | NH2 | APRWDAPLRDPALRQ(F02)L | COOH | 344.8 |
| 20 | Arb-SAR-P17 | NH2 | APRWDAPLRDPALRQ(B43)(F02) | COOH | 479.6 |
| 21 | Arb-SAR-P23-Ac | Ac | APRWDAPLRDPALRQ(B13)(F62) | COOH | 534.9 |

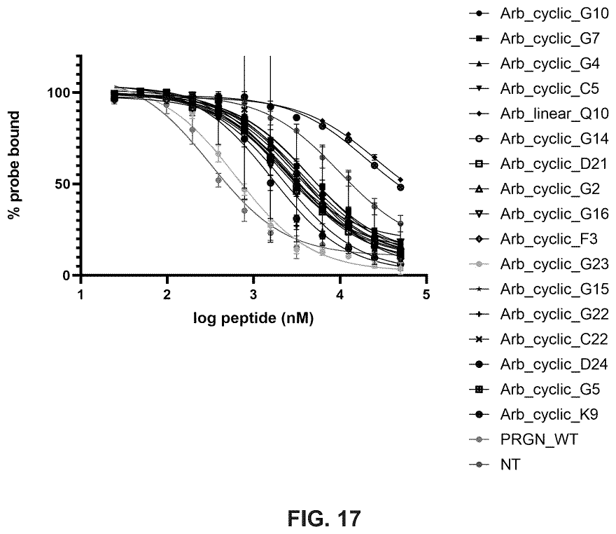

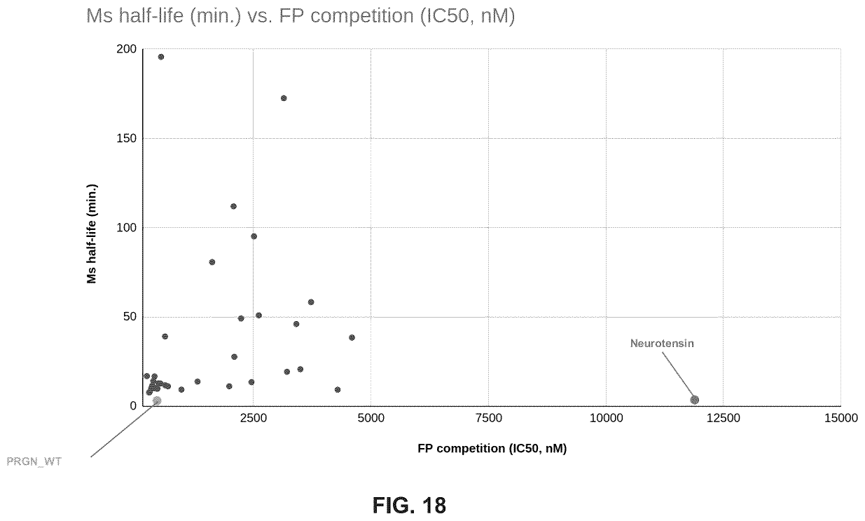

Out of 15 cyclic array hits, Arb_cyclic_G23 displayed comparable SORT1 affinity compared to PRGN_WT (FIG. 17, Table 9, IC50=620 nM). SORT1 affinity of the remaining cyclic designs range from 1621 nM-4592 nM.

The stability of the computationally designed peptides was further assessed in a murine serum stability assay (Tables 10-11). Out of 17 profiled linear sequences, 15 demonstrated more than 3-fold longer half-lives compared to PRGN_WT. In particular, Arb-SAR-Q15 possessed a T½ of 16.92 minutes. Cyclic peptides demonstrated significantly longer half-lives overall. All 15 profiled cyclic sequences demonstrated more than 3-fold longer half-lives compared to PRGN_WT, and 10 of them demonstrated more than 10-fold increase. Arb_cyclic_F3 and Arb_cyclic_G10 demonstrated particularly long half-lives of 172.58 and 112.02 minutes, respectively.

TABLE 9
Table of IC50s. Binding of certain exemplary cyclic peptides to SORT1 (Table
discloses SEQ ID NOS 55-69 and 75, respectively, in order of appearance)

| | | | | | IC50 in a SORT1 |
|---|---|---|---|---|---|
| | | | | | FP competition |
| Index | Name | N-term | Sequence | C-term | assay, nM |
| 1 | Arb_cyclic_G10 | Ac | R(Nle)CGPIRSPACRQ(B13)(F02) | COOH | 2077 |
| 2 | Arb_cyclic_G7 | AC | RGCIPRRQDACRQ(B13){F02) | COOH | 4288 |
| 3 | Arb_cyclic_G5 | Ac | RDCVSLR(Nle)HVCRQ(B13)(F82) | COOH | 2510 |
| 4 | Arb cyclic_G4 | AC | RDCPSLRAKVCRQ(B13)(F02) | COOH | 4592 |
| 5 | Arb_cyclic_G14 | AC | RSCTTHR(Nle)ATCRQ(B13)(F02) | COOH | 2612 |
| 6 | Arb cyclic D21 | AC | RNFHCRAQC(Nle)RQ(B13)(F02) | COOH | 2457 |
| 7 | Arb_cyclic_G2 | Ac | RDCASLRAPPCRQ(B13)(F02) | COOH | 3496 |
| 8 | Arb_cyclic_G16 | AC | RYCANHR(Nle)NTCRQ(B13)(F02) | COOH | 1985 |
| 9 | Arb_cyclic_F3 | AC | EWCGLHNHPTCRQ(B13)(F02) | COOH | 3142 |
| 10 | Arb_cyclic_G23 | Ac | RWCENRR(Nle)FTCRQ(B13)(F02) | COOH | 620 |
| 11 | Arb_cyclic_G15 | Ac | RTCNHERNPACRQ(B13)(F02) | COOH | 3212 |
| 12 | Arb_cyclic_G22 | Ac | RWCENQR(Nle)TVCRQ(B13)(F02) | COOH | 2235 |
| 13 | Arb cyclic C22 | AC | HNFYCRKHCQRQ(B13)(F02) | COOH | 3722 |
| 14 | Arb_cyclic_D24 | Ac | RNIFCEK(Nle)CERQ(B13)(F02) | COOH | 1621 |
| 15 | Arb_cyclic_C5 | Ac | AQC(NLe)GRNRACRQ(B13)(F82) | COOH | 3410 |
| 16 | NT | NH2 | QLYENKPRRPYIL | COOH | 8339 |

TABLE 10
Table of serum stability. Serum stability of certain exemplary linear peptides
(Table discloses SEQ ID NOS 29, 34, 39, 76, 41-44, 30, 45-49, 23 and 50-53, respectively,
in order of appearance)

| | | | | | | | Murine serum |
|---|---|---|---|---|---|---|---|
| | | | | | | | stability assay, |
| Index | Name | N-term | Sequence | C-term | Cyclized | Type | T1/2 (min) |
| 1 | PRGN_WT | NH2 | APRWDAPLRDPALRQLL | COOH | linear | homing peptide | 3.16 |
| 2 | PRGN_RL | NH2 | DDPRAPWPALQRLALRL | COOH | linear | homing peptide | 27.7 |
| 9 | Arb-SAR-P21 | NH2 | APRWDAPLRDPALRQ(F01)(F02) | COOH | linear | homing peptide | 9.33 |
| 10 | Arb-SAR-P24 | NH2 | APRWDAPLRDPALRQ(G48)(F02) | COOH | linear | homing peptide | 10.08 |
| 11 | Arb-SAR-P22 | NH2 | APRWDAPLRDPALRQ(F02){F02} | COOH | linear | homing peptide | 9.96 |
| 12 | Art-SAR-P18 | NH2 | APRWDAPLRDPALRQ(A45}{F02) | COOH | linear | homing peptide | 11.75 |
| 13 | Aro-SAR-F23 | NH2 | APRWDAPLRDPALRQ(B13){F02) | COOH | linear | homing peptide | 12.73 |
| 14 | Arb-SAR-Q09 | NH2 | APRWDAPLRDPALRQ(B43)(G48) | COOH | linear | homing peptide | 11.45 |
| 15 | Aro-SAR-Q15 | NH2 | APRWDAPLRDPALRQ(B13)(G48) | COOH | linear | homing peptide | 18.92 |
| 16 | Arb-SAR-Q13 | NH2 | APRWDAPLRDPALRQ(F01)(G48) | COOH | linear | homing peptide | 14.11 |
| 17 | Arb-SAR-N22 | NH2 | APRWDAPLRDPALRQ(B43)(843) | COOH | linear | homing peptide | 16.72 |
| 18 | Arb-SAR-P15 | NH2 | APRWDAPLRDPALRQF(F02) | COOH | linear | homing peptide | 9.79 |
| 19 | Arb-SAR-Q7 | NH2 | APRWDAPLRDPALRQF(G48) | COOH | linear | homing peptide | 7.73 |
| 20 | Aro-SAR-J05 | NH2 | APRWDAPLRDPALRQL(F02) | COOH | linear | homing peptide | 10.13 |
| 21 | Arb-SAR-K01 | NH2 | APRWDAPLRDPALR{B43)LE | COOH | linear | homing peptide | 13.81 |
| 22 | Arb-SAR-K3 | NH2 | APRWDAPLRDPALR(B50)LE | COOH | linear | homing peptide | 11.17 |
| 23 | Arb-SAR-C14 | NH2 | APRWDAPLRDPALRQLL | COOH | linear | homing peptide | 7.91 |
| 24 | Arp-SAR-J21 | NH2 | APRWDAPLRDPALRQ(F02)L | COOH | linear | homing peptide | 11.02 |
| 25 | Arb-SAR-P17 | NH2 | APRWDAPLRDPALRQ(B43)(F02) | COOH | linear | homing peptide | 12.81 |

TABLE 11
Table of serum stability. Serum stability of computationally designed cyclic
peptides to SORT1 (Table discloses SEQ ID NOS 55-69, respectively, in order of
appearance.

| | | | | | | | Murine |
|---|---|---|---|---|---|---|---|
| | | | | | | | serum |
| | | | | | | | stability |
| | | | | | | | assay, |
| Index | Name | N-term | Sequence | C-term | Cyclized | Type | T1/2 (min) |
| 1 | Arb_cyclic_G10 | Ac | R(Nle)CGPIRSPACRQ(B13)(F02) | COOH | cyclized | homing peptide | 112.02 |
| 2 | Arb_cyclic_G7 | Ac | RGCIPRRQDACRQ(B13)(F02) | COOH | cyclized | homing peptide | 9.26 |
| 3 | Arb_cyclic_G5 | Ac | RDCVSLR(Nle)HVCRQ(B13)(F02) | COOH | cyclized | homing peptide | 95.2 |
| 4 | Arb_cyclic_G4 | Ac | RDCPSLRAKVCRQ(B13)(F02) | COOH | cyclized | homing peptide | 38.47 |
| 5 | Arb_cyclic_G14 | Ac | RSCTTHR(Nle)ATCRQ(B13)(F02) | COOH | cyclized | homing peptide | 50.94 |
| 6 | Arb_cyclic_D21 | Ac | RNFHGRAQC(Nle)RQ(B13)(F02) | COOH | cyclized | homing peptide | 13.49 |
| 7 | Arb_cyclic_G2 | Ac | RDCASLRAPPCRQ(B13)(F02) | COOH | cyclized | homing peptide | 20.75 |
| 8 | Arb_cyclic_G16 | Ac | RVCANHR(Nle)NTCRQ(B13)(F02) | COOH | cyclized | homing peptide | 11.19 |
| 9 | Arb_cyclic_F3 | Ac | EWCGLANHPTCRQ(B13)(F02) | COOH | cyclized | homing peptide | 172.58 |
| 10 | Arb_cyclic_G23 | Ac | RWCENRR(Nie)FTCRQ(818)(F92) | COOH | cyclized | homing peptide | 38.1 |
| 11 | Arb_cyclic_G15 | Ac | RTCNHLRNPACRQ(B13)(F02) | COOH | cyclized | homing peptide | 19.32 |
| 12 | Arb_cyclic_G22 | Ac | RWCENQR(Nie)TVCRQ(B13)(F82) | COOH | cyclized | homing peptide | 49.18 |
| 13 | Arb_cyclic_C22 | Ac | HNFYCRKHCQRQ(B13)(F92) | COOH | cyclized | homing peptide | 58.83 |
| 14 | Arb_cyclic_D24 | Ac | RNIFCRK(Nle)CERQ(B13)(F02) | COOH | cyclized | homing peptide | 80.74 |
| 15 | Arb_cyclic_C5 | Ac | AQC(Nle)GRNRACRQ(B13)(F02) | COOH | cyclized | homing peptide | 46.09 |

### Example 8—PRGN Based Non-Natural and Cyclized PDCs Tested in a Murine Xenograft Study

The present Example demonstrates effects of certain provided PRGN_based PDCs in a MDA-MB-231 xenograft efficacy model. Each of PRGN_WT_PDC, Arb-SAR-Q15-PDC, Arb_cyclic_G23-PDC, and a control PRGN_scr_PDC was dissolved in 25 mM Histidine 10% sucrose pH 7 and administered to mice at 3 mg/kg, i.v. at a QW×4 schedule.

To characterize the effects of the MMAE payload alone, we included free MMAE at molar equivalency to PDCs, and tested at 0.6 mg/kg.

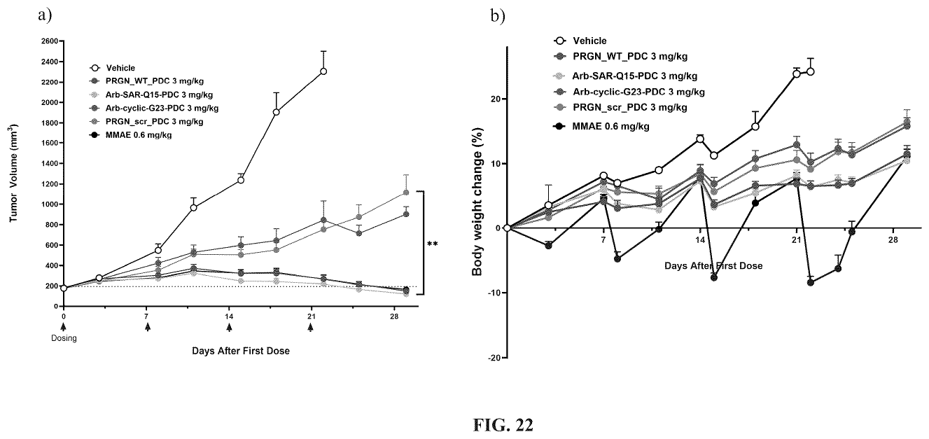

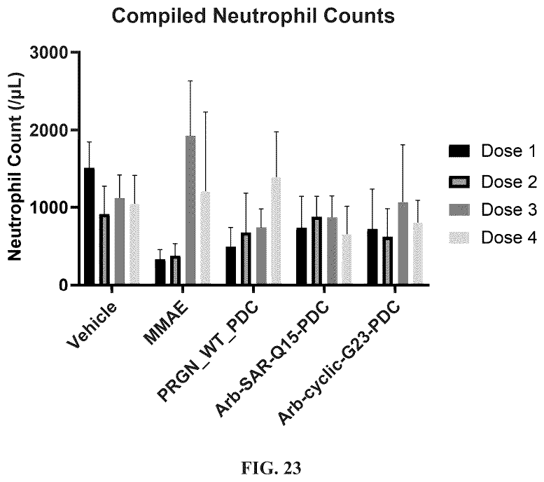

In the xenograft efficacy model, we observed tumor growth inhibition with PRGN_WT_PDC, Arb-SAR-Q15-PDC, Arb_cyclic_G23-PDC, and PRGN_scr_PDC (FIG. 22a, P≤0.01 by Two-way Anova). At 3 mg/kg treatment of PRGN_WT_PDC, Arb-SAR-Q15-PDC, Arb_cyclic_G23-PDC, and PRGN_scr_PDC, tumor growth was inhibited by 96%, 98%, 68%, and 73% respectively relative to vehicle at day 22 (FIG. 22a). It is hypothesized that PRGN_scr_PDC likely shows lower tumor inhibition because it has a lower affinity for the target.

We sought to evaluate the toxicity of PRGN_WT_PDC, Arb-SAR-Q15-PDC, Arb_cyclic_G23-PDC, and PRGN_scr_PDC relative to free MMAE by comparing body weight loss in the MDA-MB-231 xenograft model. For each of PRGN_WT_PDC, Arb-SAR-Q15-PDC, Arb_cyclic_G23-PDC, and PRGN_scr_PDC treatments, body weight loss tracked each other (FIG. 22b). Free MMAE administered at molar equivalency to PDCs resulted in body weight fluctuations corresponding to treatment times, while vehicle treatment resulted in the least loss of weight.

Without wishing to be bound by theory, we propose that the low toxicity observed with PRGN_WT_PDC, Arb-SAR-Q15-PDC, and Arb_cyclic_G23-PDC may result from specific targeting of SORT1-expressing MDA-MB-231 in the xenograft model, whereas the body weight fluctuations caused by the equivalent dose of MMAE may result from non-specific targeting of the free drug. Additionally, we propose that the low toxicity observed with PRGN_scr_PDC may result from conjugation of the scrambled (scr) peptide to various components to form a PDC.

### Pharmacokinetic Assessment

The kinetic rate constants (kₐ and k_{d}) and the equilibrium binding constant (K_{d}) for each of PRGN_WT_PDC, Arb-SAR-Q15-PDC, Arb_cyclic_G23-PDC was evaluated using SPR as detailed in Example 1. Table 12 tabulates the binding constants for each of the three PDCs tested.

TABLE 12
Table of Ka values. Binding of certain exemplary peptide drug conjugates to
SORT1 (Table discloses SEQ ID NOS 77-79, respectively, in order of appearance)

| Index | Name | N-term | Sequence | C-term | K in SPR assay (nM) |
|---|---|---|---|---|---|
| 1 | PRGN_WT_ | VCPAB-MMAE | bAla-SarsarSarsar- | COOH | 0.782 |
| | PDC | | APRWDAPLRDPALRQLL | | |
| 2 | Arb-SAR- | VCPAR-MMAE | bAla-SarSarSerSar- | COOH | 0.387 |
| | Q15-PDC | | APRWDAPLRDPALRQ | | |
| | | | (b13)(G48) | | |
| 3 | Arb-cyclic- | Ac | RWCENK(SarSerSarSar- | COOH | 0.890 |
| | G23-PDC | | bAla-VCPAB-MMAE)R(Nle) | | |
| | | | FTCRQ(B13)(F02) | | |

### Murine Plasma Stability Assay

The plasma stability for each of PRGN_WT_PDC, Arb-SAR-Q15-PDC, Arb_cyclic_G23-PDC was evaluated using the murine plasma stability assay as detailed in Example 1. Table 13 tabulates the plasma stability for each of the three PDCs tested.

TABLE 13
Table of plasma stability. Plasma stability of
certain exemplary peptide drug conjugates to SORT1
(Table discloses SEQ ID NOS 77-79,
respectively, in order of appearance)

| | | | | | Murine |
|---|---|---|---|---|---|
| | | | | | plasma |
| | | | | | stability |
| | | | | | assay, |
| | | | | | T1/2 |
| Index | Name | N-term | Sequence | C-term | (min) |
| 1 | PRON_WT_ | VCPAB- | bAla-SarsarSarsar- | COOH | 17.1 |
| | PDC | MMAE | APRWDAPLRDPALRQLL | | |
| 2 | Arb-SAR- | VCPAB- | bAla-SarSarSerSar- | OOOH | 49.7 |
| | Q15-PDC | MMAE | APRWDAPLRDPALRQ | | |
| | | | (b13)(G48) | | |
| 8 | Arb-cyclic- | | RWCENK(SarSerSarSar- | COOH | 57.4 |
| | G23-PDC | | bAla-VCPAB-MMAE)R | | |
| | | | (Nle)FTCRQ(B13)(F02) | | |

As shown in Table 13, T12 for PGRN_WT_PDC was determined to be 17.1 minutes, which is significantly lower than the other two PDCs tested, i.e., Arb-SAR-Q15-PDC (T12=49.7 minutes) and Arb_cyclic_G23-PDC (T1 2=57.4 minutes). Despite the lower serum half-life of PRGN_WT_PDC compared to other PDCs tested, PRGN_WT_PDC treatment resulted in significant tumor growth inhibition. For example, both PRGN_WT_PDC and Arb-SAR-Q15-PDC treatment surprisingly resulted in almost similar tumor growth inhibition of 96% and 98%, respectively in a murine xenograft efficacy model. Additionally, PRGN_WT_PDC treatment surprisingly resulted in significantly increased tumor growth inhibition of 96% compared to 68% inhibition by Arb_cyclic_G23-PDC treatment (see FIG. 22a).

That is, the inventors of the present application surprisingly found that while in-vitro PGRN_WT_PDC may not appear to be as effective in targeting and/or reducing tumor growth due to its lower half-life compared to other PDCs like Arb-SAR-Q15-PDC and Arb_cyclic_G23-PDC, in-vivo murine models show that PGRN_WT_PDC surprising performed just as well, if not significantly better than PDCs with higher half-lives.

That is, in some embodiments, a PDC with less in-vitro stability may be used in methods of treatment or compositions as described herein to achieve comparable or better therapeutic results (e.g., tumor growth inhibition).

### Murine Hematotoxicity Assay

As outlined in the material and methods of Example 1, wild type BALB/c, female, 7-8 weeks, weighing approximately 18-22 g were randomized and dosed intravenously with control or test articles at a QW×4 schedule. Dosing volume was adjusted based on body weight and was administered at 10 ml/Kg. PRGN_WT_PDC, Arb-SAR-Q15-PDC, and Arb_cyclic_G23-PDC were dissolved in 25 mM Histidine 10% sucrose pH 7 and 3 mg/kg was administered. Free MMAE payload was also tested, and similarly dissolved in 25 mM Histidine 10% sucrose pH 7 and administered to the mice at a dose of 0.6 m/kg by i.v. injection at a QW×4 schedule. 3 days following each dose, 150 μL of whole blood was collected via submandibular method and placed in K2 EDTA tubes stored on ice. Blood neutrophil counts were performed on the day of collection by IDEXX labs. Body weights were recorded twice a week until study endpoint.

Results of this study are shown in FIG. 24. As demonstrated, PRGN_WT_PDC, Arb-SAR-Q15-PDC, and Arb_cyclic_G23-PDC show reduced neutrophil counts compared to vehicle or MMAE treatment.

### REFERENCES

1. Petersen, C. M., Nielsen, M. S., Jacobsen, C., Tauris, J., Jacobsen, L., Gliemann, J., Moestrup, S. K. & Madsen, P. (1999). Propeptide cleavage conditions sortilin/neurotensin receptor-3 for ligand binding. The EMBO Journal, 18(3), 595-604.
2. Bartkowska, K., Turlejski, K. & Djavadian, R. L. (2010). Neurotrophins and their receptors in early development of the mammalian nervous system. Acta Neurobiologiae Experimentalis, 70(4), 454-467.
3. Zhou, X., Paushter, D. H., Feng, T., Sun, L., Reinheckel, T. & Hu, F. (2017). Lysosomal processing of progranulin. Molecular Neurodegeneration, 12(1), 62.
4. Nielsen, M. S., Jacobsen, C., Olivecrona, G., Gliemann, J. & Petersen, C. M. (1999). Sortilin/Neurotensin Receptor-3 Binds and Mediates Degradation of Lipoprotein Lipase*. Journal of Biological Chemistry, 274(13), 8832-8836.
5. Kim, J. T., Napier, D. L., Weiss, H. L., Lee, E. Y., Townsend, C. M. & Evers, B. M. (2017). Neurotensin Receptor 3/Sortilin Contributes to Tumorigenesis of Neuroendocrine Tumors Through Augmentation of Cell Adhesion and Migration1. Neoplasia (New York, N.Y.), 20(2), 175-181.
6. Rhost, S., Hughes, É., Harrison, H., Rafnsdottir, S., Jacobsson, H., Gregersson, P., Magnusson, Y., Fitzpatrick, P., Andersson, D., Berger, K., Ståhlberg, A. & Landberg, G. (2018). Sortilin inhibition limits secretion-induced progranulin-dependent breast cancer progression and cancer stem cell expansion. Breast Cancer Research: BCR, 20(1), 137.
7. Roselli, S., Pundavela, J., Demont, Y., Faulkner, S., Keene, S., Attia, J., Jiang, C. C., Zhang, X. D., Walker, M. M. & Hondermarck, H. (2015). Sortilin is associated with breast cancer aggressiveness and contributes to tumor cell adhesion and invasion. Oncotarget, 6(12), 10473-10486.
8. Berger, K., Rhost, S., Rafnsdóttir, S., Hughes, É., Magnusson, Y., Ekholm, M., Stål, O., Rydén, L. & Landberg, G. (2021). Tumor co-expression of progranulin and sortilin as a prognostic biomarker in breast cancer. BMC Cancer, 21(1), 185.
9. Gao, F., Griffin, N., Faulkner, S., Li, X., King, S. J., Jobling, P., Denham, J. W., Jiang, C. C. & Hondermarck, H. (2020). The Membrane Protein Sortilin Can Be Targeted to Inhibit Pancreatic Cancer Cell Invasion. The American Journal of Pathology, 190(9), 1931-1942.
10. Akil, H., Perraud, A., Mélin, C., Jauberteau, M.- O. & Mathonnet, M. (2011). Fine-Tuning Roles of Endogenous Brain-Derived Neurotrophic Factor, TrkB and Sortilin in Colorectal Cancer Cell Survival. PloS ONE, 6(9), e25097.
11. Ghaemimanesh, F. (n.d.). The Effect of Sortilin Silencing on Ovarian Carcinoma Cells.
12. Hemmati, S., Zarnani, A. H., Mahmoudi, A. R., Sadeghi, M.- R., Soltanghoraee, H., Akhondi, M. M., Tarahomi, M., Jeddi-Tehrani, M. & Rabbani, H. (2009). Ectopic Expression of Sortilin 1 (NTR-3) in Patients with Ovarian Carcinoma. Avicenna Journal of Medical Biotechnology, 1(2), 125-131.
13. Farahi, L., Ghaemimanesh, F., Milani, S., Razavi, S. M., Akhondi, M. M. & Rabbani, H. (2019). Sortilin as a Novel Diagnostic and Therapeutic Biomarker in Chronic Lymphocytic Leukemia. Avicenna Journal of Medical Biotechnology, 11(4), 270-276.
14. Zheng, Y., Brady, O. A., Meng, P. S., Mao, Y. & Hu, F. (2011). C-Terminus of Progranulin Interacts with the Beta-Propeller Region of Sortilin to Regulate Progranulin Trafficking. PloS ONE, 6(6), e21023.
15. Hu, F., Padukkavidana, T., Vægter, C. B., Brady, O. A., Zheng, Y., Mackenzie, I. R., Feldman, H. H., Nykjaer, A. & Strittmatter, S. M. (2010). Sortilin-Mediated Endocytosis Determines Levels of the Frontotemporal Dementia Protein, Progranulin. Neuron, 68(4), 654-667.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A conjugate comprising:
(a) polypeptide; and
(b) a payload; and
(c) optionally, a linker,
wherein the polypeptide includes a sortilin binding moiety that:
has a length within a range of about 12 to about 20 amino acids and includes a characteristic sequence represented by:
(SEQ ID NO: 1)
(R/N) X²⁻³ (C/L) X⁰⁻¹ R (Q/E/L/B43/B50)
or
(SEQ ID NO: 2)
A P R W D A P L R X P A L R;
wherein X is any canonical or non-canonical amino acid.

2. The conjugate of claim 1, wherein the sortilin binding moiety:
corresponds to a C-terminal fragment of progranulin, or a variant thereof, and includes not more than about 20 contiguous residues corresponding to contiguous progranulin residues.

3. The conjugate of claim 1, wherein the sortilin binding moiety is or comprises a cyclic peptide.

4. The conjugate of claim 1, wherein the characteristic sequence is represented by:
(SEQ ID NO: 3)
(R/N) X²⁻³ C X⁰⁻¹ R (Q/E)

5. The conjugate of claim 1, wherein the characteristic sequence is represented by:
(SEQ ID NO: 4)
(R/N) X²⁻³ L X⁰⁻¹ R (Q/B43/B50)

6. The conjugate of claim 1, wherein the sortilin binding moiety is or comprises a linear peptide and the characteristic sequence is represented by SEQ ID NO: 2.

7. The conjugate of claim 5, wherein the sortilin binding moiety is or comprises a linear peptide.

8. (canceled)

9. The conjugate of claim 1, wherein the characteristic sequence is represented by:
(SEQ ID NO: 5)
(R/N) X²⁻³ C/L X⁰⁻¹ R Q (L/B13/F02) (L/B13/F02).

10. The conjugate of claim 1, wherein the characteristic sequence is represented by:
(SEQ ID NO: 6)
(R/N) X²⁻³ C X⁰⁻¹ R Q (L/B13/F02) (L/B13/F02).

11. The conjugate of claim 1, wherein the characteristic sequence includes at least a second cysteine residue and the polypeptide includes at least one disulfide bond between cysteine residues.

12. The conjugate of claim 1, wherein the characteristic sequence is represented by:
(SEQ ID NO: 7)
(R/N) X10 X11 X12 C R Q.

13. The conjugate of claim 1, wherein the characteristic sequence is represented by:
(SEQ ID NO: 8)
X4 X5 X6 X7 X8 (R/N) X10 X11 X12 C R Q;
wherein at least one of X4, X5, and X6 is a cysteine residue, wherein the polypeptide includes at least one disulfide bond between cysteine residues.

14. (canceled)

15. The conjugate of claim 1, wherein the characteristic sequence is represented by:
(SEQ ID NO: 9)
(R/E) X4 X5 X6 X7 X8 (R/N) X10 X11 X12 C R Q;
wherein at least one of X4, X5, and X6 is a cysteine residue, wherein the polypeptide includes at least one disulfide bond between cysteine residues.

16. (canceled)

17. The conjugate of claim 1, wherein the characteristic sequence is represented by:
(SEQ ID NO: 10)
(R/E) X4 X5 X6 X7 X8 (R/N) X10 X11 X12 C R Q
(L/B13/F02) (L/B13/F02);
wherein at least one of X4, X5, and X6 is a cysteine residue, wherein the polypeptide includes at least one disulfide bond between cysteine residues.

18. (canceled)

19. The conjugate of claim 1, wherein the characteristic sequence is represented by:
(SEQ ID NO: 11)
C R X10 X11 C X13 R Q;
wherein the two cysteine residues form a disulfide bond and the polypeptide is a cyclic polypeptide.

20. The conjugate of claim 1, wherein the characteristic sequence is represented by:
(SEQ ID NO: 12)
(R/H) N X6 X7 C R X10 X11 C X13 R Q;
wherein the two cysteine residues form a disulfide bond and the polypeptide is a cyclic polypeptide.

21. The conjugate of claim 1, wherein the characteristic sequence is represented by:
(R/H) N X6 X7 CR X10 X11 C X13 R Q (L/B13/F02) (L/B13/F02) (SEQ ID NO: 13);
wherein the two cysteine residues form a disulfide bond and the polypeptide is a cyclic polypeptide.

22. The conjugate of claim 1, wherein the sortilin binding moiety includes one or more of:
(i) a basic residue such as L-arginine or an analog thereof at a position corresponding to position P3 and/or P4 of a 17mer C-terminal fragment of progranulin;
(ii) a hydrophobic residue such as a L-tryptophan or an analog thereof at a position corresponding to position P4 of a 17mer C-terminal fragment of progranulin;
(iii) a long hydrophobic residue at a position corresponding to position P10 a 17mer C-terminal fragment of progranulin;
(iv) a covalent bond such as a disulfide bond) between residues at positions corresponding to positions P5 and P13 or P8 and P12 of a 17mer C-terminal fragment of progranulin; and
(v) a hydrophobic residue such as Leucine or a Leucine at a position corresponding to position P16 and/or P17 of a 17mer C-terminal fragment of progranulin.

23.-25. (canceled)

26. The conjugate of claim 1, wherein the payload is or comprises a therapeutic payload.

27. The conjugate of claim 1, wherein the sortilin binding moiety is characterized in that:
(i) it demonstrates affinity (Kd) for human sortilin 1 below about 1 μM when assessed by fluorescence polarization;
(ii) in a competitive binding assay with a reference C-terminal progranulin fragment, it demonstrates an IC₅₀ less than about 12 μM.

28. The conjugate of claim 27, wherein the sortilin binding moiety is further characterized in that, when maintained under murine serum, it displays stability greater than that of the reference C-terminal progranulin fragment.

29. The conjugate of claim 27, wherein the sortilin binding moiety is further characterized in that, when maintained under murine serum, it displays stability lower than that of the reference C-terminal progranulin fragment.

30. The conjugate of claim 27, wherein the reference C-terminal progranulin fragment has an amino acid sequence that is or comprises APRWDAPLRDPALRQLL.

31. The conjugate of claim 27, wherein the sortilin binding moiety is characterized by a stability half-life in murine serum that is greater than about >3 minutes.

32.-34. (canceled)

35. The conjugate of claim 30, characterized by an IC50 in the competitive binding assay that is less than about 5000 nM.

36.-41. (canceled)

42. The conjugate claim 35, wherein the sortilin binding moiety is or comprises a cyclic peptide.

43.-47. (canceled)

48. The conjugate of claim 35, wherein the sortilin binding moiety is or comprises a linear peptide.

49. The conjugate of claim 1, wherein the conjugate selectively or specifically targets sortilin-expressing cells.

50. The conjugate of claim 1, wherein the conjugate selectively or specifically targets cancer cells over normal cells.

51. The conjugate of claim 50, wherein the payload is or comprises a cytotoxic moiety and the conjugate kills the sortilin-expressing cells.

52. The conjugate of claim 51, wherein the conjugate kills the sortilin expressing cells with a potency greater than that observed for an otherwise identical conjugate whose sortilin binding moiety is or comprises a reference peptide that is a C-terminal fragment of progranulin.

53. The conjugate of claim 52, wherein the reference peptide has amino acid sequence APRWDAPLRDPALRQLL.

54. The conjugate of claim 53 wherein the potency is at least about 5 fold greater.

55. The conjugate of claim 50, wherein the cancer cells express sortilin at a level equivalent or above that at which sortilin is expressed by otherwise comparable noncancerous cells.

56. The conjugate of claim 1, wherein the conjugate undergoes cellular internalization.

57. The conjugate of claim 1, characterized in that the conjugate demonstrates improved affinity for sortilin relative to that observed with the unconjugated sortilin-binding moiety, wherein such improved affinity is at least about 2 fold greater.

58. (canceled)

59. The conjugate of claim 1, wherein the payload is or comprises a therapeutic or diagnostic moiety.

60. The conjugate of claim 1, wherein the payload is or comprises a therapeutic moiety.

61.-62. (canceled)

63. The conjugate of claim 51, wherein the cytotoxic moiety is characterized by a subnanomolar IC50.

64. The conjugate of claim 51, wherein the cytotoxic moiety is or comprises a bacterial toxin.

65. The conjugate of claim 1, wherein the payload is or comprises an anti-cancer agent.

66. The conjugate of claim 1, wherein the payload is selected from the group consisting of alkylating agents, anti-metabolites, anti-tumor antibiotics, boron neutron capture therapy agents, cell cycle inhibitors, kinesin spindle protein inhibitors, microtubule-binding agents, topoisomerase inhibitors, and combinations thereof.

67. The conjugate of claim 1, wherein the payload is or comprises an alkaloid, anthracycline, an auristatin, camptothecin, a folate derivative, a metal complex, a nucleoside analog, a taxane, a vinca alkaloid analog, or a combination thereof.

68. The conjugate of claim 1, wherein the payload is or comprises a phytochemical.

69. The conjugate of claim 1, wherein the payload is Monomethyl auristatin E (MMAE).

70. The conjugate of claim 1, wherein the payload is a detectable entity.

71. The conjugate of claim 1, wherein the payload is a small molecule.

72. The conjugate of claim 1, wherein the payload is a polypeptide.

73. The conjugate of claim 1, wherein the payload is an oligonucleotide.

74. The conjugate of claim 73, wherein the polypeptide oligonucleotide is an mRNA.

75. The conjugate of claim 1, wherein the payload is a particle.

76. The conjugate of claim 75, wherein the payload is a lipid nanoparticle.

77. The conjugate of claim 1, wherein the payload is a viral capsid.

78. The conjugate of claim 1, wherein the payload is a lipid vesicle such as an exosome or liposome.

79. The conjugate of claim 1, wherein the payload is or comprises a radioisotope.

80. The conjugate of claim 1, wherein the payload is covalently linked to the linker or the peptide by way of a hydroxyl, carboxyl, or amine group.

81. The conjugate of claim 1, wherein the payload is or comprises a protein degradation modulator such as a proteasome inhibitor or a PROTAC agent.

82. The conjugate of claim 1, wherein the payload is or comprises a nucleic acid editing system such as a CRISPR/Cas, a TALEN, an ADAR, etc.

83. The conjugate of claim 12, wherein the payload is connected to the amino acid residue in position P4.

84. The conjugate of claim 12, wherein the payload is connected to the amino acid residue in position P3.

85. The conjugate of claim 12, wherein the payload is connected to the amino acid residue in position P8.

86. The conjugate of claim 19, wherein the payload is connected to the amino acid residue in position P10.

87. The conjugate of claim 1, wherein at least one amino acid residue within the sequence of sortilin binding moiety is conjugated with a payload moiety.

88. The conjugate of claim 1, wherein at least one amino acid residue within the sequence outside of P15, P16 and P17 of sortilin binding moiety is conjugated with a payload moiety.

89. The conjugate of claim 1, wherein two or more amino acid residues within the sequence of sortilin binding moiety are conjugated with a payload moiety.

90. The conjugate of claim 1, wherein two or more amino acid residues within the sequence outside of P15, P16 and P17 of sortilin binding moiety is conjugated with a payload moiety.

91. The conjugate of claim 1, wherein the linker is cleavable.

92. The conjugate of claim 91, wherein the linker is an acid cleavable linker.

93. The conjugate of claim 91, wherein the linker is an enzyme cleavable linker.

94. The conjugate of claim 1, wherein the linker is a VCPAB linker.

95. The conjugate of claim 1, wherein the linker is or comprises an ester, an amide, a hydrazone, a carbonate, a reducible disulfide, and combinations thereof.

96. The conjugate of claim 1, wherein the linker is or comprises a thioether, an oxime, a triazole, and combinations thereof.

97. The conjugate of claim 1, wherein the linker is redox-sensitive.

98.-127. (canceled)

128. A pharmaceutical composition that comprises or delivers a conjugate of claim 1.

129. The pharmaceutical composition of claim 128, which comprises:
an active agent that is or comprises the conjugate; and
at least one pharmaceutically acceptable carrier.

130. The pharmaceutical composition of claim 129, wherein the payload is an anti-cancer agent.

131. The pharmaceutical composition of claim 130, wherein the active agent further comprises an additional anti-cancer agent.

132.-167. (canceled)

168. The conjugate of claim 1, wherein the linker is non-cleavable.

169. The conjugate of claim 1, wherein the sortilin binding moiety is or comprises a cyclic peptide, and wherein the payload is or comprises an siRNA.
